(19) United States
(12) Patent Application Publication
(10) Pub. No.: US 2026/0253156 A1
(43) Pub. Date: Aug. 27, 2026

(54) SYSTEM AND METHODS FOR IMPLEMENTING AN EXCHANGE TO EXPEDITE NEGOTIATIONS

(71) Applicant: PharmaCCX, Inc., Boston, MA (US)

(72) Inventors: Peter RICE, Vienna, VA (US); Armen SOLAKHYAN, Mill Valley, CA (US); Cole BOSKEY, Yarmouth Port, MA (US); Alexander KLIPPEL, San Marcos, CA (US)

(21) Appl. No.: 19/107,293

(22) PCT Filed: Aug. 30, 2023
(86) PCT No.: PCT/US2023/031565

§ 371 Date: Feb. 27, 2025

Related U.S. Application Data
(60) Provisional application No. 63/402,866, filed on Aug. 31, 2022.

(51) Int. Cl. G06Q 50/18; G16H 20/10

(52) U.S. Cl. CPC G06Q 50/188; G16H 20/10

(57) ABSTRACT

The present disclosure relates to securing distribution of a pharmaceutical therapy for at least one patient using shared modeling between parties including restricted modes of sharing. Shared pharmaceutical distribution models can include a plurality of distribution constraints with time-varying behavior of at least one parameter that can be simulated and used to evaluate performance metrics. Following simulation a pharmaceutical distribution model can be adjusted by a first party and shared with a second party.

### CROSS REFERENCE TO RELATED APPLICATIONS

This application claims the benefit of U.S. Provisional Application No. 63/402,866, filed Aug. 31, 2022, the contents of which are incorporated herein in its entirety for all purposes.

### FIELD OF THE INVENTION

The present specification relates to systems, methods, and computer program products to share and backtest computer-implemented models having modes of restricted access to expedite negotiations to distribute a pharmaceutical therapy.

### BACKGROUND

Parties, such as suppliers and distributors of pharmaceutical therapies, must establish agreements for the supply and distribution of those pharmaceutical therapies if they are to reach patients in need. Typically, these agreements take months to finalize with each party analyzing and adjusting the agreement in isolation before sending to other participants, resulting in duplicative analysis. The lack of insight that the parties have into their respective counter-party's evaluation of a proposed agreement produces undesirable effects, such as increasing the number of negotiation rounds, lengthening the time until the agreement is finalized, and disputes that may pause or shut down negotiations.

### BRIEF SUMMARY

Certain embodiments may provide, for example, a computer-implemented method to address the above-noted deficiencies. In certain embodiments, for example, the computer-implemented method may include computer-based techniques that enable shared modeling (for example with adjustable modes of restricted access to models) of a draft pharmaceutical distribution agreement. In certain embodiments, for example, the shared modeling may reduce duplicative analysis by stakeholders (for example, suppliers including pharmaceutical companies, distributors including purchasers, brokers, and payers such as insurance companies and government entities, regulatory agents, etc.). In certain embodiments, for example, the shared modeling may reduce the number of issues in dispute in a negotiation and reduce the amount of time it takes to negotiate a pharmaceutical distribution agreement.

A. In certain embodiments, for example, the method may be implemented in a sandbox that allows users of a computing system to generate and simulate test models for distribution of pharmaceutical therapies. In certain embodiments, for example, the users may include one or more users of a supplier computing system. In certain embodiments, for example, the users may include one or more users of a distributor computing system. In certain embodiments, for example, the users may interact with the sandbox through, for example, graphical user interface (GUI) that allows the users to generate a new model or modify an existing model in development, by for example, adding, removing, or adjusting distribution constraints within the model.

B. In certain embodiments, for example, the sandbox is provided by the third-party system and made accessible to the users. In certain embodiments, for example, the method may be implemented in a platform that is hosted on a website. In certain embodiments, for example, access (for example full access or limited access) to the platform may be provided via a URL.

In certain embodiments, for example, the method may facilitate agreement between multiple parties for distributing a pharmaceutical therapy (for example distributing a pharmaceutical therapy to patients). In certain embodiments, for example, the method may enable a first party of the multiple parties and a second party of the multiple parties to identify common areas of agreement (for example win-win provisions) with respect to the variables and/or terms of a proposed distribution agreement.

In certain embodiments, for example, the method may enable a party of the multiple parties (or each of the parties) to perform computer simulations (for example using software implemented using a programming language such as JAVA) with respect to variables and/or terms of a proposed distribution agreement (for example prior to negotiation of or during negotiation of the proposed distribution agreement). In certain embodiments, for example, the computer simulation may track targets (for example goals such as distribution goals for the pharmaceutical therapy) of the party with respect to the proposed distribution agreement over a prospective time horizon.

In certain embodiments, for example, the method may reduce the time to close the proposed distribution agreement by sharing simulation results. In certain embodiments, for example, the method may reduce the time to close the proposed distribution agreement by reducing redundant analysis. In certain embodiments, for example, the method may reduce the time to close the proposed distribution agreement by consolidating deal information on a shared or at least partially shared analytical platform.

In certain embodiments, for example, the method may enable a first party of the multiple parties to share the results of the simulation with a second party of the multiple parties. In certain embodiments, for example, the method may reduce the time to close the proposed distribution agreement by defining one or more simulation scenarios of the proposed distribution agreement as a basis for negotiation of terms of the proposed distribution agreement.

In certain embodiments, for example, the method may enable an intermediary between the multiple parties (for example an arranger of the proposed distribution agreement or a provider of a platform that implements the method) to share the results of the simulation with a party of the multiple parties. In certain embodiments, for example, the method may further comprise a first party of the multiple parties inviting (for example via email containing a URL) and/or onboarding a second party of the multiple parties to access variables and/or terms and or simulation results (for example simulation results for a scenario) associate with the method for the proposed distribution agreement. In certain embodiments, for example, a URL received by the second party may provide restricted access to features of the pharmaceutical distribution model, computer simulation functionality, and/or results of calculations associated with the pharmaceutical distribution model (for example, computational results, computational scenarios, performance metric formulas and/or calculations associated with the pharmaceutical distribution model, etc.) compared to the access enjoyed by the first party. In certain embodiments, for example, the first party may define one or more restricted modes of access to the computational results, computational scenarios, performance metric formulas and/or calculations associated with the pharmaceutical distribution model, etc. In certain embodiments, for example, the first party (or an intermediary between the first party and the second party) may control portions of the system, computer program product, associated computer-implemented method, data, simulation results etc. that are available to the second party, and may define modes that implement such controls. In certain embodiments, for example, the second party's access may be limited (for example read-only access or no access) to portions of the system, computer program product, associated computer-implemented method, data, simulation results etc.

In certain embodiments, for example, the method may enable a party of the multiple parties to wargame the proposed distribution agreement. In certain embodiments, for example, the method may enable a first party of the multiple parties to translate terms of the distribution agreement into related terms for a second party of the multiple parties (for example translating the first party's production budget for the pharmaceutical therapy into a second party's consumption budget and/or revenue or expense stream). In certain embodiments, for example, the computer simulation may be implemented in computer executable software that generates a string for display to a party of the multiple parties. In certain embodiments, for example, the string may be associated with a field that is displayed via a computer-interface to the party. In certain embodiments, for example, the string my include distribution agreement information in a configurable format (for example information displayed on a per-contract basis, consolidated basis, etc.).

In certain embodiments, for example, the method may enable creation of a non-transitory computer readable media containing deal terms for one or more pharmaceutical therapy distribution agreements. In certain embodiments, for example, the method may enable creation of a non-transitory computer readable media containing simulation information (for example simulation results and/or predictions) for one or more pharmaceutical therapy distribution agreements. In certain embodiments, for example, the method may enable creation of a non-transitory computer readable media containing historical tracking information (for example a time series of distribution amounts for the pharmaceutical therapy) for one or more pharmaceutical therapy distribution agreements that has been implemented. In certain embodiments, for example, the method may enable creation of a non-transitory computer readable media containing a comparison of historical tracking information and simulation information for one or more pharmaceutical therapy distribution agreements that has been implemented.

In certain embodiments, for example, the method may enable post-negotiation analysis of a pharmaceutical distribution agreement. In certain embodiments, for example, the method may enable back-testing a proposed pharmaceutical distribution agreement.

C. In certain embodiments, for example, the pharmaceutical therapy may comprise a diagnosis. In certain embodiments, for example, the pharmaceutical therapy may comprise a code (for example a diagnosis code, a regimen code, or a billing code). In certain embodiments, for example, the pharmaceutical therapy may be associated with a therapy type (for example monotherapy or combination therapy). In certain embodiments, for example, the pharmaceutical therapy may be associated with a market. In certain embodiments, for example, the pharmaceutical therapy may comprise a treatment (for example administration of a drug) to treat a condition (for example a disease). In certain embodiments, for example, the pharmaceutical therapy may comprise a treatment to prevent a condition. In certain embodiments, for example, the pharmaceutical therapy may comprise a treatment regimen.

In certain embodiments, for example, the pharmaceutical therapy may comprise a single pharmaceutical drug. In certain embodiments, for example, the pharmaceutical therapy may comprise multiple pharmaceutical drugs. In certain embodiments, for example, the pharmaceutical therapy may comprise a single dose of a pharmaceutical drug. In certain embodiments, for example, the pharmaceutical therapy may comprise multiple does of one or more pharmaceutical drugs. In certain embodiments, for example, the pharmaceutical therapy may comprise therapy (for example, radiation therapy, physical therapy, etc.). In certain embodiments, for example, the pharmaceutical therapy may comprise a combination of therapy and one or more doses of one or more pharmaceutical drugs. In certain embodiments, for example, the pharmaceutical therapy may comprise a combination of two or more of the foregoing.

In certain embodiments, for example, all or part of the treatment is approved by a regulatory body. In certain embodiments, for example, all or part of the treatment is in the process of seeking regulatory approval. In certain embodiments, for example, the pharmaceutical therapy may be a pharmaceutical therapy for a patient in need of the pharmaceutical therapy (for example a patient suffering from a condition that may be treated with the pharmaceutical therapy). In certain embodiments, for example, a patient may be a person having or diagnosed with a condition that is treatable with the treatment. In certain embodiments, a patient is an eligible patient that meets, for example, one or more additional criteria, such as being a person in a particular region, not having another condition that might be worsened by the treatment, not taking a particular pharmaceutical drug that interacts with the treatment, being at least a certain age that the treatment is approved for, having insurance or a particular insurance, or a combination thereof.

In certain embodiments, for example, the treatment regimen may comprise a dosing schedule for a drug. In certain embodiments, for example, the dosing schedule may comprise a start cycle, a cycle length, an end cycle, and a dosing for all days in each of the foregoing cycles. In certain embodiments, for example, the dosing may comprise a number of administrations of a product (for example a pharmaceutical drug) per day and a dosage amount per administration. In certain embodiments, for example, the dosing schedule may comprise a dosing for all days In certain embodiments, for example, the pharmaceutical therapy may comprise a new drug therapy. In certain embodiments, for example, the pharmaceutical therapy has an associated product pack. In certain embodiments, for example, the product pack may be based on an average dosage of a pharmaceutical compound to be administered to a patient within a pre-determined period of time.

Certain embodiments of the method may comprise, for example, configuring a computer-executable pharmaceutical distribution model (based on, for example, input from a user such as a supplier or purchaser of the pharmaceutical therapy) that comprises a plurality of distribution constraints.

A. In certain embodiments, for example, the configuring may comprise receiving input data from a user.

B. In certain embodiments, for example, the computer-executable pharmaceutical distribution model may be accessible to the user via a website interface. In certain embodiments, for example, the computer-executable pharmaceutical distribution model may be accessible to the user via a downloadable app. In certain embodiments, for example, the computer-executable pharmaceutical distribution model may be configured to execute remotely from the user. In certain embodiments, for example, the computer-executable pharmaceutical distribution model may be configured to execute on a computer device under the control of the user. In certain embodiments, for example, the computer-executable pharmaceutical distribution model may be configured in a collaborative mode. In certain embodiments, for example, the computer-executable pharmaceutical distribution model may be configured for use by two or more uses at the same time, for example by two different users operating separate computing devices (for example, computer devices that are remote (different office, city, state, country, etc.). In certain embodiments, for example, the computer-executable pharmaceutical distribution model may be installed on a cloud (or other distributed computing environment) that provides access to the computer-executable pharmaceutical distribution model by multiple users.

In certain embodiments, for example, the computer-executable pharmaceutical distribution model may comprise a distribution model for distribution of an alternative pharmaceutical therapy to the pharmaceutical therapy (for example an alternative pharmaceutical therapy that specifies a different pharmaceutical compound, treatment regimen, etc.).

C. In certain embodiments, for example, the plurality of distribution constraints may define a supply amount as a function, at least in part, of an effectiveness of the pharmaceutical therapy (for example, the percentage of patients that have their condition successfully cured, corrected, or maintained as a result of being treated with the therapy; the percentage of patients that do not require a change in a pharmaceutical drug and/or dosage of the therapy to treat adequately treat their condition; a metric that indicates a length of time that a patient is required to take the therapy to treat their condition; a metric that indicates a number of doses/packs/or units of a therapy needed per patient to treat a condition, etc.). For example, a supply amount required per year may be defined as equal to the estimated number of patients per year multiplied by a number of doses/packs/units of the therapy required per patient multiplied by the effectiveness of the therapy. A more complex function may also take into account additional supply that would be used to treat a set of patients, for which the therapy is ultimately not effective, until a determination is made that the therapy is not effective for that set of patients (for example, additional supply needed as a patient may need to be treated with one, two, three, or four doses/packs/units of the therapy before a determination that the therapy is not effective for that patient can be accurately made).

In certain embodiments, for example, the plurality of distribution constraints may define a supply amount as a function, at least in part, of a size of a patient population (for example a time-dependent size of a patient population). In certain embodiments, for example, the plurality of distribution constraints may define a supply amount as a function, at least in part, of a number of treatments per patient (for example a number of treatments per patient over an expected course of the pharmaceutical therapy, a number of treatments per patient during a given time, a number of treatment cycles per year, etc.). In certain embodiments, for example, the plurality of distribution constraints may define a supply amount as a function, at least in part, of patient adherence to the pharmaceutical therapy (for example, the percentage of patients in a patient population that complete a prescribed number of treatment cycles of the pharmaceutical therapy; the percentage of patients in a patient population that adhere to a treatment schedule for the pharmaceutical therapy; the percentage of patients in a patient population that adhere to a treatment schedule within a threshold of time such as within 30 minutes, one hour, two hours, 12 hours, or 24 hours of a scheduled treatment time; the percentage of patients in a patient population that the percentage of patients in a patient population that adhere to a treatment schedule and complete a prescribed number of treatment cycles; the he percentage of patients in a patient population that the percentage of patients in a patient population that adhere to a treatment schedule within a threshold of time and complete a prescribed number of treatment cycles; etc.).

In certain embodiments, for example, the plurality of distribution constraints may comprise constraints on one or more funding amounts for the distribution of the pharmaceutical therapy. In certain embodiments, for example, the constraints on one or more funding amounts may comprise an upfront payment (for an up front payment on a per-patient basis). In certain embodiments, for example, constraints on one or more funding amounts may comprise a further payment that is contingent upon a patient response to the pharmaceutical therapy (for example a positive per-patient response or a measure of an aggregate response to the pharmaceutical therapy by a plurality of patients). In certain embodiments, for example, the further payment may be larger than the upfront payment. In certain embodiments, for example, constraints on one or more funding amounts may comprise a rebate (for example a rebate paid after purchase of a pharmaceutical therapy) that is contingent upon a patient response to the pharmaceutical therapy (for example a negative per-patient response or a measure of an aggregate response to the pharmaceutical therapy by a plurality of patients). In certain embodiments, for example, the rebate may be smaller than the upfront payment. In certain embodiments, for example, the constraints on one or more funding amounts may comprise a discount (for example an upfront discount that reduces a funding amount paid) to a funding amount of the one or more funding amounts as a function of a total number of patients that receive the pharmaceutical therapy. In certain embodiments, for example, the discount may be applied on a per-patient basis. In certain embodiments, for example, availability of the discount may require a specified level of patient adherence to the pharmaceutical therapy. In certain embodiments, for example, the discount may be tiered for different patients in a tier (for example tiers defined by geographic location of a patient, patient age, patient comorbidities, etc.). In certain embodiments, for example, the constraints on one or more funding amounts comprise a discount to a funding amount of the one or more funding amounts as a function of a previous funding provided during a prior specified time period (for example, a discount applied to future fundings during a calendar year when previous fundings exceeds a predetermined amount within the calendar year).

In certain embodiments, for example, the constraints on one or more funding amounts may comprise a cap on funding during a specified time period (for example a cap on funding expenditures during a calendar year on a per-patient basis). In certain embodiments, for example, the cap may be a cap on total funding during a specific time period. In certain embodiments, for example, the cap may be a limit on payments to payments incurred during a specified portion of a time period (for example, a limit on payments for receipt of the pharmaceutical therapy to those payments incurred during the first 10 months of a calendar year). In certain embodiments, for example, the cap may be applied on a per patient basis. In certain embodiments, for example, the cap may be an aggregate cap for a group of patients (for example a population of patients, such as a population of patients in a geographic region). In certain embodiments, for example, the constraints on one or more funding amounts may further comprise a rebate that is triggered once the one or more funding amounts reaches the aggregate cap. In certain embodiments, for example, the rebate may be effective for a specified incremental amount above the aggregate cap (for example, if the aggregate cap is $100 million and the incremental funding amount is $30 million, then a recipient of the pharmaceutical therapy in the about of $131 million is responsible for funding the first $100 million and the last $1 million for a total of $101 million).

In certain embodiments, for example, the constraints on one or more funding amounts comprise a periodic funding amount for a specified number of periods (for example a specified funding amount for each year of five years). In certain embodiments, for example, the periodic funding amount for the specified number of periods may fund treatment of an unlimited number of patients with the pharmaceutical therapy during the specified number of periods. In certain embodiments, for example, the constraints on one or more funding amounts may comprise a specified number of free treatments (for example an initial number of free treatments or an additional number of free treatments following funding of an initial number of treatments) with the pharmaceutical therapy. In certain embodiments, for example, the number of free treatments may be determined from a number of funded treatments. In certain embodiments, for example, the constraints on one or more funding amounts may comprise a deferral of funding for a set of treatments with the pharmaceutical therapy until the pharmaceutical therapy receives regulatory approval. In certain embodiments, for example, the funding may be at a discounted rate relative to a price of the pharmaceutical therapy that is established based on approval of the pharmaceutical therapy.

D. In certain embodiments, for example, the plurality of distribution constraints may comprise equalities. In certain embodiments, for example, the plurality of distribution constraints may comprise inequalities.

In certain embodiments, for example, the computer executable pharmaceutical distribution model may be defined, at least in part, by a constraint that specifies a minimum supply of a treatment per year. In certain embodiments, for example, the computer executable pharmaceutical distribution model may be defined, at least in part, by a constraint that specifies a time horizon (for example, a period of months or years) over which a pharmaceutical distribution agreement is to be analyzed. In certain embodiments, for example, the computer executable pharmaceutical distribution model may be defined, at least in part, by a constraint that specifies anticipated number of patients per year. In certain embodiments, for example, the computer executable pharmaceutical distribution model may be defined, at least in part, by a constraint that specifies that a minimum number of patients must be less than or equal to the anticipated number of patients.

In certain embodiments, for example, the plurality of distribution constraints may comprise a bounding value (for example a maximum value or a minimum value) for a supply amount of the pharmaceutical therapy. In certain embodiments, for example, the bounding value for the supply amount may be computed from an incremental change (for example a minimum increase, a minimum reduction, a maximum increase, or a maximum reduction) to a supply amount of the pharmaceutical therapy relative to an initially specified supply mount of the pharmaceutical therapy.

In certain embodiments, for example, the bounding value may be a function, at least in part, of an effectiveness of the pharmaceutical therapy (for example a known effectiveness, an average effectiveness, a predicted effectiveness, a simulated effectiveness, or an optimized effectiveness). In certain embodiments, for example, the bounding value may be computed from an incremental change (for example a minimum increase, a minimum reduction, a maximum increase, or a maximum reduction) to the supply amount that is triggered by a threshold level of deviation of the effectiveness from an initial expectation of the effectiveness of the pharmaceutical therapy.

In certain embodiments, for example, the bounding value may be a function, at least in part, of a realized size of a patient population for the pharmaceutical therapy. In certain embodiments, for example, the bounding value may be computed from an incremental change (for example a minimum increase, a minimum reduction, a maximum increase, or a maximum reduction) to the supply amount that is triggered by a threshold level of deviation of the realized size from an initial expectation of the size of the patient population.

In certain embodiments, for example, the bounding value may be a function, at least in part, of a number of treatments per patient. In certain embodiments, for example, the bounding value may be computed from an incremental change (for example a minimum increase, a minimum reduction, a maximum increase, or a maximum reduction) to the supply amount that is triggered by a threshold level of deviation of the realized number of treatments per patient from an initial expectation of the number of treatments per patient.

In certain embodiments, for example, the bounding value may be a function, at least in part, of a number of discontinuation recommendations for the pharmaceutical therapy.

In certain embodiments, for example, the bounding value may be computed from an incremental change (for example a minimum increase, a minimum reduction, a maximum increase, or a maximum reduction) to the supply amount that is triggered by a threshold level of deviation of the number of discontinuation recommendations from an initial expectation of the effectiveness of the number of discontinuation recommendations.

In certain embodiments, for example, the bounding value may be a function, at least in part, of an available supply of the pharmaceutical therapy. In certain embodiments, for example, the bounding value may be computed from an incremental change (for example a minimum increase, a minimum reduction, a maximum increase, or a maximum reduction) to the supply amount that is triggered by a threshold level of deviation of the available supply from an initial expectation of the available supply.

In certain embodiments, for example, the bounding value may be a function, at least in part, of a regulatory status of the pharmaceutical therapy. In certain embodiments, for example, the bounding value may be computed from an incremental change (for example a minimum increase, a minimum reduction, a maximum increase, or a maximum reduction) to the regulatory status of the pharmaceutical therapy that is triggered by a threshold level of deviation of the regulatory status from an initial expectation of the regulatory status.

In certain embodiments, for example, the bounding value may be a function, at least in part, of a geographical demand (for example demand by a region, country, state, county, etc.) for the pharmaceutical therapy. In certain embodiments, for example, the bounding value may be computed from an incremental change (for example a minimum increase, a minimum reduction, a maximum increase, or a maximum reduction) to the geographical demand of the pharmaceutical therapy that is triggered by a threshold level of deviation of the geographical demand from an initial expectation of the geographical demand.

In certain embodiments, for example, the bounding value may be a function, at least in part, of a legal constraint (for example an export ban, import ban, tariff, etc.) on the pharmaceutical therapy. In certain embodiments, for example, the bounding value may be computed from an incremental change (for example a minimum increase, a minimum reduction, a maximum increase, or a maximum reduction) to the legal constraint that is triggered by a threshold level of deviation of the legal constraint from an initial expectation of the legal constraint or lack thereof.

In certain embodiments, for example, the bounding value may be a function, at least in part, of a governmental factor (for example a government subsidy). In certain embodiments, for example, the bounding value may be computed based on a change to a governmental factor (for example the allowance or withdrawal of a government subsidy for the pharmaceutical therapy, or a government reimbursement rate for the pharmaceutical therapy).

In certain embodiments, for example, the bounding value may be a function, at least in part, of a reimbursement rate for the pharmaceutical therapy. In certain embodiments, for example, the bounding value may be computed from a change (for example a minimum increase, a minimum reduction, a maximum increase, or a maximum reduction) to the reimbursement rate of the pharmaceutical therapy.

E. In certain embodiments, for example, a constraint of the plural distribution constraints may comprise a discount that depends on the total number of the one or more patients to which the pharmaceutical therapy is distributed.

F. In certain embodiments, for example, the configuring may comprise setting a value for a parameter of a plurality of parameters (or values for the plurality of parameters) associated with the distribution constraints. In certain embodiments, for example, the plurality of parameters may comprise coefficients of variables in the distribution constraints. In certain embodiments, for example, the plurality of parameters may comprise binary variables that multiplies at least one distribution constraint of the plurality of distribution constraints. In certain embodiments, for example, a parameter of the plurality of parameters may correspond to a variable that is associated with the plurality of distribution constraints. In certain embodiments, for example, the variable may be a continuous variable. In certain embodiments, for example, the variable may be a discrete variable. In certain embodiments, for example, the plurality of distribution constraints may comprise a distribution constraint that defines a bound on the variable (for example, an upper bound and/or a lower bound).

In certain embodiments, for example, the value may be generated using a computer-implemented statistical model. In certain embodiments, for example, the value may be one of a set of values corresponding to the parameter at different instances in time. In certain embodiments, for example, the value of one of a set of values corresponding to multiple scenarios for the parameter. In certain embodiments, for example, the configuring may comprise setting a value corresponding to a parameter of a plurality of parameters that are associated with the plurality of distribution constraints. In certain embodiments, for example, the plurality of parameters may comprise coefficients of variables in the plurality of distribution constraints. In certain embodiments, for example, the plurality of parameters may comprise variables in the plurality of distribution constraints. In certain embodiments, for example, the plurality of parameters comprise a binary variable that multiplies a distribution constraint of the plurality of distribution constraints.

In certain embodiments, for example, a parameter of the plurality of parameters may include a region where the pharmaceutical therapy is to be distributed. In certain embodiments, for example, a parameter of the plurality of parameters may include a dosage of a pharmaceutical drug in the treatment. In certain embodiments, for example, a parameter of the plurality of parameters may include a number of doses of a pharmaceutical drug in the treatment required or anticipated per patient. In certain embodiments, for example, a parameter of the plurality of parameters may include a number of persons in a region diagnosed with a condition that is treatable using the pharmaceutical therapy. In certain embodiments, for example, a parameter of the plurality of parameters may include a distributor's cost per treatment.

G. In certain embodiments, for example, the distribution constraints may comprise a plurality of archetypal constraints. In certain embodiments, for example, a first archetypal constraint of the plurality of archetypal constraints may be mutually exclusive to a second archetypal constraint of the plurality of archetypal constraints.

H. In certain embodiments, for example, the configuring may comprise generating source code for the computer-executable pharmaceutical distribution model. In certain embodiments, for example, the computer-executable pharmaceutical distribution model may be implemented on a server. In certain embodiments, for example, the computer-executable model may be configured to be executed by an analytical engine. In certain embodiments, for example, the configuring may be guided at least in part by a distributor of the pharmaceutical therapy operating a computing device. In certain embodiments, for example, the configuring may be guided at least in part by a producer of the pharmaceutical therapy operating a computing device. In certain embodiments, for example, the configuring may be guided at least in part by an intermediary operating on behalf of a group of patients operating a computing device. In certain embodiments, for example, the configuring may be guided at least in part by a patient operating a computing device.

In certain embodiments, for example, the configuring may be guided at least in part by an intermediary between a first distributor of the pharmaceutical therapy and a second distributor of the pharmaceutical therapy. In certain embodiments, for example, the first distributor of the pharmaceutical therapy may be a supplier (for example a manufacturer) of at least one component (for example a pharmaceutical drug or an active ingredient in a pharmaceutical drug) of the pharmaceutical therapy.

I. In certain embodiments, for example, the method may further comprise generating a plurality of computer-executable pharmaceutical distribution models. In certain embodiments, for example, the method may further comprise computing a ranking of a first model of the plurality of computer-executable pharmaceutical distribution models relative to a second model of the plurality of computer-executable pharmaceutical distribution models. In certain embodiments, for example, the ranking may be based on relevance. In certain embodiments, for example, relevance may be based at least in part on a specified drug dosage. In certain embodiments, for example, relevance may be based at least in part on a duration of the pharmaceutical therapy. In certain embodiments, for example, relevance may be based at least in part on a specified region, country, state, county, or other geographic subdivision. In certain embodiments, for example, the ranking may be displayed on a user interface.

J. In certain embodiments, information used to generate the pharmaceutical distribution model may be obtained, at least in part, from (or input from) one or more external sources of information (for example, an external source of information such as a database of a medical insurance provider, an academic database storing medical information, etc.), one or more internal sources (for example an internal source of information such as information relied on for previously generated models, medical data received from one or more distributors of treatments, medical data received from one or more suppliers of treatments, etc.), or a combination of external and internal sources.

Certain embodiments for the method may comprise, for example, calculating a time-varying behavior of at least one parameter (for example a parameter of a plurality of parameters) of the computer-executable pharmaceutical distribution model to obtain computational results.

A. In certain embodiments, for example, calculating the time-varying behavior may comprise performing calculations to enforce a distribution constraint of the plurality of distribution constraints. In certain embodiments, for example, the calculations to enforce the distribution constraint may comprise determining a value for a variable that allows the distribution constraint to be enforced. In certain embodiments, for example, calculating the time-varying behavior may comprise performing calculations to determine a time series of values for a variable present in a distribution constraint of the plurality of distribution constraints. In certain embodiments, for example, the calculating the time-varying behavior may comprise a dynamic simulation. In certain embodiments, for example, the calculating the time-varying behavior may comprise calculating the time-varying behavior of the at least one parameter for a scenario. In certain embodiments, for example, the calculating the time-varying behavior may comprise calculating the time-varying behavior of the at least one parameter for a plurality of scenarios. In certain embodiments, for example, the calculating the time-varying behavior may comprise a Monte Carlo simulation.

In certain embodiments, for example, the calculating the time-varying behavior may be performed during an optimization process (for example optimization of one or more parameters of the at least one parameter with respect to a performance metric, such as maximizing or minimizing the performance metric through the selection of the one or more parameters). In certain embodiments, for example, the optimization process may comprise at least partially solving a linear program. In certain embodiments, for example, the optimization process may comprise at least partially solving a nonlinear program. In certain embodiments, for example, the optimization process may comprise at least partially solving an integer program. In certain embodiments, for example, the optimization process may comprise at least partially solving an integer-linear program. In certain embodiments, for example, the optimization process may comprise at least partially solving a mixed integer-nonlinear program. In certain embodiments, for example, the optimization process may comprise simulated annealing. In certain embodiments, for example, the optimization process may at least partially optimizes the at least one parameter. In certain embodiments, for example, the parameter may be a variable associated with the plurality of distribution constraints. In certain embodiments, for example, the parameter may be a coefficient of a variable that may be associated with the plurality of distribution constraints. In certain embodiments, for example, the parameter may be a binary parameter that multiples another parameter that is associated with the plurality of distribution constraints.

B. In certain embodiments, for example, the time-varying behavior may comprise a change (for example an instantaneous change or a cumulative change) in a distribution quantity of the pharmaceutical therapy. In certain embodiments, for example, the time-varying behavior may comprise a change in a patent adherence to the pharmaceutical therapy. In certain embodiments, for example, the time-varying behavior may comprise a change in a residual of a distribution constraint of the plurality of distribution constraints. In certain embodiments, for example, the time-varying behavior may comprise a change in a supply of the pharmaceutical therapy. In certain embodiments, for example, the time-varying behavior may comprise a change in a number of discontinuation recommendations for the pharmaceutical therapy. In certain embodiments, for example, the time-varying behavior may comprise a change in an available supply for the pharmaceutical therapy. In certain embodiments, for example, the time-varying behavior may comprise a change in a production rate for the pharmaceutical therapy. In certain embodiments, for example, the time-varying behavior may comprise a change in a regulatory status (for example regulatory approval or regulatory rejection) for the pharmaceutical therapy. In certain embodiments, for example, the time-varying behavior may comprise a change in a legal status for the pharmaceutical therapy. In certain embodiments, for example, the time-varying behavior may comprise a change in a geographic factor (for example a geographic supply factor or a geographic demand factor) for the pharmaceutical therapy. In certain embodiments, for example, the time-varying behavior may comprise a change in a governmental factor (for example a government subsidy) for the pharmaceutical therapy. In certain embodiments, for example, the time-varying behavior may comprise a change in a reimbursement rate (for example an insurance reimbursement rate for the pharmaceutical therapy.

C. In certain embodiments, for example, the computational results may comprise a residual of a distribution constraint of the plurality of distribution constraints. In certain embodiments, for example, the computational results may comprise a selection of constraints from the plurality of distribution constraints. In certain embodiments, for example, the computational results may comprise residual values for a constraint of the plurality of distribution constraints. In certain embodiments, for example, the computational results may comprise a time series of values for a variable associated with the plurality of distribution constraints.

D. In certain embodiments, for example, the computer-executable pharmaceutical distribution model may comprise fixed coefficients. In certain embodiments, for example, the fixed coefficients may be obtained from a third-party data source. In certain embodiments, for example, the method may further comprise computing some or all of the fixed coefficients.

Certain embodiments of the method may comprise, for example, quantifying a value of a first performance metric for the computational results.

A. In certain embodiments, for example, the first performance metric may comprise a number of patients treated using the pharmaceutical therapy during one or more specified time periods. In certain embodiments, for example, the first performance metric may comprise a size of a patient population that is eligible to receive the pharmaceutical therapy during one or more specified time periods (or points in time). In certain embodiments, for example, the first performance metric may comprise a metric of patient adherence to the pharmaceutical therapy during one or more specified time periods (or points in time). In certain embodiments, for example, the first performance metric may comprise a measure of funding associated with the distribution of the pharmaceutical therapy. In certain embodiments, for example, the first performance metric may comprise a measure of return (for example, total return, margin, yield) associated with the distribution of the pharmaceutical therapy. In certain embodiments, for example, the first performance metric may comprise a measure of financial impact on patients associated with use of the pharmaceutical therapy. In certain embodiments, for example, the first performance metric may comprise a measure of the number of discontinuation recommendations for the pharmaceutical therapy. In certain embodiments, for example, the first performance metric may comprise a price (for example a price-per-patient, a price-per-pack, or a price ratio of prices in different countries, states, counties or other regions as the case may be) for the pharmaceutical therapy (for example a future price, an average price over a specified time period, or a price in a specified geography). In certain embodiments, for example, the first performance metric may comprise a measure of geographic breadth of distribution of the pharmaceutical therapy.

B. In certain embodiments, for example, the quantifying may comprise calculating an average (for example a simple average or a weighted average) of measurements of the first performance metric for a plurality of scenarios. In certain embodiments, for example, the quantifying may comprise calculating a statistical measure the first performance metric (for example, a statistical measure the first performance metric based on a plurality of scenarios). In certain embodiments, for example, the quantifying may comprise calculating a weighted average of measurements of the first performance metric for a plurality of scenarios. In certain embodiments, for example, the measurements of the first performance metric for the plurality of scenarios may be weighted by eliminating a maximum measurement of the first performance metric and/or a minimum measurement of the first performance metric.

Certain embodiments of the method may comprise, for example, adjusting the plurality of distribution constraints based at least on the value of the first performance metric to form a computer-executable adjusted pharmaceutical distribution model that comprises an adjusted plurality of distribution constraints. In certain embodiments, for example, the adjusting may be further based at least on the computational results. In certain embodiments, for example, the adjusting may comprise replacing a parameter (for example a coefficient or a variable) in the plurality of distribution constraints with a fixed value. In certain embodiments, for example, the fixed value may be determined from an optimization (for example optimization with respect to a performance metric) with respect to the parameter. In certain embodiments, for example, the adjusting may comprise removing one or more of the distribution constraints from the plurality of distribution constraints. In certain embodiments, for example, the adjusting may comprise adding a new distribution constraint to the plurality of distribution constraints.

C. In certain embodiments, for example, adjusting may comprise recommending one or more adjustments to the pharmaceutical distribution model to a user wherein the user takes one or more actions through an interactive computer interface to approve and/or modify the recommended one or more adjustments to select one or more features of the adjusted pharmaceutical distribution model. In certain embodiments, for example, the method comprises user feedback including approval of the recommended one or more adjustments, disapproval of the recommended one or more adjustments, or edits to the recommended one or more adjustments.

D. In certain embodiments, for example, the adjusted pharmaceutical model may define or implement a restricted mode of the pharmaceutical distribution model. In certain embodiments, for example, the adjusted pharmaceutical distribution model may have lower dimensionality (for example fewer variables and/or fewer constraints) than the pharmaceutical distribution model.

Certain embodiments of the method may comprise, for example, further calculating a time-varying behavior of at least one further parameter of the computer-executable adjusted pharmaceutical distribution model to obtain adjusted computational results. In certain embodiments, for example, the at least one further parameter may corresponds to the at least one parameter. In certain embodiments, for example, the at least one further parameter does not completely correspond to the at least one parameter.

Certain embodiments of the method may comprise, for example, further quantifying a value of a second performance metric for the adjusted simulation results. In certain embodiments, for example, the second performance metric may correspond to the first performance metric (for example the second performance metric is the same performance metric as the first performance metric). In certain embodiments, for example, the second performance metric may not correspond to the first performance metric.

Certain embodiments of the method may comprise, for example, making the computer-executable adjusted pharmaceutical distribution model accessible to a remote (for example third party) computing device. In certain embodiments, for example, the method may further comprise making the adjusted computational results available to the third-party computing device. In certain embodiments, for example, the method may further comprise making the value of the second performance metric available to the third party computing device.

Certain embodiments may provide, for example, a computer-implemented method for distributing a pharmaceutical therapy to at least one patient, comprising: [i] configuring a computer-executable pharmaceutical distribution model that comprises a plurality of distribution constraints; [ii] calculating a time-varying behavior of at least one parameter of the computer-executable pharmaceutical distribution model to obtain computational results; [iii] quantifying a value of a first performance metric for the computational results; [iv] adjusting the plurality of distribution constraints based at least on the computational results and on the value of the first performance metric to form a computer-executable adjusted pharmaceutical distribution model that comprises an adjusted plurality of distribution constraints; [v] further calculating a time-varying behavior of at least one further parameter of the computer-executable adjusted pharmaceutical distribution model to obtain adjusted computational results; [vi] further quantifying a value of a second performance metric for the adjusted simulation results; and [vii] making the computer-executable adjusted pharmaceutical distribution model accessible to a remote (for example third party) computing device.

Certain embodiments may provide, for example, a computer-implemented method for backtesting a pharmaceutical distribution model. In certain embodiments, for example, the method may comprise configuring a computer-executable pharmaceutical distribution model that comprises a plurality of distribution constraints. In certain embodiments, for example, the method may comprise assigning at least one defined scenario for a parameter (or a plurality of parameters) of the computer-executable pharmaceutical distribution model. In certain embodiments, for example, the method may comprise simulating the computer-executable pharmaceutical distribution model based at least in part on the defined scenario to obtain computational results. In certain embodiments, for example, the method may comprise quantifying a value of a performance metric for the computational results. In certain embodiments, for example, the method may comprise defining a data set for the parameter. In certain embodiments, for example, the method may comprise further calculating the time-varying behavior of the at least one parameter based on the historical data set to obtain backtesting computational results. In certain embodiments, for example, the method may comprise further quantifying a value of the performance metric for the backtesting computational results. In certain embodiments, for example, the method may comprise adjusting a distribution constraint of the plurality of distribution constraints based on a difference between the computational results and the backtesting computational results or making the backtesting computational results accessible to a remote (for example third party) computing device.

A. In certain embodiments, for example, the at least one parameter may comprise a time-varying parameter. In certain embodiments, for example, the at least one parameter may comprise a static parameter.

B. In certain embodiments, for example, the data set may be a historical data set. In certain embodiments, for example, the historical data set may be part of historical data results for performance of a previously-implemented pharmaceutical distribution model. In certain embodiments, for example, the previously-implemented pharmaceutical distribution model may comprise a previously-implemented distribution constraint that shares a common archetype with a distribution constraint of the plurality of distribution constraints. In certain embodiments, for example, the previously-implemented pharmaceutical distribution model may comprise selected at least in part based on similarity (for example overlap of archetypes) between one or more previously-implemented distribution constraints and one or more distribution constraints of the plurality of distribution constraints. In certain embodiments, for example, the previously-implemented pharmaceutical distribution model may be selected at least in part based on similarity (for example overlap of archetypes) between a previously-implemented performance metric applied to the previously-implemented pharmaceutical distribution model and the performance metric.

C. In certain embodiments, for example, the data set may be a synthetic data set. In certain embodiments, for example, the data set may be part of simulated computational results for performance of a synthetic pharmaceutical distribution model. In certain embodiments, for example, the synthetic pharmaceutical distribution model may comprise a synthetic distribution constraint that shares a common archetype with a distribution constraint of the plurality of distribution constraints. In certain embodiments, for example, the synthetic pharmaceutical distribution model may be selected at least in part based on similarity (for example overlap of archetypes) between one or more synthetic distribution constraints and one or more distribution constraints of the plurality of distribution constraints. In certain embodiments, for example, the synthetic pharmaceutical distribution model may be selected at least in part based on similarity (for example overlap of archetypes) between a synthetic performance metric applied to the synthetic pharmaceutical distribution model and the performance metric. In certain embodiments, for example, the adjusting may comprise adding a distribution constraint to the plurality of distribution constraints. In certain embodiments, for example, the adjusting may comprise removing a distribution constraint from the plurality of distribution constraints. In certain embodiments, for example, the adjusting may comprise replacing a distribution constraint in the plurality of distribution constraints.

Certain embodiments may provide, for example, a computer-implemented method for backtesting a pharmaceutical therapy to at least one patient, comprising: [i] configuring a computer-executable pharmaceutical distribution model that comprises a plurality of distribution constraints; [ii] assigning at least one defined scenario for a parameter (or a plurality of parameters) of the computer-executable pharmaceutical distribution model; [iii] simulating the computer-executable pharmaceutical distribution model based at least in part on the defined scenario to obtain computational results; [iv] quantifying a value of a performance metric for the computational results; [v] defining a data set for the parameter; [vi] further calculating the time-varying behavior of the at least one parameter based on the historical data set to obtain backtesting computational results; [vii] further quantifying a value of the performance metric for the backtesting computational results; and [viii] adjusting a distribution constraint of the plurality of distribution constraints based on a difference between the computational results and the backtesting computational results or making the backtesting computational results accessible to a remote (for example third party) computing device.

Certain embodiments may provide, for example, a system or a computer program product implementing one or more of the foregoing methods.

Certain embodiments may provide, for example, a product for distributing a pharmaceutical therapy to at least one patient, the product comprising a non-transitory computer-readable storage medium having computer-readable program code executable by a computing device to perform computer modeling and communication operations, the computer modeling and communication operations comprising: [i] configuring a computer-executable pharmaceutical distribution model that comprises a plurality of distribution constraints; [ii] calculating a time-varying behavior of at least one parameter of the computer-executable pharmaceutical distribution model to obtain computational results; [iii] quantifying a value of a first performance metric for the computational results; [iv] adjusting the plurality of distribution constraints based at least on the computational results and on the value of the first performance metric to form a computer-executable adjusted pharmaceutical distribution model that comprises an adjusted plurality of distribution constraints; [v] further calculating a time-varying behavior of at least one further parameter of the computer-executable adjusted pharmaceutical distribution model to obtain adjusted computational results; [vi] further quantifying a value of a second performance metric for the adjusted simulation results; and [vii] making the computer-executable adjusted pharmaceutical distribution model accessible to a remote (for example, third party) computing device.

Certain embodiments may provide, for example, a system for distributing a pharmaceutical therapy to at least one patient, the system comprising an analytic engine and one or more computing systems configured to perform operations comprising: [i] configuring a computer-executable pharmaceutical distribution model that comprises a plurality of distribution constraints; [ii] calculating a time-varying behavior of at least one parameter of the computer-executable pharmaceutical distribution model to obtain computational results; [iii] quantifying a value of a first performance metric for the computational results; [iv] adjusting the plurality of distribution constraints based at least on the computational results and on the value of the first performance metric to form a computer-executable adjusted pharmaceutical distribution model that comprises an adjusted plurality of distribution constraints; [v] further calculating a time-varying behavior of at least one further parameter of the computer-executable adjusted pharmaceutical distribution model to obtain adjusted computational results; [vi] further quantifying a value of a second performance metric for the adjusted simulation results; and [vii] making the computer-executable adjusted pharmaceutical distribution model accessible to a remote (for example, third party) computing device.

In certain embodiments, for example, the system may comprise (or communicate with) one or more computing systems (for example one or more distributed computing systems). In certain embodiments, for example, the system may comprise a supplier computing system under the control of a supplier of the pharmaceutical therapy. In certain embodiments, for example, the system may comprise a distributor computing system under the control of a distributor computing system of the pharmaceutical therapy. In certain embodiments, for example, the system may comprise a third-party computing system under the control of a third party. In certain embodiments, for example, the system may comprise the third-party computing system. In certain embodiments, for example, the third-party computing system may be configured to communicate with the supplier computing system, the distributor computing system, or both the supplier computing system and the distributor computing system.

In certain embodiments, for example, the system may comprise or use multiple supplier computing systems. In certain embodiments, for example, the system may communicate with different computing systems of suppliers of different pharmaceutical therapies. In certain embodiments, for example, the system may comprise or use multiple distributor computing systems. For example, the third-party computing system may communicate with different computing systems of suppliers of different pharmaceutical therapies.

In certain embodiments, for example, the system may obtain treatment information from external sources. In certain embodiments, for example, the system, generating a model for a particular treatment comprises the third-party computing system communicating with one or more databases containing information for the particular treatment. In certain embodiments, for example, this information may include information about the number of persons suffering from a condition that is treatable with the treatment, information about the number of treatments required per patient, information about the amount of time that patients require from a start of the treatment or a first treatment until an end of the treatment or a last treatment, information indicating trends in the length of treatment or the number of persons suffering from a condition that is treatable with the treatment, information about other suppliers of the treatment, information about other treatments that are able to treat a condition that is also treatable by the treatment, etc.

Certain embodiments may provide, for example, a product for backtesting a pharmaceutical distribution model, the product comprising a non-transitory computer-readable storage medium having computer-readable program code executable by a computing device to perform computer simulation and communication operations, the computer modeling and communication operations comprising: [i] configuring a computer-executable pharmaceutical distribution model that comprises a plurality of distribution constraints; [ii] assigning at least one defined scenario for a parameter (or a plurality of parameters) of the computer-executable pharmaceutical distribution model; [iii] simulating the computer-executable pharmaceutical distribution model based at least in part on the defined scenario to obtain computational results; [iv] quantifying a value of a performance metric for the computational results; [v] defining a data set for the parameter; [vi] further calculating the time-varying behavior of the at least one parameter based on the historical data set to obtain backtesting computational results; [vii] further quantifying a value of the performance metric for the backtesting computational results; and [viii] adjusting a distribution constraint of the plurality of distribution constraints based on a difference between the computational results and the backtesting computational results or making the backtesting computational results accessible to a remote (for example third party) computing device.

Certain embodiments may provide, for example, a system for backtesting a pharmaceutical distribution model, the system comprising an analytic engine and one or more computing systems configured to perform operations comprising: [i] configuring a computer-executable pharmaceutical distribution model that comprises a plurality of distribution constraints; [ii] assigning at least one defined scenario for a parameter (or a plurality of parameters) of the computer-executable pharmaceutical distribution model; [iii] simulating the computer-executable pharmaceutical distribution model based at least in part on the defined scenario to obtain computational results; [iv] quantifying a value of a performance metric for the computational results; [v] defining a data set for the parameter; [vi] further calculating the time-varying behavior of the at least one parameter based on the historical data set to obtain backtesting computational results; [vii] further quantifying a value of the performance metric for the backtesting computational results; and [viii] adjusting a distribution constraint of the plurality of distribution constraints based on a difference between the computational results and the backtesting computational results or making the backtesting computational results accessible to a remote (for example third party) computing device.

The details of one or more embodiments of the invention are set forth in the accompanying drawings and the description below. Other features and advantages of the invention will become apparent from the description, the drawings, and the claims.

### BRIEF DESCRIPTION OF THE DRAWINGS

FIGS. 1A-B are diagrams depicting an example system for securing a pharmaceutical therapy.

FIGS. 2A-2B are exemplary graphical user interfaces depicting environments for distributing a pharmaceutical therapy.

FIGS. 3A-3B are exemplary graphical user interfaces depicting environments for distributing a pharmaceutical therapy.

FIGS. 4A-4C are flow charts depicting example processes for distributing a pharmaceutical therapy.

FIG. 5 shows a block diagram of an exemplary system in accordance with one or

more embodiments of the disclosure.

FIG. 6 depicts an exemplary interface of a negotiation engine provided to a distributor or distributor in accordance with one or more embodiments of the disclosure.

FIG. 7 depicts an exemplary interface of creating a new model space in the negotiation engine, in accordance with one or more embodiments of the disclosure.

FIG. 8 depicts an exemplary interface of a model space of the negotiation engine, in accordance with one or more embodiments of the disclosure.

FIG. 9 depicts an exemplary interface of specifying eligible patient population, in accordance with one or more embodiments of the disclosure.

FIG. 10 depicts an exemplary interface to begin a new negotiation in the negotiation engine, in accordance with one or more embodiments of the disclosure.

FIG. 11 depicts an exemplary interface presented to the distributor to begin a new negotiation, in accordance with one or more embodiments of the disclosure.

FIG. 12 depicts an exemplary interface of a “Treatment Duration Cap Rebate”, in accordance with one or more embodiments of the disclosure.

FIG. 13 depicts an exemplary interface of “Therapy Discontinuation Rebate”, in accordance with one or more embodiments of the disclosure.

FIG. 14 depicts an exemplary interface depicting a summary of the deal proposal before it is submitted to the pharmaceutical company, in accordance with one or more embodiments of the disclosure.

FIG. 15 depicts an exemplary interface of the negotiation engine provided to a pharmaceutical company, in accordance with one or more embodiments of the disclosure.

FIG. 16 depicts an exemplary interface presented to the pharmaceutical company to respond to a proposed negotiation, in accordance with one or more embodiments of the disclosure.

FIG. 17 depicts an exemplary interface for specifying a pricing strategy of the pharmaceutical company in response to the proposal from the distributor, in accordance with one or more embodiments of the disclosure.

FIG. 18 depicts an exemplary interface depicting a summary of the deal proposal before it is submitted to the distributor, in accordance with one or more embodiments of the disclosure.

FIG. 19 depicts an exemplary interface presented to the distributor during a negotiation, in accordance with one or more embodiments of the disclosure.

FIG. 20 depicts an exemplary interface that displays the pharmaceutical companies pricing model to the distributor, in accordance with one or more embodiments of the disclosure.

FIG. 21 depicts an illustrative flowchart of a process of a negotiation between two parties, in accordance with one or more embodiments of the disclosure.

FIG. 22 is a flow chart depicting an example process for distributing a pharmaceutical therapy.

FIG. 23 is a flow chart depicting an example process for testing a model for distributing a pharmaceutical therapy.

FIG. 24 depicts an optimization of a performance metric with respect to a parameter of a pharmaceutical therapy distribution constraint.

FIG. 25 depicts a series of scenarios generated with respect to a parameter of a pharmaceutical therapy distribution constraint.

FIG. 26 depicts calculation of a performance metric with respect to a scenario for a parameter.

Like reference numbers and designations in the various drawings indicate like elements.

### DETAILED DESCRIPTION

According to embodiments of the present disclosure, systems, computer program products, and associated computer-implemented methods are disclosed that accelerate complex decision-making processes between a number of organizations and incorporate elements of problem-solving, negotiation, and cooperation. In certain embodiments, for example, systems, computer program products, and associated computer-implemented methods may generate a pharmaceutical distribution model for a pharmaceutical therapy that meets certain performance criteria. The system may include or communicate with multiple, distributed computing systems. For example, a system may includes a supplier computing system for a supplier of the pharmaceutical therapy, a distributor computing system for a distributor computing system of the pharmaceutical therapy, and a third-party computing system the communicates with the both the supplier and the distributor computing systems.

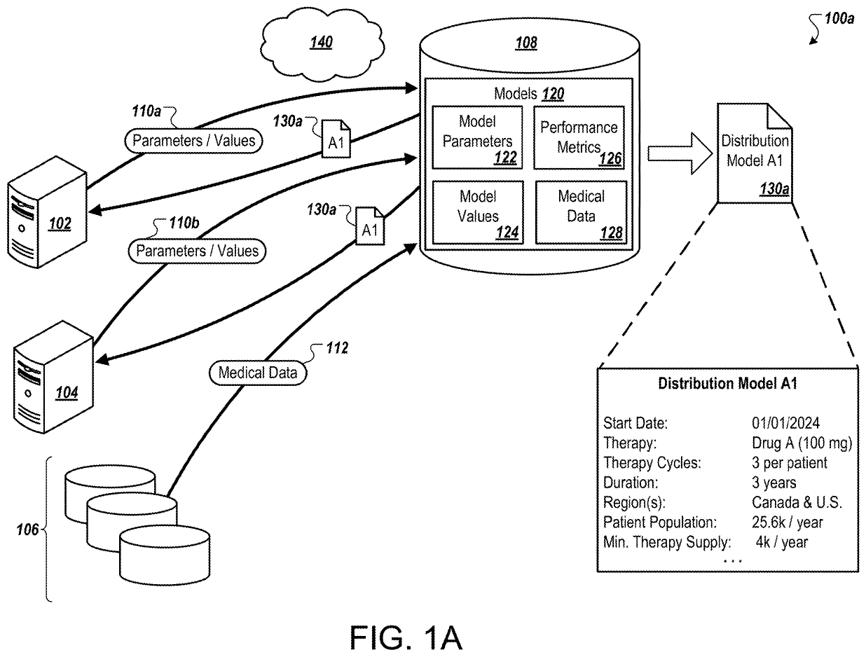

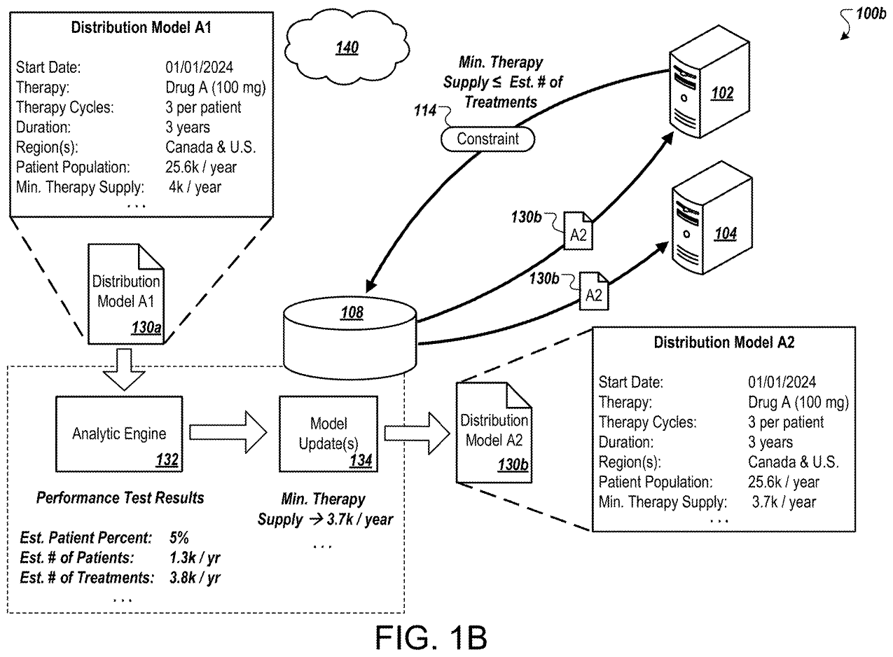

FIGS. 1A-1B are diagrams depicting an example system 100a-100b for securing a pharmaceutical therapy.

FIG. 1A is a diagram depicting an example system 100a for securing a pharmaceutical therapy. The system 100a includes a third-party computing system 108 for generating pharmaceutical distribution models 120 to distribute pharmaceutical therapies. The third-party computing system 108 communicates with a distributor computing system 102 of a distributor of a pharmaceutical therapy and a supplier computing system 104 of a supplier of the pharmaceutical therapy over a network 140. The system 100a can also communicate with different data sources 106 over the network 140. As described in more detail below, the third-party computing system 108 uses information, such as stored information and/or information obtained from external systems, to generate the pharmaceutical distribution models 120 for the distribution of pharmaceutical therapies.

The third-party computing system 108 includes one or more computers or computing devices. As an example, the third-party computing system 108 is a server system, such as an on-premises server system (for example, locally hosted), a cloud computing system, part of a cloud computing system, or the like.

The distributor computing system 102 includes one or more computers or computing devices. For example, the distributor computing system 102 is a server system, such as an on-premises server system (for example, locally hosted), a cloud computing system, part of a cloud computing system, or the like. A user of the distributor computing system 102 may access the distributor computing system 102 through a computing device, such as a desktop computer or a mobile device. After accessing the distributor computing system 102, the user is able to transmit information to the third-party computing system 108 over the network 140, view information transmitted by the third-party computing system 108 to the distributor computing system 102 over the network 140, and/or access the third-party computing system 108 over the network 140 (for example, access a sandbox environment hosted on the third-party computing system 108).

As another example, the distributor computing system 102 is computer or computing device accessible to one or more users of the distributor computing system 102 or a collection of computers or computing devices accessible to one or more users of the distributor computing system 102. In more detail, the distributor computing system 102 is or includes a desktop computer, a smart phone, a laptop computer, a tablet computer, or the like. Through the computer or computing device, a user of the distributor computing system 102 is able to transmit information to the third-party computing system 108 over the network 140, view information transmitted by the third-party computing system 108 to the distributor computing system 102 over the network 140, and/or access the third-party computing system 108 over the network 140 (for example, access a sandbox environment hosted on the third-party computing system 108). For example, through an employer smart phone, a user of the distributor computing system 102 is able to follow a link (for example, URL) to access a sandbox environment hosted on the third-party computing system 108 (for example, based on permissions through the use of the particular smart phone, after entering credentials for the particular Distributor and/or user, etc.).

The supplier computing system 104 include one or more computers or computing devices. For example, the supplier computing system 104 is a server system, such as an on-premises server system (for example, locally hosted), a cloud computing system, part of a cloud computing system, or the like. A user of the supplier computing system 104 may access the supplier computing system 104 through a computing device, such as a desktop computer or a mobile device. After accessing the supplier computing system 104, the user is able to transmit information to the third-party computing system 108 over the network 140, view information transmitted by the third-party computing system 108 to the supplier computing system 104 over the network 140, and/or access the third-party computing system 108 over the network 140 (for example, access a sandbox environment hosted on the third-party computing system 108).

As another example, the supplier computing system 104 is computer or computing device accessible to one or more users of the supplier computing system 104 or a collection of computers or computing devices accessible to one or more users of the supplier computing system 104. In more detail, the supplier computing system 104 is or includes a desktop computer, a smart phone, a laptop computer, a tablet computer, or the like. Through the computer or computing device, a user of the supplier computing system 104 is able to transmit information to the third-party computing system 108 over the network 140, view information transmitted by the third-party computing system 108 to the supplier computing system 104 over the network 140, and/or access the third-party computing system 108 over the network 140 (for example, access a sandbox environment hosted on the third-party computing system 108). For example, through an employer smart phone, a user of the supplier computing system 104 is able to follow a link (for example, URL) to access a sandbox environment hosted on the third-party computing system 108 (for example, based on permissions through the use of the particular smart phone, after entering credentials for the particular Supplier and/or user, etc.).

The network 140 is a communications network and can include one or more wired networks, wireless networks, or a combination of wired and wireless networks. For example, the network 140 is a local area network (LAN), a wide area network (WAN) such as the Internet, or includes a combination of one or more LANs and WANs. As another example, the network 140 is a cellular network.

In some implementations, the third-party computing system 108 communicates with multiple distributor computing systems and/or multiple supplier computing systems. For example, each pharmaceutical distribution model involves one or more suppliers of the corresponding pharmaceutical therapy and one or more distributors of the corresponding pharmaceutical therapy. Different models involve the same or different suppliers and/or distributors. As an example, a model 130 of the models 120 is for the distribution of Drug A in the U.S. and Canada, and involves a first supplier (for example, Supplier X) having the supplier computing system 104 and a first distributor (for example, Distributor X) having the distributor computing system 102.

As another example, a different model of the models 130 is for the distribution of pharmaceutical therapy in the U.S. and Canada that includes both Drug A and Drug B, and involves the Distributor X and multiple suppliers, such as the Supplier X to supply Drug A and a second supplier (for example, Supplier Y) to supply Drug B. In this example, in addition to communicating with the distributor computing system 102 and the supplier computing system 104, the third-party computing system 108 communicates with a third computing system (not shown) belonging to the second supplier.

As another example, a different model of the models 130 is for the distribution of Drug A in the U.S., Canada, and Mexico and involves the Supplier X to supply Drug A and multiple distributors, Distributor X to distribute the therapy in U.S. and Canada and a second distributor (for example, Distributor Y) to distribute the therapy in Mexico. In this example, in addition to communicating with the distributor computing system 102 and the supplier computing system 104, the third-party computing system 108 communicates with a third computing system (not shown) belonging to the second distributor.

In some implementations, the system 100a includes multiple distributor computing systems. For example, in generating a model for the distribution of a pharmaceutical therapy, the third-party computing system 108 receives information from a supplier computing system indicating that a first distributor is to distribute the treatment in a first region and that a second distributor is to distribute the treatment in a second region.

In some implementations, the system 100a includes multiple supplier computing systems. For example, a treatment may require a first pharmaceutical drug supplied by a first supplier and a second pharmaceutical drug supplied by a second supplier. In generating a model to distribute the treatment, the third-party computing system 108 communicates with a first supplier computing system, a second supplier computing system, and one or more distributor computing systems.

In some implementations, the distributor computing system 102 initializes the generation of a new model and provides information identifying one or more suppliers. For example, the information 110a described in more detail can include information identifying the supplier computing system 104.

In some implementations, the supplier computing system 104 initiates the generation of a new model and provides information identifying one or more distributors.

For example, the information 110b described in more detail can include information identifying the distributor computing system 102.

In some implementations, the system 100a includes the distributor computing system 102 and/or the supplier computing system 104. For example, the system 100a includes the distributor computing system 102, the supplier computing system 104, and the third-party computing system 108.

In some implementations, the one or more data sources 106 are external data sources accessible by the third-party computing system 108. The external data sources store medical data 112 that is accessible by the third-party computing system 108 and can include databases, websites, data sources accessible via a blockchain, or the like. The stored medical data 112 includes, for example, information related to different pharmaceutical therapies. As an example, the medical data 112 includes one or more of the following: information indicating a number of persons in different regions suffering from a condition that is treatable by a particular treatment, the number of treatments required per patient, time tables for patient treatment (for example, length of time from start to treatment until end of treatment, schedule for treatment cycles), insurance information (for example, indication of which, if any, insurances provided in the region cover the cost of the treatment or indication of the percent of persons in the region that are covered by insurance that covers all or part of the cost of treatment), statistical information (for example, percent of patients that recover from condition after treatment, percent of people suffering from a condition that is treatable by the particular treatment that seek treatment), trend information (for example, trends for the number of people suffering from a condition that is treatable by the particular treatment over time, trends for the effectiveness of the treatment over time, trends for the percent or number of people that seek the particular treatment over time, trend in cost of the treatment over time, etc.), or the like. The one or more data sources 106 may include, for example, local (for example, county), state, or country archives of medical data, medical insurance websites or databases, hospital websites or databases, websites or databases belonging to healthcare organizations, websites or databases of pharmaceutical suppliers (for example, website of a supplier for a treatment competing with a particular treatment provided by the pharmaceutical supplier corresponding to the supplier computing system 104), or the like.

In some implementations, the third-party computing system 108 obtains the medical data 112 from the one or more data sources 106 by sending a request for data to the one or more data sources 106 and, in response to the request, receiving the medical data 112.

In some implementations, the medical data 112 is for a particular treatment. For example, in generating the pharmaceutical distribution model 130 (for example, “Distribution Model A1”) for a pharmaceutical therapy involving the use of Drug A, the third-party computing system 108 transmits a request to the one or more data sources 106 for information related to the Drug A treatment (for example, for a particular dosage of Drug A considered for the distribution model 130, for a particular application of Drug A such as the use of Drug A in treating a Condition X, etc.). In response to transmitting the request, the third-party computing system 108 receives the medical data 112 in response.

In some implementations, the medical data 128 includes the medical data 112. For example, the medical data 112 obtained by the from the data sources 106 is stored by the third-party computing system 108 as part of the medical data 128 or is otherwise used by the third-party computing system to generate one or more models of the models 120 (for example, the distribution model 130).

In some implementations, the medical data 128 includes information obtained from one or more supplier and/or distributor computing systems. As an example, the medical data 128 includes a success rate in using Drug A to treat a Condition X obtained from the supplier computing system 104.

In some implementations, the third-party computing system 108 does not retain medical data after finalizing a distribution model. For example, after using the medical data 112, the third-party computing system 108 deletes the medical data 112. The third-party computing system 108 may do this to improve resource efficiency (for example, by freeing up data storage, reducing the amount of data storage needed, etc.) and/or so that the third-party computing 108 consistently obtains the most up-to-date information through the process of requesting new data from the one or more data sources, supplier computing systems, distributor computing systems, or the like when generating a new distribution model.

In some implementations, the system 100a includes the one or more of the data sources 106. As an example, the system 100a incorporates a database storing statistical information about the success rate and treatment time for a Drug A in the United States.

The third-party computing system 108 generates pharmaceutical distribution models 120 to distribute pharmaceutical therapies. Each pharmaceutical distribution model of the models 120 includes a set of pharmaceutical therapy distribution constraints that specify, for example, terms for the supply and distribution of a particular pharmaceutical therapy. The pharmaceutical therapy distribution constraints may be represented by one or more formulas, parameters (for example variables and/or coefficients such as fixed coefficients) of the model parameters 122, and/or values of the model values 124. For example, a first pharmaceutical therapy distribution constraint may be a represented by a variable representing a revenue amount minus a variable representing an expense amount must be greater than a static value (for example, a non-negotiable value such as a profit margin set greater than 0 indicating that the cost of the deal cannot be greater than the anticipated profit from the deal, etc.); a second pharmaceutical therapy distribution constraint may be represented by a parameter having a vector of values or a vector of assignable values, or a formula represented by one or more parameters and one or more values. In more detail, one pharmaceutical therapy distribution constraint may be a formula that represents the estimated number of treatments required per year. In this example, the formula includes a first parameter component that represents the number of anticipated patients per year multiplied by a constant value component that represents the number of treatments per patient per year.

The model parameters 122 include multiple, different parameters (for example a superstructure of parameters) for defining various pharmaceutical distribution models 120. As an example, these parameters can include one or more of the following: a parameter indicating a region where a pharmaceutical therapy is to be distributed, a parameter indicating a supplier or manufacturer for a pharmaceutical therapy, a parameter indicating an anticipated patient population for a pharmaceutical therapy, a parameter indicating a number of persons or potential patients suffering from a condition treatable by a treatment, a parameter indicating a number of treatments required to treat a patient in general for a particular condition, a parameter indicating a time of treatment in general or for a particular condition, a parameter indicating the term (for example length, or number of pharmaceutical therapys) of an agreement, one or more parameters indicating financial information for securing treatment (for example, cost to obtain supply of treatment, pricing tiers for treatment based on number of patients or number of treatments, rebates based on number of patients or number of treatments, etc.), a parameter indicating one or more competing treatments to a particular treatment, a parameter(s) indicating one or more existing distributors of a treatment (for example, for a particular region), a parameter indicating a success rate for the treatment in general or for a particular condition, a parameter indicating a particular dosage of the treatment, a parameter indicating a treatment name (for example, brand name, generic name, and/or compound name for a pharmaceutical drug), a parameter indicating whether a treatment includes multiple pharmaceutical drugs, or the like.

Within the third-party computing system 108, one or more values of the model values 124 are associated with one or more parameters of the model parameters 122. For example, a parameter indicating a region where a pharmaceutical therapy is to be distributed may point to a list of country codes that are part of the model values 124, or to multiple country codes for each potential country that are part of the model values 124.

In some implementations, a model parameter of the model parameters 122 references (for example may be equated to, may be greater than, or may be less than) a different parameter or combination of parameters. Such a parameter may be a pharmaceutical therapy distribution constraint. For example, a model parameter for the anticipated number of patients per year for a particular treatment can reference a first parameter for the number of eligible patients for the treatment, a second parameter for the percent of persons suffering from a condition or particular condition treatable by the treatment that seek treatment (for example, within the particular region considered), and a third parameter for the anticipated distribution share for the distributor (for example, within the particular region considered, such as 100% if there are no other distributors of the treatment in the region). In this example, the parameter for the anticipated number of patients is equated to the first parameter multiplied by the second and third parameters. Alternatively, the pharmaceutical therapy distribution constraint may comprise an inequality in which the parameter for the anticipated number of patients is less than or equal to the first parameter multiplied by the second parameter.

In some implementations, the term eligible patients refers to persons in a particular region where the treatment is to be distributed that have a condition or particular condition treatable by the treatment (for example, a condition treatable by the dosage of treatment considered for distribution). An eligible patient may also need to meet other requirements, such as one or more of the following: be at least a certain age (for example, based on the treatment or dosage of the treatment considered), be below a certain age (for example, based on the treatment or dosage of the treatment considered), be within a particular age range (for example, based on the treatment or dosage of the treatment considered), have insurance that covers all or part of the treatment, not be taking a pharmaceutical drug that has been identified to be incompatible with the treatment (for example, as a result of drug interaction), not have a condition that is incompatible with the treatment (for example, not have an autoimmune disease should the treatment increase health risk for those with an autoimmune disease), or the like. Continuing the preceding example, an parameter associated with the eligibility of a patient can itself reference one or more other parameters, such as a first parameter for the total number of persons in a region diagnosed with a condition or particular condition treatable by the treatment, a second parameter for the percent of persons in the region that are adults if the dosage considered for the treatment is for adults only, and a third parameter for the percent of persons in the region that are not taking a pharmaceutical drug incompatible with the treatment. In this example, the eligible patient parameter is represented by the first parameter multiplied by the second and third parameters.

The model values 124 are assigned to correspond to at least a portion of the model parameters 122 automatically or based on information received from one or more external sources. As an example, for a parameter indicating the success rate of a Drug A in treating a Condition X, the third-party computing system 108 automatically calculates a value based on statistical data on the success rate of Drug A in treating Condition X obtained from one of the data sources 106 and based on success rate provided by the supplier computing system 104. The success rate provided by the supplier computing system 104 may be determined at the supplier computing system 104 using, for example, confidential statistical data indicating the success rate of Drug A in treating Condition X.

As an example, in a simulation mode, a parameter can be assigned a first value in a first scenario and a second (possibly different value) in a second scenario. As another example, a parameter can be assigned a value based on (for example derived from) information obtained from the distributor computing system 102 and/or the supplier computing system 104. As another example, a parameter can be assigned a value based on information received from an external source. Additionally or alternatively, assigning a value or values to a particular parameter includes assigning a value or set of values to the particular parameter as a set (or fixed) value (for example, based on a selection made by a user of the distributor computing system 102 and/or the supplier computing system 104).

In some implementations, the third-party computing system 108 first establishes a parameter of the model parameters 122 and then a set of one or more possible values of the model values 124 that are assignable to the parameter. If there are multiple possible values assignable to the parameter, the third-party computing system 108, in generating or adjusting a particular pharmaceutical distribution model of the models 120, can assign one or more of the possible values to the parameter (for example, based on a selection made by a user of the distributor computing system 102, a selection made by a user of the supplier computing system 104, values assigned to other parameters of the particular pharmaceutical distribution model, etc.).

Each pharmaceutical therapy distribution constraint of a model of the models 120 may be represented in whole or in part by a matrix. Additionally or alternatively, a scenario is represented by a matrix and a pharmaceutical therapy distribution constraint refers to one or more values in the matrix for the scenario. The matrix may be square or the matrix may be non-square (i.e., different rows may have differing numbers of columns). Each row of the matrix can correspond to a parameter of the model parameters 122 and each value in the matrix can be a value of the model values 124. If the pharmaceutical therapy distribution constraint is represented as a formula, the formula can include references (for example, pointers) to locations within a matrix for the pharmaceutical therapy distribution constraint. As an example, each row of a matrix for a particular pharmaceutical therapy distribution constraint corresponds to a different parameter and the vector of one or more values in a given row represents the assigned values for the corresponding parameter or the possible values assignable to the corresponding parameter. In this example, a first row corresponds to a first parameter of the model parameters 122, and a second row corresponds to a second parameter of the model parameters 122. The first row is a first vector of one or more values from the model values 124 corresponding to the first parameter and the second row is a second vector of one or more values from the model values 124 corresponding to the second parameter.

As an example, for a pharmaceutical therapy distribution constraint for a purchase price per year for the distribution of a particular treatment, the third-party computing system 108 generates a matrix for the pharmaceutical therapy distribution constraint as part of generating the model 130 for the Drug A treatment. The pharmaceutical therapy distribution constraint (for example, Constraintppy) can be represented as an equation for the anticipated number of patients per year (for example, parameter x) multiplied by the number of treatments per patient per year (for example, parameter y) and the cost per treatment (for example, parameter z). The anticipated number of patients per year references a first row of the matrix that includes a first value (for example, potentially the only value in the row), such as 3500 to indicate that there is anticipated 3500 patients per year that will be treated with the treatment. The number of treatments per patient per year references a second row of the matrix that includes a first value (for example, potentially the only value in the row), such as 30 to indicate that a typical patient receives 30 doses or cycles of the treatment per year. The cost per treatment references a third row of the matrix for a third parameter that includes multiple values (for example, for different pricing tiers based on the number of patients, the number of treatments, etc.). For example, a first value in the third row is 1500 indicating a cost of $1500 per treatment and is applicable to the first 1000 patients, a second value in the third row is $1200 per treatment and is applicable to the next 2000 patients, and a third and final value in the third row is $1000 per treatment and is applicable to any remaining patients. Accordingly, as currently defined in this example, the pharmaceutical therapy distribution constraint for a purchase price per year for the distribution of a particular treatment is $132,000,000 per year. An example representation of the purchase price per year pharmaceutical therapy distribution constraint and the matrix for the purchase price per year pharmaceutical therapy distribution constraint are shown below:

$${Constraint}_{PPY} ( x , y , z ) ; [ \begin{matrix}x \\ y \\ z\end{matrix} ] = [ \begin{matrix}3500 & & \\ 30 & & \\ 1500 & 1200 & 100\end{matrix} ]$$

Various modifications to simplify or increase the complexity of a pharmaceutical therapy distribution constraint are possible. Continuing the preceding example, the formula representing the pharmaceutical therapy distribution constraint for a purchase price per year for the distribution of a particular treatment can include additional parameters to take into account the total number of treatments required per patient, an anticipated increase in the number or percentage of patients per year, or the like. The values referenced to calculate this pharmaceutical therapy distribution constraint and/or the parameters included in this pharmaceutical therapy distribution constraint are adjustable by, for example, information received from the distributor computing system 102, the supplier computing system 104, or new information obtained from one or more of the data sources 106. For example, as described in more detail below, using a sandbox environment provided through the third-party computing system 108, users of the distributor computing system 102 and the supplier computing system 104 are able to adjust this pharmaceutical therapy distribution constraint and, therefore, a model being developed for this treatment through one or more rounds of negotiation that can quickly take place through the sandbox environment.

Alternatively, the third-party computing system 108 may determine different values to assign to a particular parameter for different simulation scenarios based on the model values 124. In determining different values, the third-party computing system 108 may randomly or pseudo-randomly assign values to parameters. For example, a first model value may specify a mean value for a parameter and a second model value may specify a standard deviation for the parameter. Using the first and second model values, a performance simulation may be based on a plurality of scenarios in which the parameter is assigned randomly or pseudo-randomly according to the mean and standard deviation of the parameter specified by the first parameter and the second parameter.

Continuing the earlier example, the third-party computing system 108 can calculate a mean value of 57% for a parameter that indicates the likelihood of a patient in the U.S. seeking treatment for a condition that is treatable with Drug A, and one standard of deviation for the same parameter as a range of 48% through 64%. The third-party computing system 108 may then generate different scenarios using these values. As an example, the third-party computing system 108 may generate the following scenarios: a first scenario where a value of the parameter is set to the mean value of 57%, a second scenario where the parameter is set to a randomized or pseudo-randomized value between the lower-end of the range of 48% and the mean of 57%, a third scenario where the parameter is set to a randomized or pseudo-randomized value between 52.5% (for example, halfway between the lower-end of the range specified by one standard deviation and the mean) and the mean of 57%, a fourth scenario where the parameter is set to a randomized or pseudo-randomized value between the mean of 57% and the upper-end of the range of 64%, and a fifth scenario where the parameter is set to a randomized or pseudo-randomized value between the mean of 57% and 60.5% (for example, halfway between the mean and the upper-end of the range specified by one standard deviation). As another example, the third-party computing system 108 may generate all five scenarios by setting the parameter to a randomized or pseudo-randomized value between 48% and 64%. As another example, the third-party computing system 108 may generate a set of ten values randomized or pseudo-randomized between 48% and 64% and then select a subset of five values from the set of ten values that are closest in distance to the mean of 57%.

As another example, different scenarios for testing and generating the model 130 can include alternative pharmaceutical therapies. For example, a first scenario for the model 130a includes a treatment set to the 100 mg dosage of Drug A, a second scenario for the model 130a includes a treatment set to a 80 mg dosage of Drug A that is used to treat the same medical conditions (for example, is cheaper than the 100 mg dosage but slightly less effective than the 100 mg dosage), a third scenario for the model 130a includes a treatment of 50 mg of Drug B that is used to treat one or more of the same medical conditions as the other treatments, and a fourth scenario for the model 130a includes a treatment of 7 mg of Drug C that is used to treat one or more different medical conditions. Each of the treatments can be supplied by the supplier having the supplier computing system 104 such that the model 130a contemplates one supplier. Alternatively, two or more of the different pharmaceutical therapies can be supplied by different suppliers such that the model 130a contemplates two or more different suppliers. In this example, the finalized model 130 can include a single supplier that is not necessarily the supplier having the supplier computing system 104. Alternatively, the finalized model 130 can include multiple suppliers such as a supplier having the supplier computing system 104 and a second, different supplier.

As another example, the third-party computing system 108 may determine a value to assign to a particular parameter using one or more statistical functions performed on a subset of the model values 124. As an example, in determining a value to assign to the parameter that indicates the likelihood of a patient in the U.S. seeking treatment for a condition that is treatable with Drug A, the third-party computing system 108 can calculate a current likelihood based on a set of prior values within a threshold time period (for example, from within a year of the current date, six months of the current date, a month of the current date, etc.) indicating the number of persons in the U.S. that sought treatment, use the set of prior values and/or additional prior values to determine a trend in the likelihood of a patient in the U.S. seeking treatment for a condition that is treatable with Drug A, and applying the trend to the current likelihood to determine an anticipated likelihood at the start date for the distribution of Drug A (for example, Jan. 1, 2024) or an average likelihood over the duration of the agreement (for example, from Jan. 1, 2024 through Jan. 1, 2027). In more detail, the third-party computing system 108 can calculate the mean of the prior 8 months of data to find that the current likelihood is 57%, analyze the last three years of data to determine that the likelihood is anticipated to increase at a rate of 2.3% per year, and use the previous information to determine that the likelihood at the start date is 57.66% (for example, six months from the current date) and the average likelihood over the course of the agreement is 59.39%. The third-party computing device 108 may then assign a value of 0.5939 to the parameter.

In some implementations, one or more values to be assigned to a parameter are provided to the third-party computing system 108. For example, the supplier computing system 104 may calculate a value of 60% for the parameter that indicates the likelihood of a patient in the U.S. seeking treatment for a condition that is treatable with Drug A based on internal data that may be confidential. Instead of providing confidential information, the supplier computing system 104 may provide the third-party computing system 104 the value instead of the raw data.

In some implementations, if there is a dispute or discrepancy between a value to be assigned to a particular parameter, the third-party computing system 104 can take one or more of the following actions: select one value over another (for example, values calculated or generated by the third-party computing system 108 are preferred over values supplied by supplier computing system 104 or the distributor computing system 102, prefer values based on indication of the size or quality of data that the value is calculated off of, etc.), average the different values, determine a new value based on the different values such as by applying a first weight to a first value, a second weight to a second value, and averaging the weighted values (for example, where weights are determined based on preferences for value sources and/or indications of data quality used to calculate values), etc.

In some implementations, a pharmaceutical therapy distribution constraint is a function of a particular scenario or is defined for a particular scenario. A pharmaceutical therapy distribution constraint for a first scenario might differ from the same pharmaceutical therapy distribution constraint for a second scenario in the formula used to represent the pharmaceutical therapy distribution constraint, the parameters referenced by the pharmaceutical therapy distribution constraint, and/or the values set for the parameters or otherwise referenced by the pharmaceutical therapy distribution constraint. During the process of generating the model 130, the third-party computing system 108 can start building the model using an initial scenario. Such a scenario can be determined based on finalized scenario(s) of one or more previously generated models, the information 110a, the information 110b, and/or the medical data 112. As will be described in more detail below with respect to FIG. 1B, the third-party computing system 108 can update the scenario and, therefore, the model 130 based on performance results, for example, to optimize the model 130. As another example, the third-party computing system 108 can generate a set of additional scenarios for testing as described above.

As an example, a pharmaceutical therapy distribution constraint on the anticipated number of treatments X in a particular scenario S1 (X_{S1}) may be X_{S1}(x, t)=x₁+x₂·max{0, t−1}, where x₁ representing the anticipated number of treatments for the first year t=0 and x₂ representing the anticipated number of treatments per year for any subsequent years t. Alternatively, the pharmaceutical therapy distribution constraint might be an inequality rather than an equality. For example, the pharmaceutical therapy distribution constraint could be X_{S1}(x, t)≤x₁+x₂·max{0, t−1}.

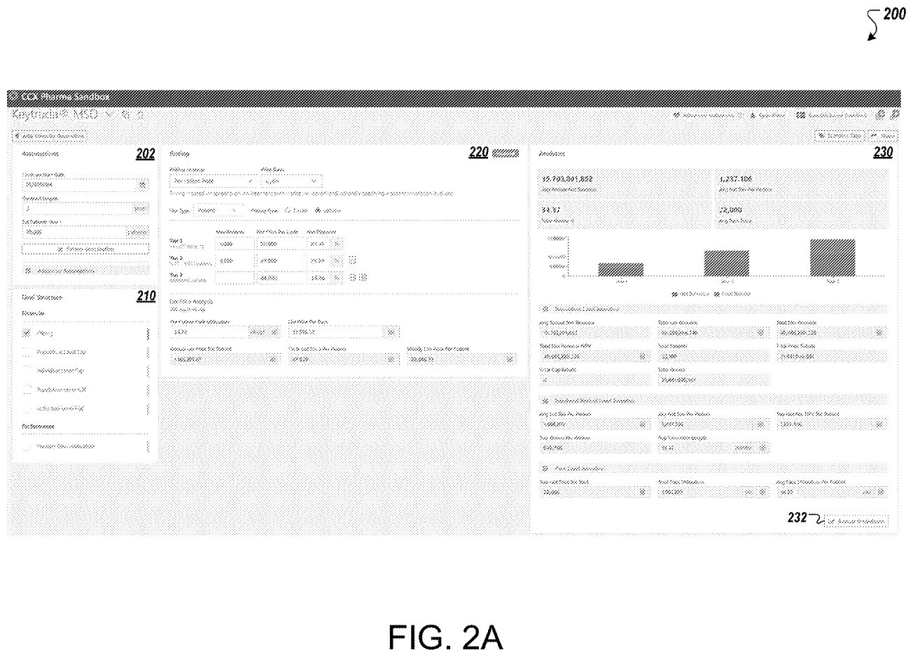

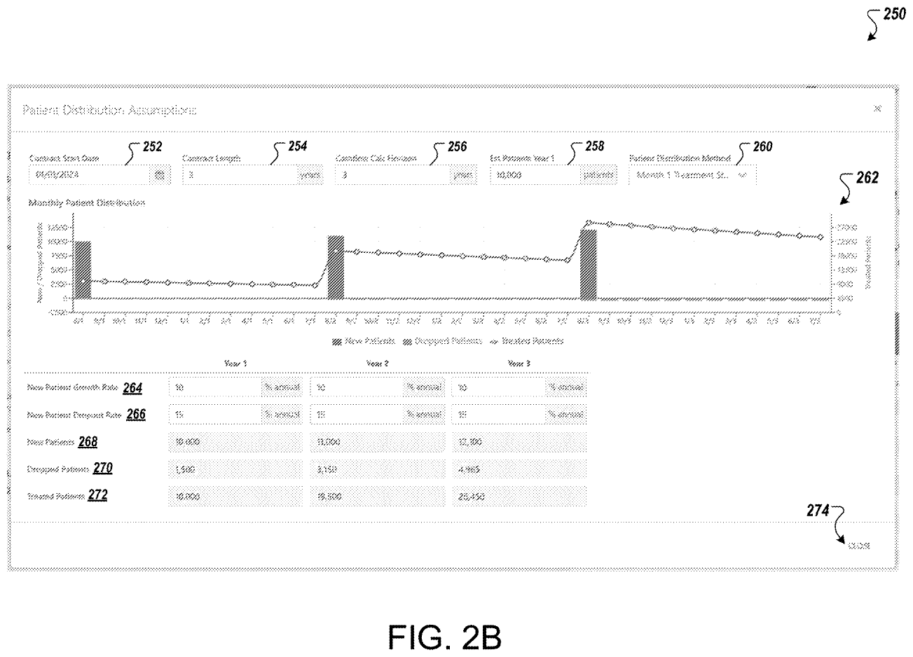

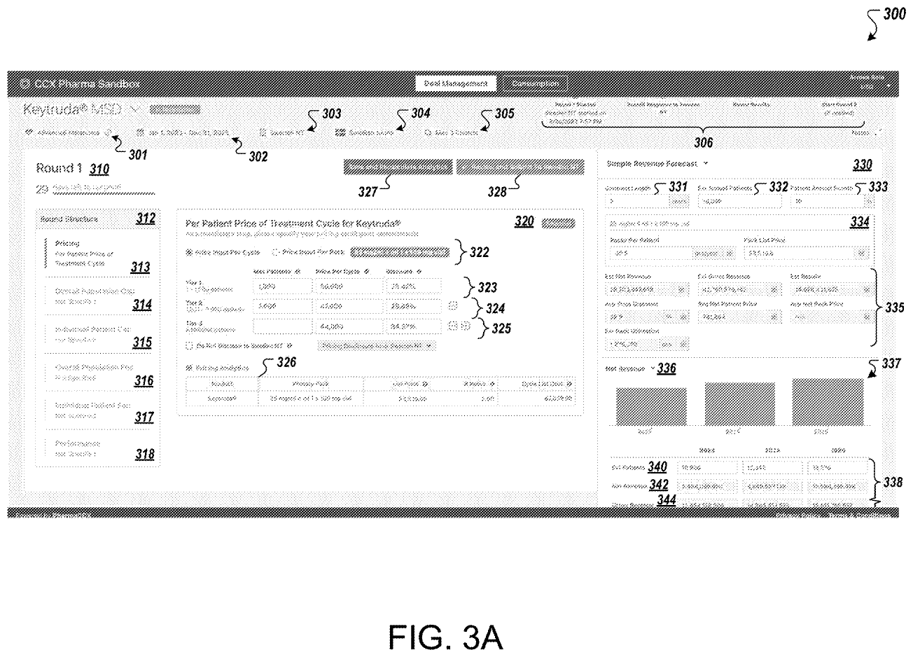

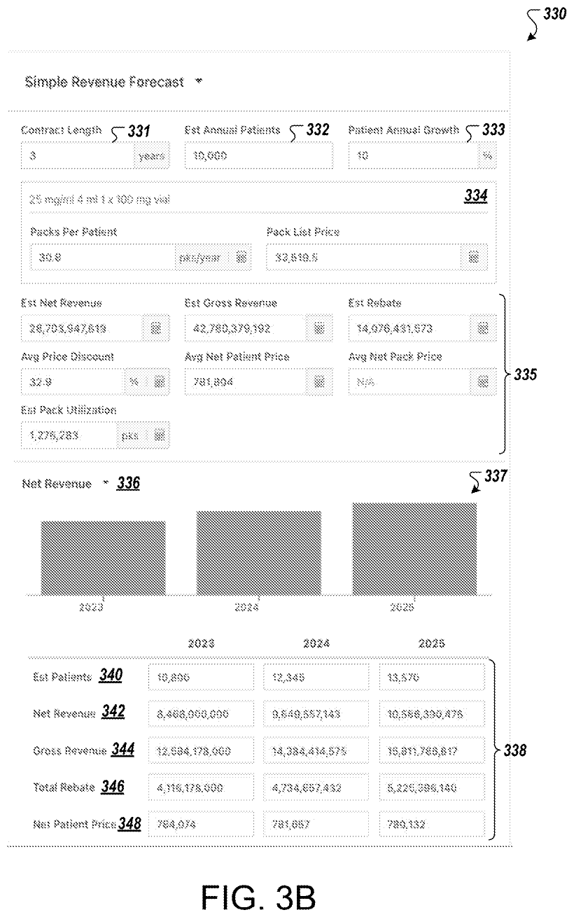

In the process of generating the pharmaceutical distribution model 130, a user of the distributor computing system 102 or a user of the supplier computing system 104 interacts with the third-party computing system to start the process of generating a new pharmaceutical distribution model. As an example, the third-party computing system 108 provides a computing environment (for example a sandbox environment) that provides one or more user interfaces, such as the user interface 200 shown in FIG. 2A, the user interface 250 shown in FIG. 2B, the user interface 300 shown in FIG. 3A, and/or the user interface 350 shown in FIG. 3B and as described in more detail below. The one or more user interfaces can include an initial user interface that allows a user to make a selection to generate a new pharmaceutical distribution model. For example, an interface area 202 of the user interface 200 shown in FIG. 2A includes a number of interactive fields where a user can input values for different parameters (for example, model assumptions) for a new pharmaceutical distribution model. As another example, another interface area 220 of the user interface 200 shown in FIG. 2A includes various interface elements for different pricing parameters, such as drop-down menu that a user can interact with to select a pricing strategy (for example, a per patient price strategy), a check box or toggle element that a user can interact with to select a pricing type (for example, tiered or volume), or the like. The initial user interface may request information or other selections from the user, such as a selection of a particular treatment or set of treatments for the new model, one or more distributors for the treatment, one or more suppliers of the treatment, or the like. After making such a selection, the third-party computing system 108 can provide the user with an interactive interface, through which the user can set the initial pharmaceutical therapy distribution constraints, parameters, and/or values for the model 130, such as the user interface 200 shown in FIG. 2A.

In some implementations, the third-party computing system 108 provides a user the option to generate a new pharmaceutical distribution model based on a previous, pre-existing, or default pharmaceutical distribution model. As an example, after providing instructions to generate new pharmaceutical distribution model (for example, by making a selection through a user interface of a computing environment provided by the third-party computing system 108), the third-party computing system 108 presents on a user interface of a sandbox computing environment a list of previous models of the models 120 that were previously generated for the supplier computing system 104 or previously used by the Supplier A (for example, supplier having the supplier computing system 104). The presented list may be further limited to, for example, only those models where the Distributor A (for example, distributor having the distributor computing system 102) was a party (for example, was a distributor), only those models that included the same or similar treatment (for example, same pharmaceutical drug but different dosage), or the like.

In some implementations, the third-party computing system 108 recommends one or more previously generated models to use as a base for a new pharmaceutical distribution model. For example, the third-party computing system 108 performs a search of models in the models 120 that involves the same supplier, distributor, treatment, or the like and presents the user with a list of the recommended models. The third-party computing system 108 may rank the models in the list based from the most relevant to the least relevant. For example, in generating a list of recommended models for a user of the supplier computing system 104 for a Drug A treatment with a dosage of 100 mg, the third-party computing system 108 may generate a list where the first recommended model of the models 120 is a model for a Drug A treatment with a dosage of 100 mg in a different region from the current region, the second recommended model is a model for a Drug A treatment with a dosage of 80 mg for the same region as the current region and the same distributor, a third recommended model is a model for a Drug A treatment with a dosage of 80 mg for the same region as the current region and a different distributor, a fourth recommended model is a model for a Drug A treatment with a dosage of 80 mg for a different region, and a fifth recommended model is a model for a Drug B treatment with a dosage of 10 mg for the same region and the same distributor.

In the process of generating the pharmaceutical distribution model 130, the distributor computing system 102 transmits information 110a to the third-party computing system 108 and the distributor computing system 104 transmits information 110b to the third-party computing system 108. The information 110a and the information 110b includes, for example, information related to one or more parameters or values of the model 130. For example, the information 110a and/or the information 110b includes one or more of the following: an addition of a pharmaceutical therapy distribution constraint to the model 130 that references one or more parameters, a removal of a pharmaceutical therapy distribution constraint to the model 130 that referenced one or more parameters, a modification to a pharmaceutical therapy distribution constraint of the model 130, an assignment of a value to a parameter of the model 130, information that can be used by the third-party computing system 108 to calculate a value to assign to a parameter of the model 130, a selection or approval of one or more recommendations made by the third-party computing system 108 (for example, recommended value to assign to a parameter, recommended formula to assign to a pharmaceutical therapy distribution constraint, recommended previously generated model to base new model on, etc.). The information 110a and/or the information 110b can additionally or alternatively include other information, such as a number of rounds for modifying the pharmaceutical distribution model 130 (for example, maximum of three rounds to negotiate the model 130), an acceptable range of rounds for modifying the pharmaceutical distribution model 130 (for example, between two and four rounds), an indication of one or more distributors of the treatment associated with the model 130 (for example, 100 mg of Drug A), an indication of one or more suppliers of the treatment associated with the model 130, or the like.

The third-party computing system 108 can generate the model 130 based on stored or received information. For example, the third-party computing system 108 can generate the model 130 based on the information 110a, the information 110b, the medical data 112, previously stored information, or a combination thereof. As an example, the third-party computing system 108 generates the model 130a (for example, initial version of the model 130) based on the combination of the information 110a, the information 110b, and the medical data 112.

In generating the model 130, the third-party computing system 130 can define a set of pharmaceutical therapy distribution constraints for the model 130, assign a set of values to parameters for the model 130, or both. For example, the model 130a includes a start date parameter with an assigned value of Jan. 1, 2023, a treatment parameter with an assigned value of a Drug A at 100 mg, a duration parameter with an assigned value of three (3) years, a region(s) parameter with an assigned value of Canada and United States (or CA and US), a patient population parameter with an assigned value of 25,600 patients per year, and a minimum treatment supply of 4,000 units per year and an associated pharmaceutical therapy distribution constraint. The model 130a can include various additional parameters with assigned values and/or pharmaceutical therapy distribution constraints.

In some implementations, the third-party computing system 108 generates multiple, different pharmaceutical therapy distribution constraints and/or assigns different possible values to parameters for a particular model. For example, the third-party computing system 108 can generate five different scenarios for the model 130a. Each scenario may differ from the other in that one or more pharmaceutical therapy distribution constraints are defined differently for a particular scenario when compared to that of a different scenario, a different set of values is assigned to the model parameters for a particular scenario when compared to that of a different scenario, or a combination of the two. As an example, the displayed values for parameters of the model 130a may represent a first of multiple scenarios generated by the third-party computing system 108.

The model 130a shown in FIG. 1A can represent the initial version of the model parameter values set by the party initializing the model 130). For example, the values assigned to the parameters of the model 130a may be initial values generated by the third-party computing system 108 and/or supplied by the initializing party before the third-party computing system 108 receives any feedback from a counterparty (for example, the distributor computing system 102 if the supplier initialized the model 130, or the supplier computing system 104 if the distributor initialized the model 130).

Alternatively, the model 130a can represent a version of the model 130 after one or more rounds of negotiation. For example, the values assigned to the parameters of the model 130a can be those approved or adjusted by the distributor computing system 102 after a first round of negotiation when the supplier computing system 104 initialized the generation of the model 130.

After generating the distribution model 130a, the third-party computing system 108 can make the model 130a available to the distributor computing system 102, the supplier computing system 104, or both. For example, the third-party computing system 108 can transmit a data object that represents the model 130a to the distributor computing system 102 and the supplier computing system 104. Each of the systems 102 and 104 can then execute the data object on their respective systems to run the model 130a. As another example, the third-party computing system 108 can display or execute the model 130a on the third-party computing system 108 and permit a user of the distributor computing system 102 and/or a user of the supplier computing system 104 to access the displayed or executed model through a URL.

In some implementations, the third-party computing system 108 enforces a set of one or more permissions. These permissions can control the information that each of the parties can access and may be set in whole or in part by the distributor computing system 102, the supplier computing system 104, the third-party computing system 108, or a combination thereof. As an example, the model 130a can include a parameter that indicates the anticipated profit per year for the supplier based on proprietary information indicating how much the supplier spends on purchasing or manufacturing the 100 mg dosage of Drug A. This section or portion of the model 130a may only be made accessible to the supplier computing system 104. As another example, the distributor may have supplied information indicating that they anticipate using 3,500 treatments per year with the 100 mg dosage of Drug A in the CA and US regions but included a request (for example, as part of the information 110a) for this value not to be shared with the supplier computing system 104 and/or that only an indication be shared with the supplier computing system 104 indicating whether the set value for the minimum treatment supply parameter (for example, initially set by the supplier) is less than the anticipated number of treatments per year. Through these techniques, the distributor and/or the supplier can limit the information shared with their respective counterparty through the process of generating the pharmaceutical distribution model 130.

FIG. 1B is a diagram depicting an example system 100b for securing a pharmaceutical therapy. The system 100b includes the third-party computing system 108 that communicates with the distributor computing system 102 of a distributor of a pharmaceutical therapy and the supplier computing system 104 of a supplier of the pharmaceutical therapy over the network 140. The system 100b can also communicate with the different data sources 106 shown in FIG. 1A over the network 140. In some implementations, the system 100b is the system 100a shown in FIG. 1A. As described in more detail below, after generating the pharmaceutical distribution model 130a, the third-party computing system 108 analyzes the model 130a and updates the model 130a to generate a model 130b, an updated version of the model 130a.

After generating the pharmaceutical distribution model 130a, the third-party computing system 108 can use an analytic engine 132 to analyze the model 130a. The analytic engine 132 can include one or more programs designed to analyze pharmaceutical distribution models. The analytic engine 132 may analyze the pharmaceutical distribution model 130a by, for example, dynamically simulating the model 130a. As an example, the analytic engine 132 can perform a simulation for each of multiple scenarios for the model 130a (for example, generated by the analytic engine 132 of the third-party computing device 108). A simulation may indicate, for example, a performance of the model 130a through a set of performance metrics. These performance metrics may indicate pharmaceutical therapy distribution constraint violations. In more detail, the performance metrics can include, for example, a metric indicating whether a pharmaceutical therapy distribution constraint of the model 130a is met or not, a metric indicating whether a pharmaceutical therapy distribution constraint of the model 130a is met or not over time (for example, through the duration of the distribution agreement), a metric indicating the distance (for example, Euclidean distance) between a first pharmaceutical therapy distribution constraint of the model 130a (for example, indicating anticipated performance in one area such as the anticipated number of patients over time) and (i) a second pharmaceutical therapy distribution constraint of the model 130a (for example, indicating a desired or minimum performance in one area such as the minimum number of patients over time) or (ii) one or more predetermined values (for example, vector of values indicating a desired or minimum number of patients at different points in time), a metric indicating whether a first pharmaceutical therapy distribution constraint of the model 130a is within a threshold (for example, threshold value, threshold percentage, etc.) from (i) a second pharmaceutical therapy distribution constraint of the model 130a or (ii) one or more predetermined values, a metric indicating a likelihood that a pharmaceutical therapy distribution constraint of the model 130a will be met or not, a metric indicating a likelihood that a pharmaceutical therapy distribution constraint of the model 130a will be met or not over time (for example, through the duration of the distribution agreement), etc. The performance metrics can additionally or alternatively include metrics based on multiple pharmaceutical therapy distribution constraints, such as, as a metric indicating an overall performance of the model 130a for a particular scenario across all pharmaceutical therapy distribution constraints or a select subset of pharmaceutical therapy distribution constraints (for example, pharmaceutical therapy distribution constraints that a user of the distributor computing system 102 and/or of the supplier computing system 104 has indicated belong to a set of more relevant pharmaceutical therapy distribution constraints, pharmaceutical therapy distribution constraints that the third-party computing system 108 determines are most often negotiated or disputed per its past models or stored data, etc.), an overall performance of the model 130a over time for a particular scenario across all pharmaceutical therapy distribution constraints or a select subset of pharmaceutical therapy distribution constraints, etc.

As an example, in performing a simulation of the model 130a, the analytic engine 132 may use the assigned values of parameters of the model 130a to simulate the performance of the model over time (for example, over the three-year duration of the agreement for the distribution of 100 mg of Drug A starting on Jan. 1, 2024). In more detail, the analytic engine 132 may simulate the anticipated number of eligible patients over time. The analytic engine 132 can use, for example, (i) a pharmaceutical therapy distribution constraint for calculating the number of eligible patients over time and (ii) different sets of values assigned to the parameters of the pharmaceutical therapy distribution constraint that correspond to different scenarios to simulate the number of eligible patients that the distributor can expect over time for each of the different scenarios.

In some implementations, the analytic engine 132 simulates the model 130a using randomized or pseudo-randomized analysis techniques. For example, the analytic engine 132 performs one or more Monte Carlo simulations by randomly sampling from subsets of data in the medical data 128 corresponding to different pharmaceutical therapy distribution constraints. In more detail, the analytic engine 132 can identify from the medical data 128 a first subset of data (for example, can generate a data set from the medical data 128) indicating the number of persons in U.S. diagnosed with a condition that is treatable by 100 mg of Drug A over the past five years and a second subset of data indicating the number of persons in Canada diagnosed with a condition that is treatable by 100 mg of Drug A over the past five years. In performing the one or more Monte Carlo simulations to, for example, simulate the anticipated number of treatments required over time, the analytic engine 132 can randomly sample from the first subset of data and the second subset of data. Each sample combination can represent a different scenario that is being simulated.

In some implementations, the analytic engine 132 uses the results from simulating the model 130a to calculate performance metrics for the model 130a. As an example, after simulating the model 130a, the analytic engine 132 uses the simulation results to calculate one or more performance metrics (for example, one or more performance metrics for the corresponding parameter value(s), for a particular pharmaceutical therapy distribution constraint, for the model 130a as a whole for a particular scenario, for the model 130a as a whole across multiple scenarios, etc.).

In some implementations, the pharmaceutical therapy distribution constraints include performance rules. These rules may be set such that they are not adjustable. The performance rules can be provided by the supplier computing system 104, the distributor computing system 102, and/or the third-party computing system 108. As an example, the distributor computing system 102 transmits a pharmaceutical therapy distribution constraint 114 to the third-party computing system 108 that the third-party computing system 108 uses to analyze and/or update the model 130a. The pharmaceutical therapy distribution constraint 114 can be a performance rule, such as a rule that the minimum number of treatments supplied by the supplier must be less than or equal to the estimated number of treatments. In evaluating the performance of the model 130a, the analytic engine 132 can generate a performance metric based on a comparison of the performance results from one or more simulations of the model 130a to the pharmaceutical therapy distribution constraint 114 that indicates, for example, whether the model 130a met the rule, did not meet the rule, a likelihood of success in the model 130a meeting the rule, a likelihood of failure in the model 130a meeting the rule, a score indicating how often the model 130a met or did not meet the rule for different scenarios, etc.

For example, the simulation results from simulating the model 130a can include results indicating the anticipated number of treatments of the 100 mg dosage of Drug A required over time. The performance results may indicate, for example, that an average of 4500 treatments are anticipated per year under a first scenario, an average of 4000 treatments are anticipated per year under a second scenario, an average of 3700 treatments are anticipated per year under a third scenario, an average of 3500 treatments are anticipated per year under a fourth scenario, and an average of 3300 treatments are anticipate per year under a fifth scenario. As an example, based on these performance results, the analytic engine 132 can calculate a value of 0.4 for a performance metric, indicating that the rule was met in only 40% of the tested scenarios. As another example, based on these performance results, the analytic engine 132 can determine a value of 0 for a performance metric, indicating that the rule was not met (for example, based on a number of treatments averaged over the different scenarios, 3800 treatments, being less than the minimum 4000 treatments; based on the rule not being met in a single scenario; based on the rule not being met in a threshold number or percent of scenarios; etc.). As another example, based on these performance results, the analytic engine 132 can determine a value of 200 for a performance metric, indicating that the difference between the minimum number of treatments per year and the average estimated number of treatments per year is −200 treatments. As another example, based on these performance results, the analytic engine 132 can determine a vector of values of [0, 0.05] for a performance metric, indicating (i) through the first value that the rule was not met and (ii) through the second value that the average estimated number of treatments per year was 5% less than the minimum number of treatments.

In some implementations, in simulating the model 130a, the analytic engine 132 generates one or more distributions of performance results. The analytic engine 132 can then calculate the performance metrics for the model 130a using the one or more distributions. As an example, the analytic engine 132 can generate a distribution of performance results for a particular pharmaceutical therapy distribution constraint or parameter such as the anticipated number of treatments over time for multiple, different scenarios. The distribution results can indicate, for example, that more than 4300 treatments are anticipated at the end of the first year of the agreement (for example, at Jan. 1, 2025) under a first scenario, that between 3800 and 3900 treatments are anticipated at the end of the first year of the agreement under five other scenarios, that between 3600 and 3800 treatments are anticipated at the end of the first year of the agreement under ten other scenarios, that between 3300-3400 treatments are anticipated at the end of the first year of the agreement under four other scenarios, and that less than 3200 treatments are anticipated at the end of the first year of the agreement under a final scenario. The analytic engine 132 may use a portion of the distribution to calculate the performance metrics for this pharmaceutical therapy distribution constraint or parameter of the model 130a. For example, the analytic engine 132 may evaluate only those results that are within a standard deviation of the distribution, resulting in at least the results from the first scenario and the final scenario being removed from the evaluation. The analytic engine 132 can then use the subset of performance results to calculate one or more performance metrics, such as a value of 0 for a performance metric indicating that 100% of the performance results in the subset failed to meet the pharmaceutical therapy distribution constraint 114.

In some implementations, the third-party computing device 108 makes available at least a portion of the performance results to one or more of the parties of the distribution model 130. For example, after simulating the distribution model 130a for five different scenarios and obtaining five sets of performance results, the third-party computing device 108 can transmit the five sets of performance results to the distributor computing system 102 and the supplier computing system 104 or otherwise make those results accessible. The results may be displayed, at least in part, using one or more graphs corresponding to different pharmaceutical therapy distribution constraints, different scenarios, or the like. As an example, the performance results can indicate which, the number of, or percent of supplier-defined pharmaceutical therapy distribution constraints that were met or not met. Similarly, the performance results can, for example, indicate which, the number of, or percent of distributor-defined pharmaceutical therapy distribution constraints that were met or not met.

In some implementations, the third-party computing device 108 makes available one or more performance metrics determined for the distribution model 130. For example, after evaluating five sets of performance results from simulating the model 130a under five different scenarios, the third-party computing device 108 can calculate a set of performance metrics summarizing the performance of the model 130a. The third-party computing device 108 can transmit these performance metrics to the distributor computing system 102 and/or the supplier computing system 104, with or without the simulation results. Alternatively, the third-party computing device 108 can make the performance metrics accessible to users of the distributor computing system 102 and/or the supplier computing system 104. For example, the third-party computing system 108 can present a user interface showing the calculated performance metrics alongside a plot of the performance results. The interface may allow a user to select and view different performance areas for the model 130a, such as areas of insufficient performance, areas corresponding to a particular pharmaceutical therapy distribution constraint, etc. In response to such a selection, the interface can be updated by the third-party computing system 108 to display performance information related to the selection (for example, a graphed distribution of simulated values over time for a first pharmaceutical therapy distribution constraint) and/or corresponding performance metrics (for example, indications of whether any of the values in the distribution of simulated values failed to meet any pharmaceutical therapy distribution constraints such as the rules described above).

The third-party computing device 108 uses the performance results from the analytic engine 132 to perform one or more model update(s) (134) to produce an updated pharmaceutical distribution model 130b. The decision of how to update the model 130a may be made by the analytic engine 132 of the third-party computing device 108. The analytic engine 132 may perform the model update(s) (134), for example, using one or more programs. As an example, the analytic engine 132 analyzes the performance results from testing (for example, simulating) the model 130a under five different scenarios. The performance results of the simulation can be compared to a set of pharmaceutical therapy distribution constraints for the model 130a to calculate a set of performance metrics for the model 130a. Using the performance results and/or the performance metrics for the model 130a, the third-party computing system 108 (for example, the analytic engine 132 of the third-party computing system 108) updates the model 130. Additionally or alternatively, the third-party computing system 108 (for example, the analytic engine 132 of the third-party computing system 108) generates a set of recommended updates that are then made accessible to the distributor computing system 102 and/or the supplier computing system 104 for approval or disapproval. The updates generated by the third-party computing system 108 can include new definitions for one or more pharmaceutical therapy distribution constraints of the model 130, adjustments to value(s) assigned to one or more parameters of the model 130, or a combination thereof to improve or optimize the model.

In some implementations, the updates generated by the third-party computing system 108 are based on the performance metrics calculated for the model 130a. For example, if a performance metric indicating the difference between the anticipated number of treatments and the minimum number of treatments is −200, the analytic engine 132 can generate an update to the model 130 to reduce the minimum number of treatments by at least 200 treatments per year.

In some implementations, improving or optimizing the model 130a includes adjusting the model 130a to obtain improved performance metrics. For example, if the performance metrics indicate that the model 130a failed to meet five different pharmaceutical therapy distribution constraints, the analytic model 108 may update the model 130 to redefine one or more pharmaceutical therapy distribution constraints, adjust values assigned to parameters of one or more pharmaceutical therapy distribution constraints, or both in a manner that is predicted to produce new performance results for the model 130 that will meet the five different pharmaceutical therapy distribution constraints. As another example, if the performance metrics indicate that the model 130a failed to meet five different pharmaceutical therapy distribution constraints, the analytic model 108 may update the model 130 to redefine one or more pharmaceutical therapy distribution constraints, adjust values assigned to parameters of one or more pharmaceutical therapy distribution constraints, or both and retest the model 130 with the updates. This process can be repeated by the third-party computing system 108 one or more times, for example, until the observed performance results meet the five different pharmaceutical therapy distribution constraints. One or more of the updates can be intelligently made by the third-party computing system 108 based on, for example, performance metrics calculated using the performance results of the model 130a.

Additionally or alternatively, one or more of the updates can be made randomly or pseudo-randomly by the third-party computing system 108.

As an example, the analytic engine 132 can determine a value of 0 for each of five different variants of a first performance metric, each corresponding to a particular scenario tested. The value of 0 for the first performance metric indicates, for example, that the minimum treatment supply is greater than the anticipated number of treatments for each of the scenarios. In response, the analytic engine 132 can update the model 130 to reduce the minimum treatment supply from 4000 treatments per year to 3700 treatments per year and retest the model 130 using the same five scenarios, resulting in, for example, the generation of the model 130b. The performance results can improve the performance metrics such that the analytic engine 132 now determines a value of 1 for four out of the five different variants. The analytic engine 132 can apply a threshold percent of 0.8 to determine that the model 130b has met the minimum required performance for this metric, for example, the model 130b provides sufficient performance in the minimum treatment supply being less than or equal to the estimated number of treatments in at least 80% of the scenarios tested.

In some implementations, improving or optimizing the model 130a includes adjusting the model 130a to reduce or eliminate the number of pharmaceutical therapy distribution constraint violations. The pharmaceutical therapy distribution constraint (for example, performance rule) violations can include all violations of all pharmaceutical therapy distribution constraints or a subset of pharmaceutical therapy distribution constraints (for example, that the third-party computing system 108 has determined are more relevant, that a supplier has indicated that they are not willing to have adjusted or have different values assigned to, that a distributor has indicated that they are not willing to have adjusted or have different values assigned to, etc.).

In some implementations, improving or optimizing the model 130a includes performing one or more additional simulations of the model 130 using determined updates. For example, the analytic engine 132 may randomly generate new values for one or more parameters (for example, parameters open to negotiation, parameters of pharmaceutical therapy distribution constraints associated with poor performance from the initial performance results, etc.) and retest the model 130 using the updated values by performing one or more simulations with the updated model 130. The analytic engine 132 may then analyze the new performance results. If the performance results indicate an improvement in one or more performance areas, the analytic engine 132 may identify values associated with those one or more performance areas and then, of those, the values that were updated. The identified subset of values may be locked or temporarily locked for the model 130. For example, if a performance metric associated with a pharmaceutical therapy distribution constraint for the anticipated number of treatments required per year indicates that the anticipated number of treatments is greater than minimum number of treatments (for example, for all scenarios tested, or for more than a threshold number or percent of scenarios tested) and if the updated values included one or more values assigned to one or more parameters of the pharmaceutical therapy distribution constraint, then the analytic engine 132 can assign keep those values assigned to the parameters of the model 130. Similarly, if the performance results indicate a reduction in performance in one or more performance areas or no increase in performance in one or more performance areas, the analytic engine 132 may identify values associated with those one or more performance areas and then, of those, the values that were updated. The analytic engine 132 can unlock the values of those one or more parameters such that new values can be randomly assigned to the one or more parameters, or, alternatively, the previous used values are re-assigned to the one or more parameters (for example, values assigned to the parameters of the model 130a). These techniques can be repeated multiple times to improve and/or optimize the model 130. The model 130b can be the result of applying these techniques one or more times.

In some implementations, improving or optimizing the model 130a includes reducing or minimizing the distance between one or more performance results and one or more desired performance results. As an example, a desired number of anticipated treatments per year can be set equal to 1.1 multiplied by the minimum number of treatments per year to give the distributor a 10% buffer to account for less likely scenarios without reducing minimum treatment supply below levels that would be acceptable to the supplier This pharmaceutical therapy distribution constraint may be negotiated between and/or agreed upon by the distributor and supplier. The distance reduced or minimized can be a distance between two values, a distance between a value and a vector of values, a distance between two vectors (for example, a Euclidean distance), a distance between two matrices, etc. As an example, if the average calculated value of the anticipated number of treatments is equal to 0.95 multiplied by the minimum number of treatments, the distance can be calculated as 600 when the minimum treatment supply is equal to 4000 treatments per year. In response, the analytic engine 132 can improve and/or optimize the model 130 by reducing the minimum treatment supply to 3400. As another example, the anticipated number of treatments can be represented by a vector with each value corresponding to a different value, such as [4500, 4000, 3700, 3500, 3300]. The analytic engine 132 can then calculate a distance between the vector and the desired value of 4400 when the minimum number of treatments is set to a value of 4000.

In some implementations, the third-party computing system 108 prefers improving the model 130 over optimizing the model 130. For example, the third-party computing system 108 may stop the process of updating the model 130 once the performance metrics indicate sufficient performance of the model to reduce use of computing resources and, therefore, to improve computing efficiency. The model 130b can represent an improved pharmaceutical distribution model.

In some implementations, the third-party computing system 108 prefers to optimize the model 130 over improving the model 130. For example, the third-party computing system 108 may optimize the model 130 by repeatedly simulating the model 130, analyzing the performance results of the model 130, and updating (for example, generating a test model) the model 130 until the model 130 is optimized (for example, each of the performance metrics calculated from the performance results matches a desired performance metric). The model 130b can represent an optimized pharmaceutical distribution model.

In some implementations, a set number of rounds of negotiation are set for the generation of a particular model. For example, the information 110a and/or the information 110b can include an indication of the preferred number of rounds of negotiation for the distributor and/or supplier respectively. As an example, if the information 110a includes an indication that the distributor prefers three rounds of negotiation and the information 110b also include an indication the supplier prefers three rounds of negotiation, the third-party computing system 108 can set the number of rounds of negotiation to a maximum of three (for example, parties may agree to a model before the three rounds end). As another example, the third-party computing system 108 may include a default number of rounds (for example, two rounds, three rounds, four rounds, etc.) that is optionally adjustable by a party of the agreement (for example, party initializing the agreement).

As an example, if the number of rounds of negotiation for the model 130 is set to three, then the third-party computing system 108 can make up to three different versions of the model 130 available to the distributor computing system 102 and the supplier computing system 104 based on feedback received from the distributor computing system 102 and/or the supplier computing system 104. In more detail, during a first round where the generation of the model 130 is initialized by the supplier computing system 104, the supplier computing system 104 defines a number of pharmaceutical therapy distribution constraints and/or sets values for a number of parameters for the model 130. The third-party computing system 108 can then make the initial version of the model 130, the initial pharmaceutical therapy distribution constraints for the model 130, and/or the initial values for the parameters of the model 130 are made available to the distributor computing system 102. The distributor computing system 102 may then provide input (for example, feedback) to the third-party computing system 108 to approve all or part of the model 130, redefine one or more pharmaceutical therapy distribution constraints of the model 130, reassign one or more new values to one or more parameters of the model 130. The third-party computing system 108 can then update the model 130 based on the feedback received, indicating the start of the second round. The third-party computing system 108 can then make the updated model 130 available to the supplier computing system 104 and request feedback.

In the process of generating the model 130, the third-party computing system 108 may receive feedback from the distributor and/or the supplier. For example, during a round of negotiation, the third-party computing system 108 may receive feedback in the form of adjustments to one or more pharmaceutical therapy distribution constraints of the model 130, adjustments to one or more values assigned to one or more parameters of the model 130, approval of one or more pharmaceutical therapy distribution constraints or parameter values, denial of one or more pharmaceutical therapy distribution constraints or parameter values, approval of the current version of the model 130, denial of the current version of the model 130, or a combination thereof.

The model 130a shown in FIG. 1A can represent the initial version of the model parameter values set by the party initializing the model 130). Alternatively, the model 130a can represent a version of the model 130 after one or more rounds of negotiation.

The model 130b shown in FIG. 1B can represent a version of the model 130 after one or more rounds of negotiation. As an example, the model 130b can represent the final version of the model 130 after the end of the last round of negotiation or after approval of the current version of the model 130 is received from each of the parties (for example, the supplier and the distributor).

In some implementations, the model 130 represents an outcomes-based agreement (for example, OBA). For example, the model 130 can include one or more pharmaceutical therapy distribution constraints that provide for the distributor paying a first amount upfront and paying a second amount layer if patients provided the treatment have positive outcomes, where the first amount is significantly less than the second amount. Additionally or alternatively, the model 130 can include one or more pharmaceutical therapy distribution constraints that provide for the distributor paying an amount upfront and receiving a rebate for at least part of the amount if patients provided the treatment have negative outcomes.

As another example, the model 130a includes one or more pharmaceutical therapy distribution constraints such that if the number of used treatments is less than a minimum treatment amount to be supplied, the minimum treatment amount to be supplied is reduced to 10% below the average number of treatments used over the past year.

In some implementations, the model 130 includes one or more pharmaceutical therapy distribution constraints that account for patient or treatment volume. For example, the model 130 can include the following pharmaceutical therapy distribution constraint(s): if the total number of treatments used is less than 10,000, the supplier charges a first price per treatment for each of the treatments supplied; if the total number of treatments is greater than or equal to 10,000 but less than 20,000, the supplier charges a second price per treatment for each of the treatments supplied, where the second price is less than the first price; and if the total number of treatments is greater than or equal to 20,000, the supplier charges a third price per treatment for each of the treatments supplied, where the third price is less than the first and second prices.

As another example, the model 130 can include a tiered system for pricing treatments based on the number of patients such that the supplier charges a first amount for a first X number of patients (for example, patients in a first tier) then charges a different, lower amount for any patients beyond the X number of patients (for example, patients in a second tier). Although this example describes a two-tier system, any number of pricing tiers is possible.

In some implementations, the model 130 includes one or more pharmaceutical therapy distribution constraints that provide a revenue volume discount. For example, if the distributor spends at least X amount in the first year of the distribution agreement represented by the model 130, then the supplier reduces the cost per treatment for any further purchases in the first year of the distribution agreement or for the remaining duration of the distribution agreement.

In some implementations, the model 130 includes one or more pharmaceutical therapy distribution constraints that cap the number of treatments or the number of patients. For example, the model 130 can include a pharmaceutical therapy distribution constraint that limits the number of treatments supplied to a maximum of 6000 units per year due to manufacturing pharmaceutical therapy distribution constraints of the supplier.

As another example, the model 130 can include a pharmaceutical therapy distribution constraint that limits the amount the distributor pays to $100 M per year (or per contract) for the entire population of treated patients. After the distributor pays this amount in the current contract year (or while the contract is in effect), the supplier may be required to provide a rebate, free units of the treatment, and/or reduced-cost units of the treatment.

As another example, the model 130 can include one or more pharmaceutical therapy distribution constraints that limit the amount the distributor pays to $100 M per year (or per contract) for the entire population of treated patients and includes a maximum rebate of $30 M per year (or per contract). In more detail, if the distributor pays $100 M in the first year of the contract, then supplier provides them a rebate of $30 M in purchases of the treatment. However, if the distributor exceeds $130 M in purchases, then the rebate ends and the distributor will be required to resume full-priced purchases of the treatment.

In some implementations, the model 130 includes one or more pharmaceutical therapy distribution constraints to address individual patients. For example, one or more pharmaceutical therapy distribution constraints of the model 130 can limit the amount that the distributor must pay treating a single patient with the treatment to $100,000 per year or per contract. Should the expense reach this amount, the supplier may need to provide a rebate, free units of the treatment, and/or reduced-cost units of the treatment for the particular patient for the current year or for the remaining duration of the contract.

As another example, one or more pharmaceutical therapy distribution constraints of the model 130 can limit the time that the distributor must pay for treating a single patient to the first 10 months of treatment, after which the supplier may need to provide a rebate, free units of the treatment, and/or reduced-cost units of the treatment for the particular patient.

In some implementations, the model 130 includes one or more pharmaceutical therapy distribution constraints that set an unlimited number of patients or supply of treatments. For example, the model 130 can include one or more pharmaceutical therapy distribution constraints that require the distributor to pay a large set amount mor each year of the contract duration. However, in return, the distributor receives from the supplier as much supply of the treatment as needed.

In some implementations, the model 130 includes one or more pharmaceutical therapy distribution constraints that specify conditions for when treatments are supplied free of charge. For example, a pharmaceutical therapy distribution constraint of the model 130 can specify that the first three treatments to any patient are free of charge to the distributor. If the distributor already paid for the treatments used to treat the patients, the supplier can be required to provide the next three units of the treatment for free.

As another example, a pharmaceutical therapy distribution constraint of the model 130 can specify that for every 100 units of the treatment (for example, packs of the treatment) purchased by the distributor, the supplier provides 20 units of the treatment free of charge.

In some implementations, the model 130 includes one or more pharmaceutical therapy distribution constraints regarding the authorization of the treatment in a particular region. For example, the model 130 can include one or more pharmaceutical therapy distribution constraints that specify that the distributor can treat patients with no upfront cost of the treatment prior to full regulatory approval (for example, FDA approval) under a special scheme in a particular region (for example, U.S.). Then after authorization and price is determined, the distributor is required to pay the supplier for all of the prior treatments at a price that is less than the determined price (for example, 75% of the determined price). If, however, the treatment is not authorized, the distributor is not required to pay for the treatments acquired from the supplier.

FIGS. 2A-2B are exemplary graphical user interfaces depicting environments for distributing a pharmaceutical therapy.

FIG. 2A is an exemplary graphical user interface 200 depicting an environment for distributing a pharmaceutical therapy. As an example, the graphical user interface 200 is an interface for initializing a pharmaceutical distribution model (for example, the model 130 shown in FIGS. 1A-1B as described above) for the distribution of 100 mg/4 ml vial of Keytruda.

In some implementations, the interface 200 is presented by the third-party computing system 108 shown in FIGS. 1A-1B and described above. For example, the interface 200 is presented as part of a sandbox environment hosted by the third-party computing system 108.

The interface 200 includes a first interface area 202 (for example, first section) for assumptions applied to the model. These assumptions include, for example, an agreement (for example, contract) start date, an agreement duration (for example, contract length), an estimated number of patients for the first year of the agreement, etc. The first interface area 202 may include an interface element (for example, a link, an interactive button, etc.) for patient distribution. In response to a user selecting this interface element, a new interface or a pop-up window containing patient distribution information may be presented to the user such as the interface 250 shown in FIG. 2B. The first interface area 202 may include an interface element (for example, link, interactive button, etc.) to display advanced assumption. In response to a user selecting this interface element, the interface area 202 can be extended to show additional fields and/or options where a user can enter or select additional assumptions for the model. These additional assumption can include, for example, a number of patients for one or more other years, an estimated rate or increase in new patients per year, an estimated rate or decrease in existing patients per year, etc.

The interface 200 includes a second interface area 210 (for example, second section) for the structure of the model. The structure (for example, agreement structure) of the model can include financial details, such as pricing, a populating level capacity, an individual level capacity, a population level free of charge (FOC), an individual level free of charge (FOC), etc. The structure of the model can additionally or alternatively include performance details, such as therapy discontinuation. Each of the structure elements of the model can be, for example, presented with an interactive interface element such as a check box. A user can interact with the interactive interface element to, for example, select or unselect a particular structure element of the model. In response to selecting an interactive interface element for a particular structure element, the interface can be updated (for example, by the third-party computing system 108) in real-time to provide an interface area where a user can view details of the particular structure element and/or can adjust the details of the particular structure element. The interface 200 includes a third interface area 220 (for example, third section) for pricing details. For example, the third interface area 220 is presented in response to a user selecting the pricing checkbox associated with the “Pricing” structure element for the model. The pricing details include, for example, one or more of the following: a pricing strategy drop-down menu (for example, with a per patient price option, with fixed price option, with a per therapy unit/pack/cycle option, etc.), a price basis drop-down menu (for example, with a cycle basis option), an interface element to select an option to use tiers, a tier type drop-down menu (for example, with a patient option, with a therapy unit/pack/cycle option, etc.), a pricing type interface element (for example, with a tiered pricing type option, a volume pricing option, etc.), one or more fields corresponding to two or more tiers if tiers are used (for example, with a max patients field, a net price per cycle field, and a net discount field), or an interactive interface elements that permits a user to increase the number of tiers or reduce the number of tiers.

The pricing details can also include list price analysis section with one or more additional pricing details specifically related to the therapy to be distributed. For example, the pricing details in the list price analysis section can include one or more of the following details related to the 100 mg/4 ml vial Keytruda therapy: a field for the per patient pack utilization in packs per year, a field for the list price per pack, a field for the annual list price patient, a field for the cycle list price per patient, or a field for the weekly list price per patient. The interface elements and/or values in the list price analysis section can change in response to a change in therapy. The interface elements in the list price analysis section can change in response to a change to the pricing strategy and/or price basis.

One or more of the pricing details may be updated in real-time based on information entered. For example, for a first tier of 1-1000 patients, if the cycle list price per patient is 67,039 is populated (for example, automatically based on a look-up table with information about the 100 mg/4 ml dosage of Keytruda) and a user enters 25.416 into the net discount field (for example, when set to percent instead of cost), then the net price per cycle field is automatically populated with a value of 50,000 based on multiplying the 67,039 cost by 0.7458 to account for the discount.

One or more pricing details may be filled automatically (for example, by the third-party computing system 108). For example, the third-party computing system 108 can use one or more algorithms or one or more lookup tables to fill in pricing details (or other details of the model presented in the interface 200). For example, based on the selection of the 100 mg/4 ml vial of Keytruda and/or a condition (for example, diagnosis) to treat with the therapy, the third-party computing system 108 can use a lookup table to automatically fill one or more of the following values: the value of 34.76 for the per patient pack utilization in packs per year, the value of 33,519.50 for the list price per pack, the value of 1,165,201.67 for the annual list price per patient (or, alternatively, is automatically calculated by multiplying the value in the per patient pack utilization field by the value in the list price per pack field), or the value of 67,039 for the cycle list price per patient, the value of 22,346.33 for the weekly list price per patient (or, alternatively, is automatically calculated by dividing the value in the annual list price per patient field by 52).

The interface 200 includes a fourth interface area 230 (for example, a fourth section) for analytics for the model. The details in the fourth interface area 230 can include analytic details that are, for example, automatically filled in (for example, by the third-party computing system 108, an analytic tool of the third-party computing system 108) based on other details of the model (for example, the assumptions provided in the first interface area 202, the pricing details provided in the third-interface area 220, etc.). The details in the fourth interface area 230 can include, for example, values predicted (for example, by the third-party computing system 108, an analytic tool of the third-party computing system 108) based on other details of the model (for example, the assumptions provided in the first interface area 202, the pricing details provided in the third-interface area 220, etc.).

The details in the fourth interface area 230 can include details for population level analytics. As an example, the population level analytics details can include one or more of the following: an average annual net revenue, a total list revenue, a total net revenue, an average net revenue per patient, a total rebate percent, an average pack price, a total list revenue, a total net revenue, a total net revenue net present value (NPV), a total number of patients for the duration of the model (for example, contract length), a total price rebate, a total capacity rebate, or a total rebate.

The details in the fourth interface area 230 can include details for individual patient level analytics. As an example, the individual patient level analytics details can include one or more of the following: an average list revenue per patient, an average net revenue per patient, an average net revenue NPV per patient, an average rebate per patient, or an average treatment length (for example, in months, years, weeks, days, etc.).

The details in the fourth interface area 230 can include details for pack level analytics. As an example, the pack level analytics details can include one or more of the following: average net price per pack, a total pack utilization, or an average pack utilization per patient.

In some implementations, the fourth interface area 230 includes one or more graphical elements to display analytic details and/or compare analytic details. In more detail, the fourth interface area 230 can include a bar graph showing the net revenue and the total rebate for each year that the model is in effect.

In some implementations, one or more notable details in the fourth interface area 230 are highlighted or are added to a spotlight section in the fourth interface area 230. For example, an average annual net revenue, an average net revenue per patient, a total rebate percent, and an average pack price details can be added to a spotlight section at the top of fourth interface area 230 to highlight these details (for example, determined by the third-party computing system 108 to typically be the most notable analytic details).

In some implementations, a user can adjust values for one or more details provided in the fourth interface area 230. As an example, a user may be able to select the average annual net revenue field and adjust the value. In response, the third-party computing system 108 may update the interface 200 by automatically changing the assumptions in the first interface area 202 and/or the pricing details in the third interface area 220 to accomplish the average annual net revenue field value desired by the user. Additionally or alternatively, the third-party computing system 108 may present (for example, in a pop-up window) one or more recommendations to adjust the assumptions in the first interface area 202 and/or the pricing details in the third interface area 220 to accomplish the average annual net revenue field value desired by the user.

In some implementations, the fourth interface area 230 includes an interface element 232 for an annual breakdown of the analytic details. As an example, in response to a user selecting the interface element 232, the third-party computing system 108 can present the user with a new interface in the form of a new window or a pop-up window that shows analytic details broken down by year (for example, each year that the model is in effect, each year of the contract duration, etc.).

FIG. 2B is an exemplary graphical user interface 250 depicting an environment or part of an environment for distributing a pharmaceutical therapy. As an example, the interface 250 is an interface displaying patient distribution assumptions. The interface 250 may be presented to a user (for example, by the third-party computing system 108) in response to a user selecting an interface option for patient distribution assumptions in the interface 200 shown in FIG. 2A and described above.

As an example, the patient distribution assumptions displayed in the interface 250 include one or more of the following: a contract start date, a contract length, a cashflow calculation horizon, an estimated number of patients in the first year the model is in effect (for example, first year of the contract), or a patient distribution method. A user can interact with corresponding fields or other interface elements (for example, drop-down menus) for these assumptions to add values or adjust values.

The interface 250 may include assumptions for different time periods that the model is in effect. The time periods can include, for example, a one year period for each year model is in effect. As an example, the interface 250 can include one or more of the following: a new patient growth rate 264 with a field and/or value for each of the three years that the example model is effect, a new patient dropout rate 266 with a field and/or value for each of the three years that the example model is effect,

In some implementations, one or more assumptions displayed in the interface 250 are presented with a default or predetermined value. As an example, the third-party computing system 108 can set values for the assumptions shown in the interface 250 based on assumption values obtained from a previously generated pharmaceutical distribution model. As another example, the third-party computing system 108 can set values for the assumptions shown in the interface 250 based on default values (for example, for the particular condition being treated, the particular therapy, the particular supplier, the particular distributor, etc.).

In some implementations, one or more details displayed in the interface 250 are populated with details obtained from the interface 200 shown in FIG. 2A and described above. For example, the contract start date field, the contract length field, and the estimated number of patients in the first year field shown in the interface 250 are populated with values from corresponding fields shown in the first interface area 202 of the interface 200 shown in FIG. 2A.

In some implementations, the interface 250 includes one or more graphical elements based on the patient distribution assumptions. As an example, the interface 250 may include the graph 262 showing the monthly patient distribution with anticipated new patients, dropped patients, and treated patients over the duration that the model is in effect.

In some implementations, the interface 250 includes an interface element to close the interface 250. For example, a user can interact with the interface element 274 to close the interface 250 to go back to the interface 200 shown in FIG. 2A.

In some implementations, adjustments made through the interface 250 are carried through to the interface 200 shown in FIG. 2A. For example, if a user changes the contract length field 274 from three years to four years, the third-party computing system 108 can update the interface 200 shown in FIG. 2A to update the corresponding contract length field shown in the FIG. 2A to reflect the four year value.

FIGS. 3A-3B are exemplary graphical user interfaces depicting environments for distributing a pharmaceutical therapy.

FIG. 3A is an exemplary graphical user interface 300 depicting an environment for distributing a pharmaceutical therapy. As an example, the graphical user interface 300 is an interface presented to a supplier (for example, MSD) during a first round of negotiating a pharmaceutical distribution model (for example, the model 130 shown in FIGS. 1A-1B as described above) for the distribution of a therapy (for example, 100 mg/4 ml vial of Keytruda) for the treatment of a displayed condition 301 (for example, advanced melanoma). The user interface 300 can display a date range 302 for the duration that the model is in effect (for example, Jan. 1, 2023 through Dec. 31, 2025), a distributor 303 for the therapy (for example, Sweden NT), a currency 304 for the model (for example, Swedish Krona), and a maximum number of rounds 305 for negotiating the pharmaceutical therapy distribution constraints and/or parameters of the model (for example, a maximum of three rounds). The user interface 300 can also display a phase indicator 306 that indicates the phase (for example, stage) in the process of generating or finalizing a pharmaceutical distribution model. As shown, the indicator 306 indicates that the current phase is “Round 1” and that the response has not yet been submitted to the distributor.

In some implementations, the interface 300 is presented by the third-party computing system 108 shown in FIGS. 1A-1B and described above. For example, the interface 300 is presented as part of a sandbox environment hosted by the third-party computing system 108.

In some implementations, the interface 300 is based on selections made in the interface 200 shown in FIG. 2A described above. For example, after a supplier or distributor is satisfied with the selections made in the initialization interface 200 shown in FIG. 2A, the supplier or distributor can interact with an interface element to select the initial model specified through the initialization interface 200 as the basis for negotiation. In response, the third-party computing system 108 may identify a counterparty and transmit a notification to the counterparty. The notification can include a link that would bring the counterparty to the interface 300 (for example, in the case when a distributor initialized the model).

The interface 300 includes a first interface area 310 (for example, first section) for the model structure and structure details. The first interface area 310 includes a section 312 listing one or more structure components for the model. For example, the section 312 includes one or more of the following: a pricing component 313 (for example, for setting the per patient price of a treatment cycle), an overall populating capacity component 314, an individual patient capacity component 315, an overall population free of charge (FOC) component 316, an individual patient FOC component 317, or a performance component 318.

The interface 300 includes an interface area 320 (for example, pricing section) corresponding to the pricing component 313 of the model. As an example, the interface area 320 is presented in response to a user selecting the pricing component 313. The interface area 320 can display pricing details including, for example, one or more of the following: an indicator 322 indicating whether pricing is a price input per cycle or a price input per pack of the therapy, pricing tier information (for example, max patients, price per cycle, discount in percent or amount, etc.), or pricing analytics information.

In some implementations, the interface area 320 displays pricing tier information when the model includes pricing tiers. The information can include fields for max patients for the tier, price per cycle for the tier, and a discount for the tier. As an example, the interface area 320 can display pricing information 323 for a first tier for the first 1000 patients, pricing information 324 for a second tier for the next 2000 patients, and pricing information 325 for any additional patients.

In some implementations, the interface area 320 displays pricing analytics. As an example, the interface area 320 displays a chart 326 for the specified therapy. The chart can include information about the therapy, such as a drug or product name, a dosage or primary pack information for the therapy, a list price for the therapy units (for example, primary pack), a number of units required (for example, packs required per cycle), and a cycle list cost (for example, the list price multiplied by the number of therapy units).

A user can interact with one or more of the interface elements in the interface area 320 to adjust values of the pricing details. For example, a user can interact with the indicator 322 to change pricing from price input per cycle to price input per pack.

The interface area 310 can include an interface element 327 for saving and recalculating analytics. For example, after making changes to one or more of the pricing details in the interface area 320, a user can select the interface element 327 to save the model with the new values and to update the interface 300 to reflect new analytics calculated using the new values. Alternatively, the analytics may be recalculated in real-time as pricing details are adjusted.

The interface area 310 can include an interface element 328 to review and submit the model to a counterparty. For example, after the supplier (for example, user of the supplier computing system 104 shown in FIGS. 1A-1B, MSD, user accessing the third-party computing system 108 through an MSD device, etc.) is satisfied with the displayed version of the model, the supplier can interact with the interface element 328 to review the model and submit the model to the selected distributor (for example, Sweden NT). In response to an interaction with the interface element 328, the third-party computing system 108 can, for example, generate a version of the model (for example, read only version) and provide a copy of the model to the supplier (for example, display a read-only version of the model, provide a link to download a copy of the model, etc.). In response to an interaction with the interface element 328 or after an interaction with the interface element 328, the third-party computing system 108 can, for example, generate a notification and transmit a notification to a device of the distributor (for example, Sweden NT). The notification can include a link to the current version of the model.

The interface 300 includes an interface area 330 for a revenue forecast of the model. The interface area 330 can display one or more assumptions for the model, such as an assumption 331 for a length that the model is to be in effect (for example, contract length of three years), an assumption 332 for an estimated number of patients per year (for example, 10,000 patients), and/or an assumption 333 for an annual patient growth rate (for example, in percent). The interface area 330 can display therapy information 334, such as a number of packs of the therapy required (for example, per year, per the duration of the model, etc.) and/or a pack list price for the therapy. The interface area 330 can display revenue forecast information, such as an estimated net revenue, an estimated gross revenue, an estimated rebate, an average price discount, an average net patient price, an average net pack price, and/or an estimated pack utilization.

In some implementations, the interface area 330 includes an annual breakdown section 336. The annual breakdown section 336 can include forecast information 338 of the model broken down by year (for example, each year that the model is to be in effect). The forecast information 338 can include a forecast 340 for the estimated number of patients at the end of each of the years, a forecast 342 of the net revenue at the end of each of the years, and/or a forecast 344 of the gross revenue at the end of each of the years. The annual breakdown section 336 may also include one or more graphical elements that display a subset of the forecast information 338 in a graph format, such as the bar graph 337 that provides a visual representation of the net revenue from the forecast 342.

In some implementations, a user can interact with a notes interface element to add notes to the model. These notes may be viewable only by the current party (for example, supplier or distributor) but not the counterparty (for example, distributor or supplier). For example, a first user of the supplier computing system 104 shown in FIGS. 1A-1B can interact with the notes interface element to add notes to the model. These notes can later be viewed by the same user or a different user of the supplier computing system 104. In contrast, a user of the distributor computing system 102 shown in FIGS. 1A-1B is not able to view the notes.

FIG. 3B is an expanded view of the graphical user interface area 330 shown in FIG. 3A and described above. Many details about the contents of the interface area 330 are described above with respect to FIG. 3A. The expanded view of the graphical user interface area 330 includes additional details about the annual breakdown section 336. As an example, the forecast information 338 can also include a forecast 346 of an expected total rebate at the end of each of the years and/or a forecast 348 of an expected net patient price at the end of each of the years.

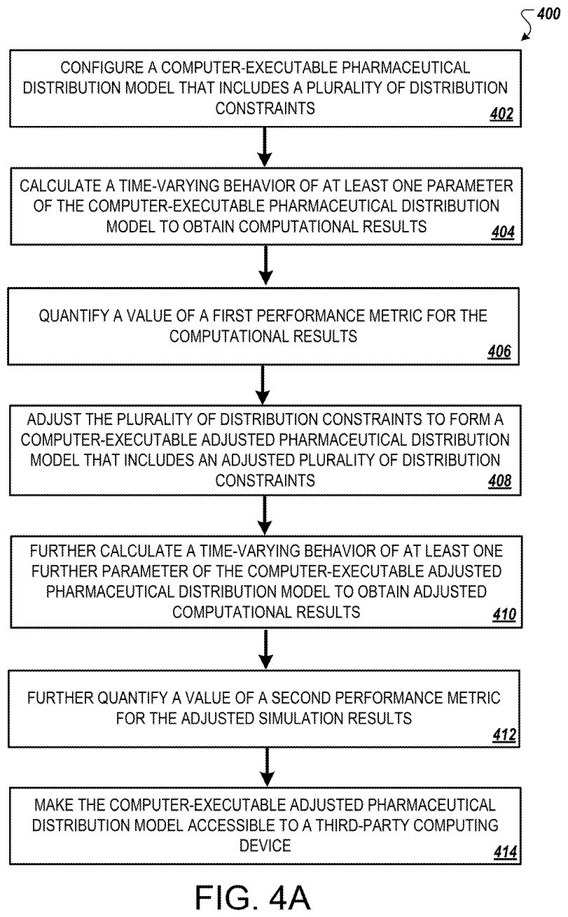

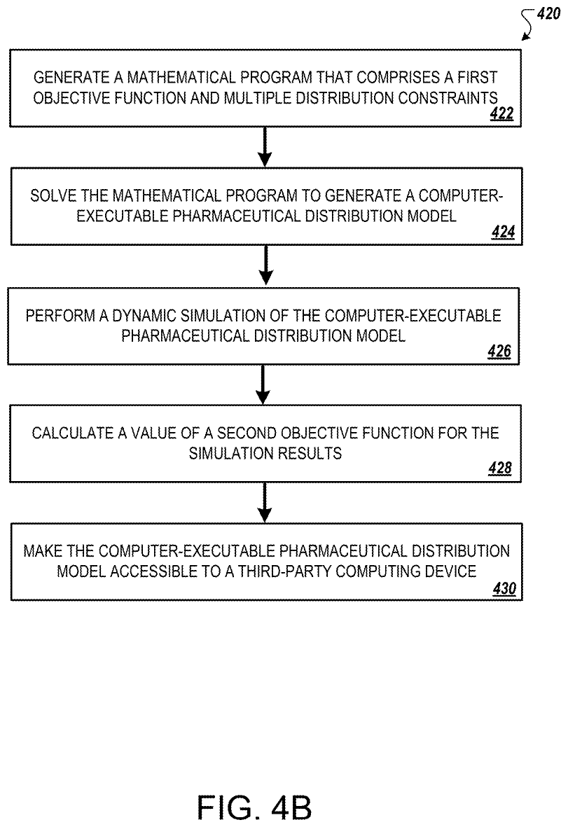

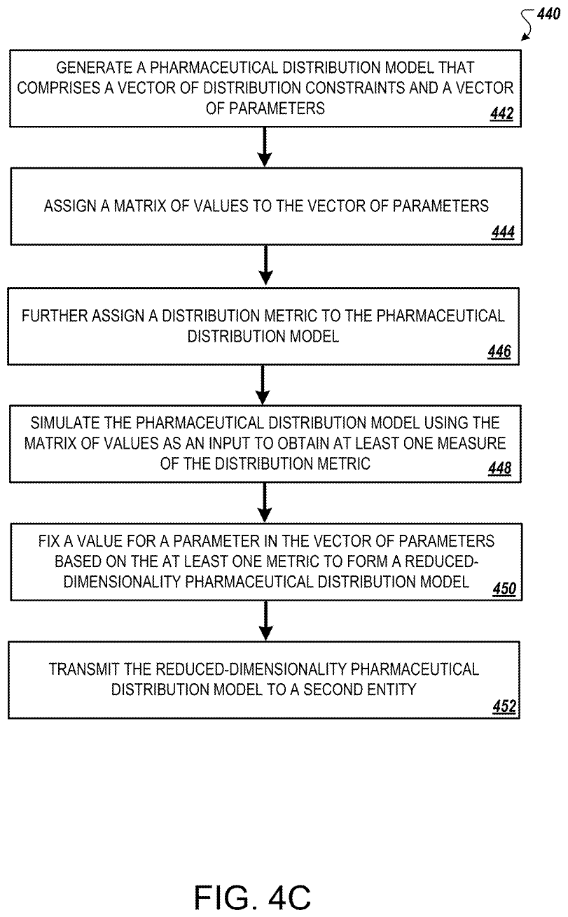

FIGS. 4A-4C are flow charts depicting example processes for distributing a pharmaceutical therapy.

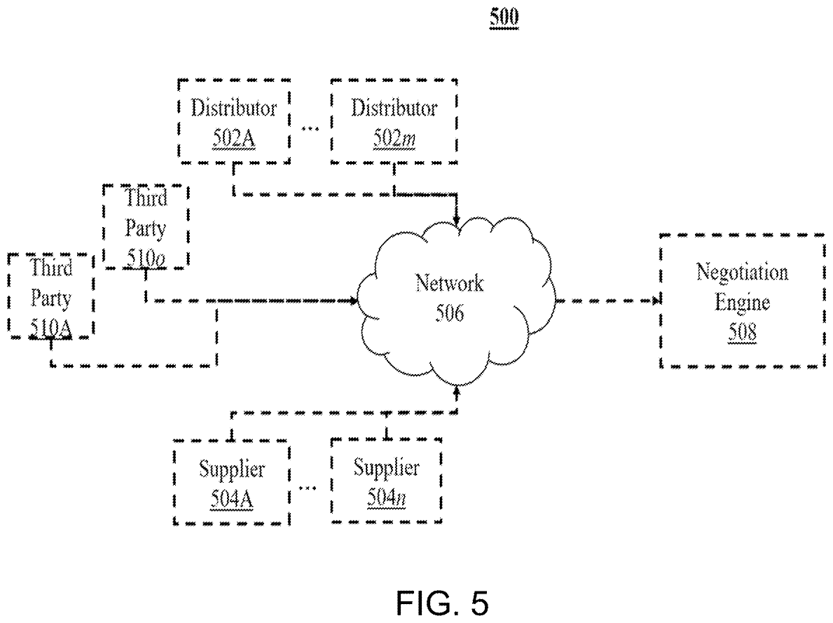

FIG. 4A is a flow chart depicting an example process 400 for distributing a pharmaceutical therapy. One or more of the steps, such as each of the steps, of the process 400 may be performed by one or more computers or computing systems. As an example, the process 400 is performed by the third-party computing system 108 (for example, third-party system with respect to the distributor computing system 102 and the supplier computing system 104) shown in FIGS. 1A-1B described above. As another example, the process 400 is performed by the negotiation engine 508 shown in FIG. 5 described below.

The process 400 includes configuring a computer-executable pharmaceutical distribution model that includes a plurality of distribution constraints (402). As an example, with respect to FIGS. 1A-1B, configuring a computer-executable pharmaceutical distribution model can include the third-party computing system 108 assigning initial values to all or a subset of parameters for the model 130 based on user inputs received at the third-party computing system 108, historical data obtained by or stored on the third-party computing system 108, calculations made by the third-party computing system 108 (for example, values calculated for one or more pharmaceutical therapy distribution constraints based on parameter values looked-up or provided by a user; randomized or pseudo-randomized values, etc.), or the like.

In some implementations, configuring the computer-executable pharmaceutical distribution model includes defining an initial structure for the model. For example, configuring the model can include defining one or more financial components for the model. These financial components can include one or more of the following: a pricing structure for the model, a population level capacity for the model, an individual level capacity for the model, a population level free of charge (FOC) for the model, or an individual level FOC. As another example, configuring the model can include defining one or more performance components for the model. These performance components can include one or more of the following: one or more conditions that trigger therapy discontinuation or termination of the model (for example, before the scheduled end date of the model, before an end of the contract date, etc.), one or more conditions that trigger a price reduction for the therapy (for example, therapy for a particular patient cost more than a maximum amount, therapy for a particular patient required more than a maximum number of doses/packs/units of the therapy, therapy for all patients in a year cost more than a maximum amount, therapy for all patients in a year required more than a maximum number of doses/packs/units of the therapy, etc.), or one or more conditions that trigger therapy extension or extension of the model (for example, extending a model for beyond the scheduled end date of the model, extending a contract duration for the model, etc.).

In some implementations, the computer-executable pharmaceutical distribution model is executable on a computing device or system that is accessible to both a supplier and a distributor. For example, with respect to FIGS. 1A-1B, the model 130 is executable on the third-party computing system 108 and is accessible to both users of the supplier computing system 104 and users of the distributor computing system 102.

In some implementations, the computer-executable pharmaceutical distribution model is executable on a computing device of a supplier. For example, with respect to FIGS. 1A-1B, the model 130 or a copy of the model 130 is executable on the supplier computing system 104.

In some implementations, the computer-executable pharmaceutical distribution model is executable on a computing device of a distributor. For example, with respect to FIGS. 1A-1B, the model 130 or a copy of the model 130 is executable on the distributor computing system 102.

In some implementations, the computer-executable pharmaceutical distribution model is a data object that includes a sequence of one or more instructions.

The process 400 includes calculating a time-varying behavior of at least one parameter of the computer-executable pharmaceutical distribution model to obtain computational results (404). As an example, with respect to FIGS. 1A-1B, the third-party computing system 108 can calculate a time-varying behavior of one or more parameters (for example, an anticipated number of patients, a rate of patient increase, a rate of patient dropout, a cost of therapy, etc.) over one or more periods of time. The period of time can be a scheduled time that the model is to be in effect (for example, over a period of time defined by a particular start and end date for the model), a time between a scheduled start time that the model is take effect and threshold amount of time thereafter (for example, a month after a start date for the model, six months after the start date for the model, a year after the start date for the model, etc.), or the like. The one or more periods of time can include multiple periods of time, such as each month that the model is scheduled to be in effect, each year the model is to be in effect (for example, first, second, and third years following a start date for a model having a duration of three years), or the like.

The process 400 includes quantifying a value of a first performance metric for the computational results (406).

The process 400 includes adjusting the plurality of distribution constraints to form a computer-executable adjusted pharmaceutical distribution model that includes an adjusted plurality of distribution constraints (408).

The process 400 includes further calculating a time-varying behavior of at least one further parameter of the computer-executable adjusted pharmaceutical distribution model to obtain adjusted computational results (410).

The process 400 includes further quantifying a value of a second performance metric for the adjusted simulation results (412).

The process 400 includes making the computer-executable Adjusted pharmaceutical distribution model accessible to a third-party computing device (414).

FIG. 4B is a flow chart depicting an example process 420 for distributing a pharmaceutical therapy. One or more of the steps, such as each of the steps, of the process 420 may be performed by one or more computers or computing systems. As an example, the process 420 is performed by the third-party computing system 108 (for example, third-party system with respect to the distributor computing system 102 and the supplier computing system 104) shown in FIGS. 1A-1B described above. As another example, the process 420 is performed by the negotiation engine 508 shown in FIG. 5 described below.

The process 420 includes generating a mathematical program that comprises a first objective function and multiple distribution constraints (422).

The process 420 includes solving the mathematical program to generate a computer-executable pharmaceutical distribution model (424).

The process 420 includes performing a dynamic simulation of the computer-executable pharmaceutical distribution model (426).

The process 420 includes calculating a value of a second objective function for the simulation results (428).

The process 420 includes making the computer-executable pharmaceutical distribution model accessible to a third-party computing device (430).

FIG. 4C is a flow chart depicting an example process 440 for distributing a pharmaceutical therapy. One or more of the steps, such as each of the steps, of the process 440 may be performed by one or more computers or computing systems. As an example, the process 440 is performed by the third-party computing system 108 (for example, third-party system with respect to the distributor computing system 102 and the supplier computing system 104) shown in FIGS. 1A-1B described above. As another example, the process 440 is performed by the negotiation engine 508 shown in FIG. 5 described below.

The process 440 includes generating a pharmaceutical distribution model that comprises a vector of distribution constraints and a vector of parameters (442).

The process 440 includes assigning a matrix of values to the vector of parameters (444).

The process 440 includes further assigning a distribution metric to the pharmaceutical distribution model (446).

The process 440 includes simulating the pharmaceutical distribution model using the matrix of values as an input to obtain at least one measure of the distribution metric (448).

The process 440 includes fixing a value for a parameter in the vector of parameters based on the at least one metric to form a reduced-dimensionality pharmaceutical distribution model (450).

The process 440 includes transmitting the reduced-dimensionality pharmaceutical distribution model to a second entity (452).

FIG. 5 shows a block diagram of an exemplary system 500, in accordance with one or more embodiments of the disclosure. System 500 may include m distributor computing devices 502A-502m, n supplier (for example, pharmaceutical company) computing devices 504A-504n, and o third parties 510A-5100. In some implementations, each computing device 502A-502m is associated with a different distributor, each computing device 504A-504n is associated with a different pharmaceutical company, and each computing device 504A-504n is associated with a different third-party of the third parties 510A-5100. In some implementations, government, non-government, donors, hedge-funds, insurance companies, financial institutions, high net worth individuals, or other intermediaries may be an example of a third party, that may step in to guarantee payment for a distributor from an emerging market. In some implementations, a hedge fund may be an example of a third party, that may be willing to buy some of the risk associated with the negotiation between the distributor and the pharmaceutical company. In some implementations, a data provider may be an example of a third-party that may “bid” to provide the data used to clear the payments between the distributor and the pharmaceutical company. In the example of FIG. 5, m, n, and o may be arbitrarily large finite integers greater than or equal to one. The distributors and the pharmaceutical companies may be connected to a negotiation engine 508 via a network 506. In some implementations, network 506 may be any one of a wireless local area network, local area network, Internet, or any other form of network. In some implementations, the third parties 510A-5100 include the one or more data sources 106 described above with respect to FIGS. 1A-1B.

In some implementations, a distributor computing device of the computing devices 502A-502m shown in FIG. 5 is the distributor computing system 102 as shown in FIGS. 1A-1B described above. For example, the distributor computing device 502A is the distributor computing system 102.

In some implementations, a pharmaceutical company computing device of the computing devices 504A-504n shown in FIG. 5 is the supplier computing system 104 as shown in FIGS. 1A-1B described above. For example, the pharmaceutical company computing device 504A is the supplier computing system 104.

In some implementations, the third parties 510A-5100 include the one or more data sources 106 as shown in FIGS. 1A-1B described above.

The negotiation engine 508 receives and evaluates offers from both parties (the distributors 502A-m and the pharmaceutical companies 504A-n). The negotiation engine 508 may be executed and maintained by a third party not related to the distributors 502A-m or the pharmaceutical companies 504A-n. The third party may provide access to the negotiation engine 508 to the pharmaceutical companies and the distributors. In some implementations, the interface of the negotiation engine 508 provided to the distributors may be different from the interface of the negotiation engine provided to the pharmaceutical companies. In some implementations negotiation engine 508 may be hosted on a web server remotely with respect to the distributors 502A-m and pharmaceutical companies 504A-n.

In some implementations, the negotiation engine may include different modes of operation. In some implementations, the negotiation engine may include more than one environment for the users (distributors 502A-m and pharmaceutical companies 504A-n). A first mode of operation, also known as a sandbox environment, may be a simulation environment. The sandbox environment is able to replicate the interface presented to both the distributors and the pharmaceutical companies. However, access to the sandbox environment is limited to people working at an organization, or people associated with the organization who have been provided access to. The sandbox is only a trial environment and trades proposed in the sandbox environment are only for test purposes and have no consequence.

A second mode of operation, also known as terminal environment, is a real-world environment that may result in binding trades. The terminal environment allows users to express commitments that may be presented to other organizations that may be invited to participate in the negotiation process.

Payer and pharmaceutical data and communications between the payers 502A-m and the pharmaceutical companies 504A-n are preferably encrypted.

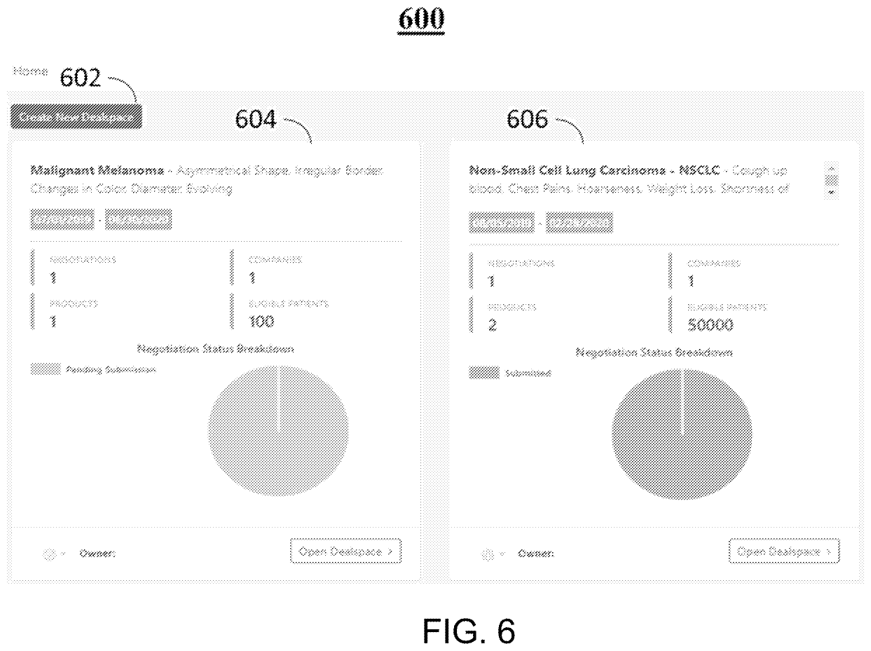

Different facets of the interface of the negotiation engine 508 are described with respect to FIGS. 6-20. Any negotiation, or deal for a drug therapy or regimen, may be initiated from the end of the distributor, through the distributor interface of the negotiation engine. In the distributor interface of the negotiation engine, negotiations may be grouped by “indication or disease”, as described in more detail below. For example, a distributor may open a negotiation for multiple myeloma, and regardless of the therapies used, all negotiations for multiple myeloma will be grouped in the same space. Additionally, a negotiation, or deal for a drug therapy or regimen, may be initiated from the end of the pharmaceutical company, through the pharmaceutical company interface of the negotiation engine. In the interface provided to the pharmaceutical company, negotiations may be grouped by the drugs provided. For example, a pharmaceutical company may group any or all negotiations involving a particular drug (for example, Keytruda), regardless of the diagnosis for which they might be prescribed. The distributors specify negotiation parameters such as contract period, structure, regimen, currency, and pricing units, which are described in greater detail below.

FIG. 6 depicts an exemplary interface 600 of the negotiation engine 508 provided to the distributor, in accordance with one or more embodiments of this disclosure. Any one of the distributors 502A-m are able to access the negotiation engine 508 using credentials provided to them by the third party that is responsible for managing the negotiation engine 508. Once the distributor (for example, distributor) logs into the negotiation engine 508, an interface similar to the interface 600 is presented to the distributor. The interface 600 includes a button 602 that may be used to initialize a new model space. Interface 600 also depicts summaries 604 and 606 of all of the model spaces that are in various stages of progress. A model space is a collection of ongoing negotiations that have been initiated for a particular diagnosis. As shown in FIG. 6, model space summary 604 depicts deals that are in progress for Malignant Melanoma. Model space summary 604 depicts the number of negotiations in progress, the number of companies involved, the number of products in the negotiation, and the number of eligible patients. Similar to model space summary 604, model space summary 606 depicts a summary of ongoing negotiations for Non-Small Cell Lung Carcinoma.

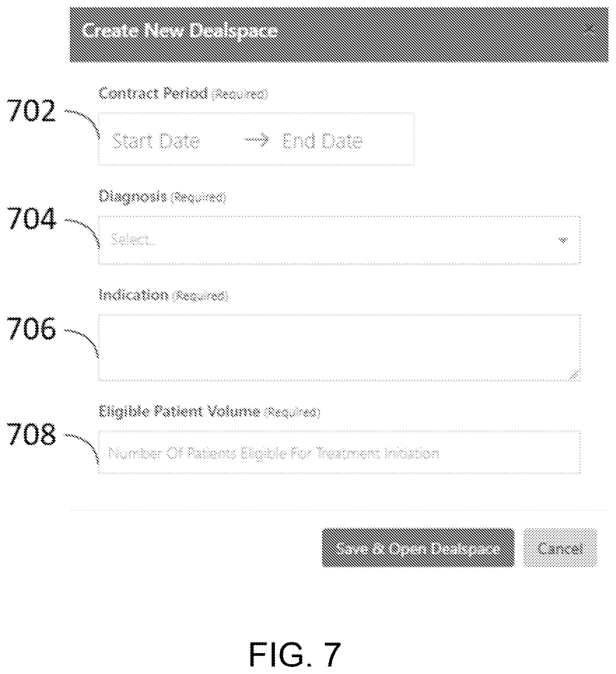

FIG. 7 depicts an exemplary interface 700 of creating a new model space in the negotiation engine, in accordance with one or more embodiments of this disclosure. The interface 700 depicted in FIG. 7 is generated when a user clicks on button 602 in interface 600 of FIG. 6 to create a new model space. In order to generate a new model space, the negotiation engine 508 requires certain information that is considered to be the cornerstone of the negotiations. First, the distributor should define contract period 702 of the negotiation. In some implementations, the start and end dates of the contract period 702 may be selected using a calendar functionality implemented in the negotiation engine 508. Second, the distributor should select a diagnosis for which a drug therapy is required. In some implementations, the diagnosis may be selected from the drop-down menu 704 available in the interface 700. In some implementations, the diagnoses available in the drop-down menu 704 may be the only diagnoses that are offered by the negotiation engine 508 for proposing deals. In addition to the diagnosis, the distributor is required to provide an indication for the diagnoses. The indication may be input in a free text form in the textbox 706 of the interface 700. Finally, the distributor is required to enter the eligible patient volume in textbox 708. In some implementations, the eligible patient volume is defined as the number of patients that are eligible to receive the treatment for the diagnosis selected from the drop-down menu 704. Each of the fields 702, 704, 706, and 708 are required, and the negotiation engine 508 will not proceed to create the relevant model space if any of the required fields are missing.

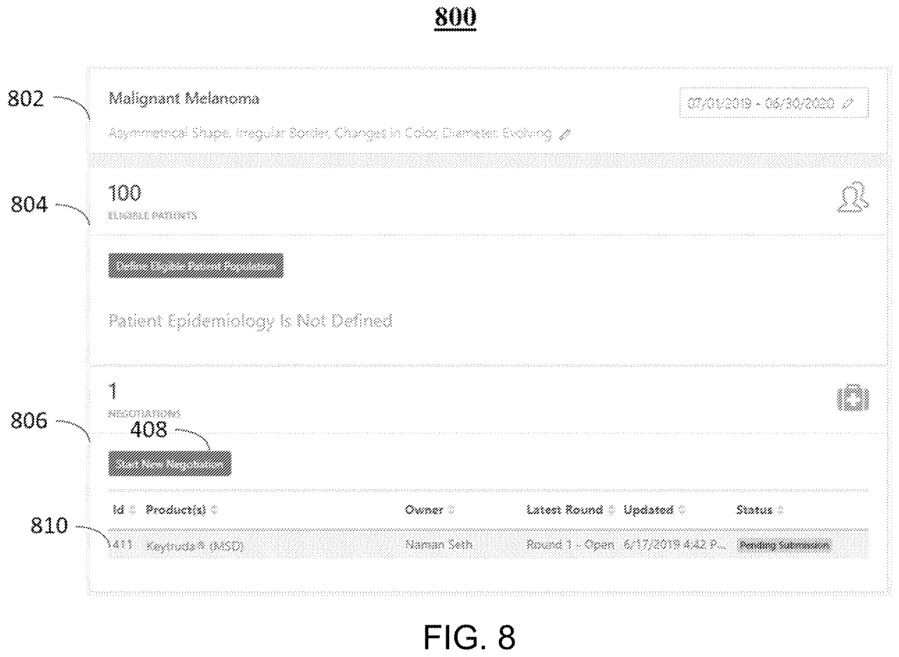

FIG. 8 depicts an exemplary interface 800 of a model space of the negotiation engine, in accordance with one or more embodiments of the disclosure. Interface 800 of the negotiation engine 508 depicts the model space of Malignant Melanoma, the summary of which is depicted as 604 in interface 600 of FIG. 6. The model space interface 800 includes different sections. Section 802 is a header of the model space. Section 802 includes some of the basic information specified in the model space window 700 of FIG. 7. Section 802 includes a diagnosis, contract period, and indication of the diagnosis as specified at the time of the creation of the model space. Section 804 displays the number of patients eligible to receive treatment for the diagnosis specified. In section 804, the distributor has an option to further define the criteria for how the number of eligible payments is determined. In some implementations, this definition may also include characteristics of the patients eligible for the drug therapy.

Section 806 depicts the negotiations in progress for the drug therapies to treat the diagnosis and indications specified in section 802. Section 806 also includes a button 808 that is used to initiate a negotiation with the relevant parties for a drug therapy for the diagnosis. Sub-Section 810 of section 806 includes a list of negotiations for the drug therapy with the relevant parties. For each negotiation in the section 810 that is listed in section 806, there is a name of the product, the round of the negotiation, and a status flag for the negotiation. In some implementations, a round of negotiation is comprised out of the individual versions of the contingent commitments of the negotiating counter-parties.

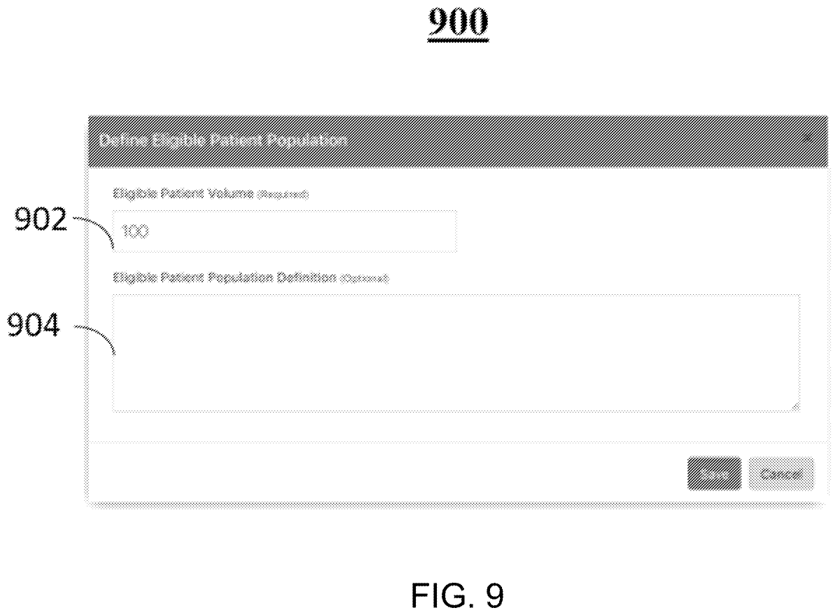

FIG. 9 depicts an exemplary interface 900 of specifying eligible patient population, in accordance with one or more embodiments of this disclosure. The interface 900 includes a text box 902 receives an input of the number of eligible patients from the distributor. The distributor also has an opportunity to provide additional information regarding the eligible patient volume in text box 904. In some implementations, the additional information may include common characteristics of the eligible patients and definitions of the criteria that were used to reach the eligible patient volume in text box 904.

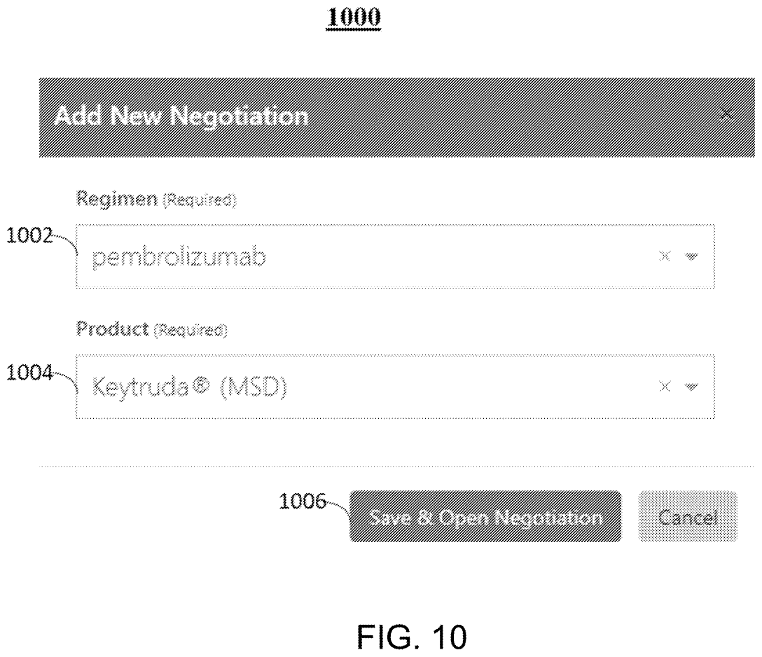

FIG. 10 depicts an exemplary interface 1000 to begin a new negotiation in the negotiation engine, in accordance with one or more embodiments of this disclosure. The interface 1000 of FIG. 10 includes a first field 1002 in which a treatment regimen is specified for the diagnosis. In some implementations, the treatment regimen may be selected from a plurality of treatment regiments available from a drop-down menu. Based on the regimen selected in field 1002, the underlying product combination will appear automatically in the field 1004. In some implementations, if there is more than one product combination available per regimen (for example, through availability of various biosimilars of the main product), the specific product combination will have to be selected via the dropdown of field 1004. Both fields 1002 and 1004 are required. Once the required fields have been populated, the distributor may open a negotiation by clicking on button 1006.

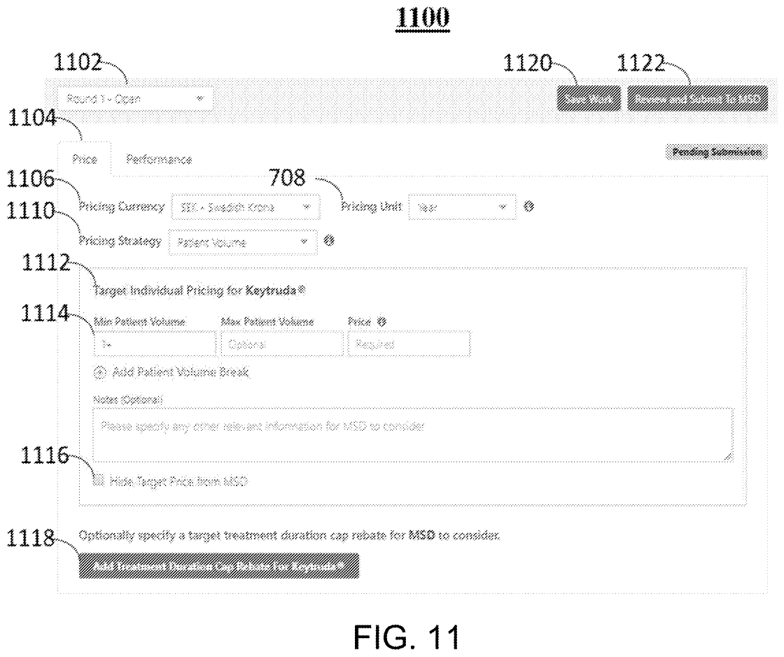

FIG. 11 depicts an exemplary interface 1100 presented to the distributor to begin a new negotiation, in accordance with one or more embodiments of the disclosure. The interface 1100 of FIG. 11 includes a pricing tab 1104. The pricing tab 1104 includes a pricing currency drop-down menu 1106, a pricing unit drop-down menu 1108, and a pricing strategy drop-down menu 1110. Pricing currency drop-down menu 1106 includes all relevant currencies, any one of which may be selected for the negotiation.

In some implementations, price levels of the drugs (for example, ex-factory, pharmacy purchasing price, etc.) may have to be agreed on for each negotiation. Pricing unit drop-down menu 1108 includes a plurality of options, for example, Year Price, Quarter Price, Month Price, and Cycle Price. Cycle Price reflects the treatment of a single patient for one cycle. In some implementations, one cycle of treatment for a patient is 3 weeks. There might be a 4th week where the patient does not consume any medication. In some implementations, as the cycle progresses, the dosage of the medication may change. Month Price reflects the treatment of a single patient for one month. Quarter Price reflects the treatment of a single patient for one quarter. Year Price reflects the treatment of a single patient for one year.

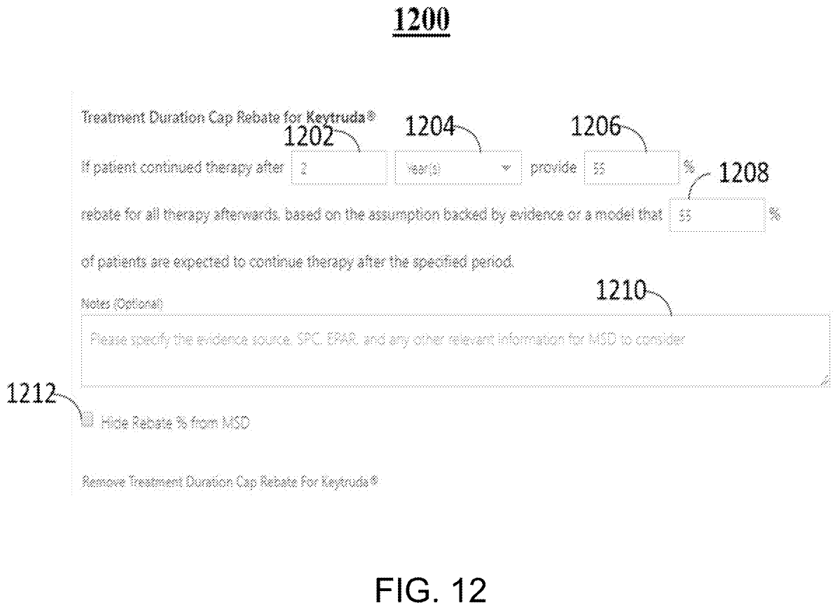

Pricing strategy drop-down menu 1110 includes different patient strategies for pricing. In some implementations, the distributor may add a preference for a particular therapy over others, and the interface may allow the distributor to specify multiple preferences. A first strategy of pricing is Patient Volume. Patient Volume Pricing is based on the number of patients treated. Because Patient Volume Pricing is based on the number of patients, price provisions may differ based on the total volume of patients treated. Section 1112 depicts an interface for pricing details for the Patient Volume. At 1114, the distributor may specify a minimum patient volume, a maximum patient volume, and a price point for the patient volume. In some implementations, if price doesn't change depending on volume, the max patient volume field can be left empty. In some implementations, the distributor may wish to break down the pricing patient volume. In such cases, the distributor may enter patient volume breaks and corresponding new price for volume dependent pricing proposals. At 1116, the distributor has the option to “Hide Target Price” from the pharmaceutical company. In some implementations, the option to hide the target price may be a global setting or an individual setting. For example, in some countries all data, or specific data will always be hidden, or always visible, and in other countries the distributors or pharmaceutical companies may be free to choose what they want to do. Selection of this option will hide the target (combination) price will be hidden from the other negotiation parties. In some implementations, an additional provision in the Patient Volume pricing provision may be defined. This additional provision may be a “Treatment Duration Cap Rebate.” The interface for the “Treatment Duration Cap Rebate” may be initiated by pressing the button 1118. This treatment duration cap rebate allows for additional provisions on top of volume provisions (e.g. in the case of expected market share uncertainty). The interface is described in more detail in FIG. 12.

A second pricing strategy in the drop-down menu 1110 is a Treatment Duration Price pricing strategy. A Treatment Duration Pricing Strategy is based on how long a patient is treated. As a consequence, the price of the treatment strategies will be different for each patient based on how long they have been in treatment. In some implementations, duration of the treatment may be broken down into Year, Quarter, Month, and Cycle. In addition to volume breaks, that is also available in the Patient Volume pricing strategy, allows for an option of duration break.

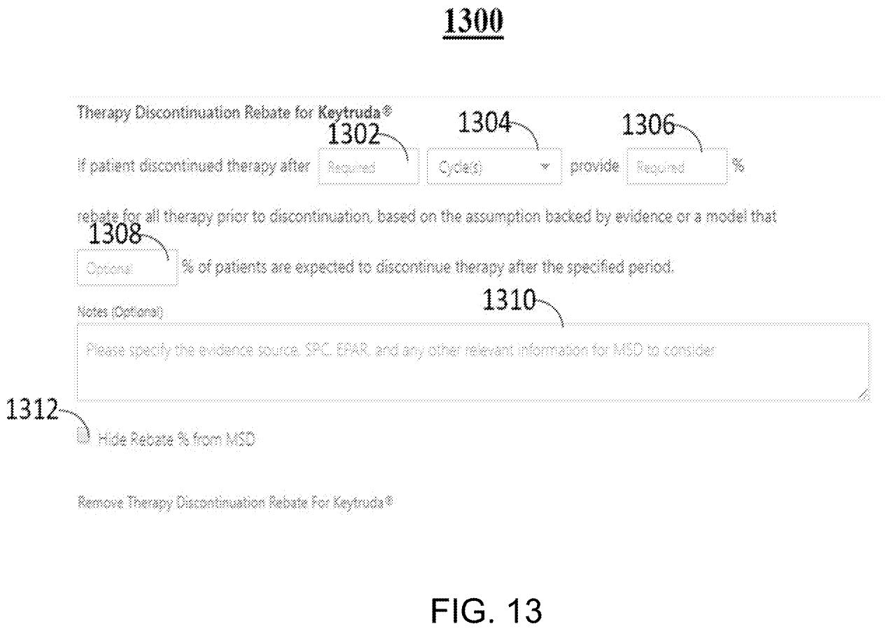

The interface 1100 of FIG. 11 includes a performance tab next to the pricing tab 1104. The performance tab specifies price provisions can be made based on (treatment) performance. The interface for the performance tab is described in more detail in FIG. 13.

FIG. 12 depicts an exemplary interface 1200 of a “Treatment Duration Cap Rebate” as initiated from interface 1100 of FIG. 11, in accordance with one or more embodiments of the disclosure. The interface 1200 of FIG. 12 receives input that requests price rebates (from 1-100%) for patients in input field 1206, based on the evidence that a certain proportion of the patients specified in input field 1208 are expected to continue therapy after the specified period specified in input fields 1202 and 1204. The distributor has an option to specify to define their inputs received in interface 1200 in the “Notes” section 1210. The distributor also has the option of hiding this rebate specified from the pharmaceutical company by selecting option 1212.

FIG. 13 depicts an exemplary interface 1300 of “Therapy Discontinuation Rebate” as initiated from interface 1100 of FIG. 11, in accordance with one or more embodiments of the disclosure. The therapy discontinuation rebate interface is initiated from the Performance tab of interface 1100 of FIG. 11. In some implementations, pricing provisions may be made based on the performance of the drug regimen. The interface 1300 of FIG. 13 receives input that requests price rebates (from 1-100%) for patients in input field 1306, based on the evidence that a certain proportion of the patients specified in input field 1308 are expected to discontinue therapy due to side effects/toxicity or lack of response to a specific regimen after the specified period specified in input fields 1302 and 1304. The distributor has an option to specify to define their inputs received in interface 1300 in the “Notes” section 1310. The distributor also has the option of hiding this rebate specified from the pharmaceutical company by selecting option 1312.

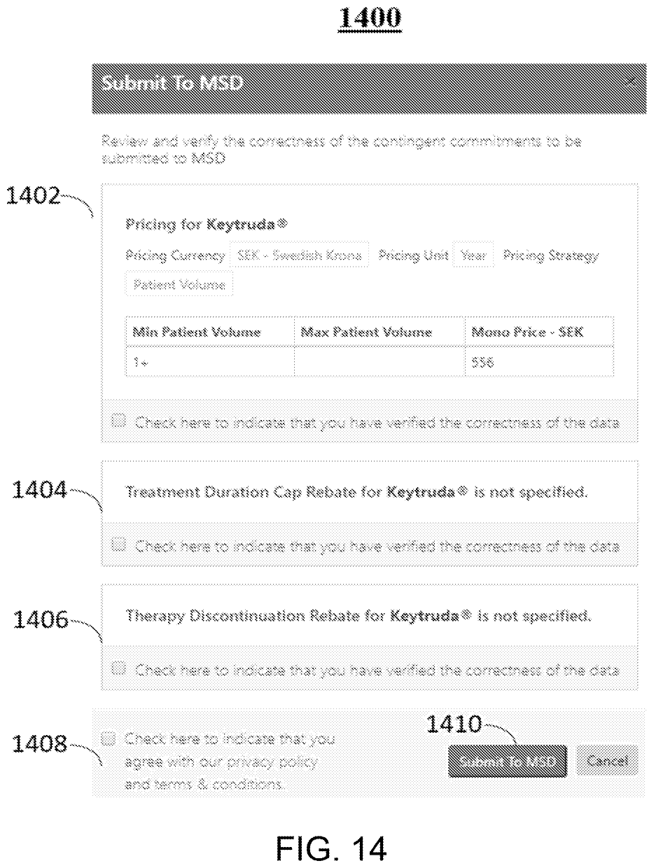

Once the distributor (for example, the distributor) has specified all the required information in the interfaces presented in FIG. 6-13, the distributor may submit his proposal to the pharmaceutical company for consideration. The distributor may begin the submission by pressing the submit button 1122 in interface 1100 of FIG. 11. Before the distributor submits the contingent commitments, a pop-up window similar to the interface 1000 of FIG. 14 will be displayed to the distributor.

FIG. 14 is an exemplary interface 1400 depicting a summary of the deal proposal before it is submitted to the pharmaceutical company, in accordance with one or more embodiments of the disclosure. The interface 1400 of FIG. 14 is divided into four sections, each of which summarize the inputs provided in the interfaces depicted by FIGS. 11-13, and a privacy agreement. Section 1402 summarizes the pricing strategy, pricing unit, and the pricing currency as specified in the interface of FIG. 11. Section 1402 includes the breakdown by patient volume, if specified by the distributor interface 1100 of FIG. 11. Section 1404 includes the “Treatment Duration Cap Rebate” if specified in interface 1200 of FIG. 12. Section 1406 includes the “Therapy Discontinuation Rebate,” if specified in interface 1300 of FIG. 13. For each section 1402, 1404, and 1406, a check-box is present to ensure that the correctness of the data has been verified. Once the data presented in each section has been verified and the privacy agreement in section 1408 has been acknowledged, the pricing proposal may be submitted to the other negotiating parties.

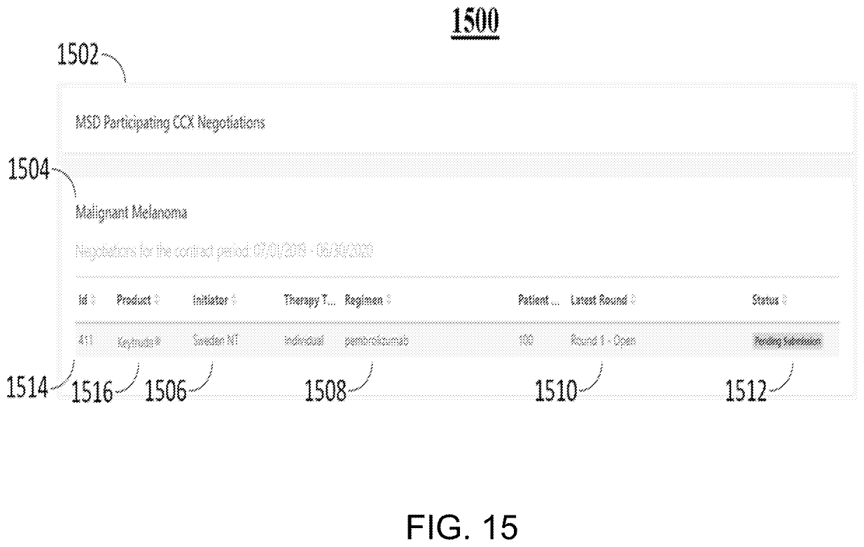

FIG. 15 depicts an exemplary interface 1500 of the negotiation engine provided to a pharmaceutical company, in accordance with one or more embodiments of this disclosure. Any one of the pharmaceutical companies 504A-n are able to access the negotiation engine 508 using credentials provided to them by the third party that is responsible for managing the negotiation engine 508. Once the pharmaceutical company logs in to the negotiation engine 508, an interface similar to interface 1500 is presented to the pharmaceutical company. The interface 1500 has a header 1502 that depicts the negotiations that the pharmaceutical company is involved in. Section 1504 organizes by diagnosis, the negotiations that the pharmaceutical company is invited to participate in. As shown in FIG. 15, record 1514 summarizes a negotiation proposed by an initiator 1506 for a diagnosis specified by the record 1514. Record 1514 depicts a negotiation for a product 1516 initiated by the initiator 1506. The record 1514 also specifies the regimen 1508, the number of rounds 1510 the negotiation has gone through, and the current status of the negotiation 1512.

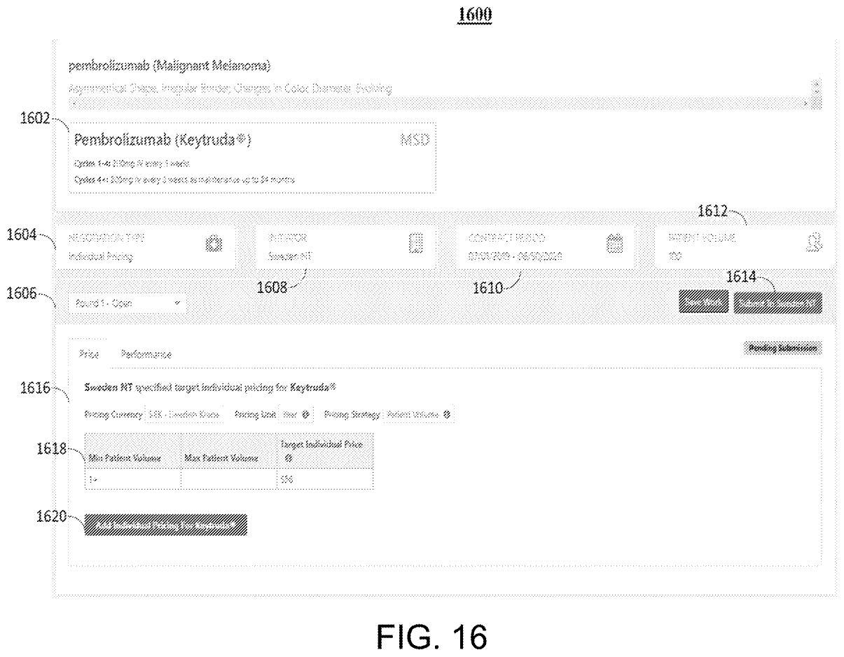

FIG. 16 depicts an exemplary interface 1600 presented to the pharmaceutical company to respond to a proposed negotiation, in accordance with one or more embodiments of the disclosure. The interface 1600 of FIG. 16 includes a section 1602 that specifies the drug regimen that is the subject of the proposed negotiation. Section 1604 of the interface specifies the type of the proposed negotiation. Section 1608 specifies the initiator of the negotiation, section 1610 specifies the contract period for the negotiation, and section 1612 specifies the number of patients that might be eligible for the drugs. Section 1606 depicts the proposal from the distributor. Subsection 1616 of section 1606 depicts the pricing proposed by the initiator of the negotiation, along with the pricing currency, pricing unit, and pricing strategy. Table 1618 of the subsection 1616 specifies the pricing by patient volume as specified by the distributor.

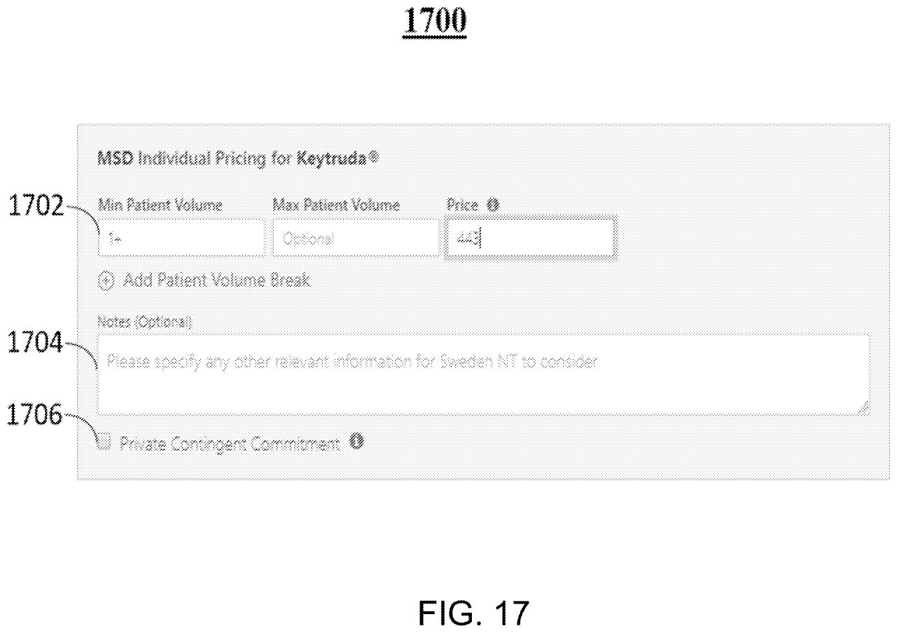

Subsection 1616 has a button 1620 that allows the pharmaceutical company to add their own individual pricing for the drug regimen of the proposed negotiation. The interface of adding individual pricing is described in more detail in FIG. 17. Once the pharmaceutical company is satisfied by their specified individual pricing, the pharmaceutical company is able to submit their pricing to the distributor by clicking the button 1614.

FIG. 17 depicts an exemplary interface 1700 for specifying a pricing strategy of the pharmaceutical company in response to the proposal from the distributor. At an interface element 1702, the pharmaceutical company may specify a minimum patient volume, a maximum patient volume, and a price point for the patient volume. In some implementations, if price doesn't change depending on volume, the max patient volume field can be left empty. In some implementations, the distributor may wish to break down the pricing patient volume. In such cases, the distributor may enter patient volume breaks and corresponding new price for volume dependent pricing proposals. In textbox 1704, the pharmaceutical company may justify the breakdown of their pricing model. At an interface element 1706, the distributor has the option to hide the pricing model from the distributor, and other parties involved in the negotiation.

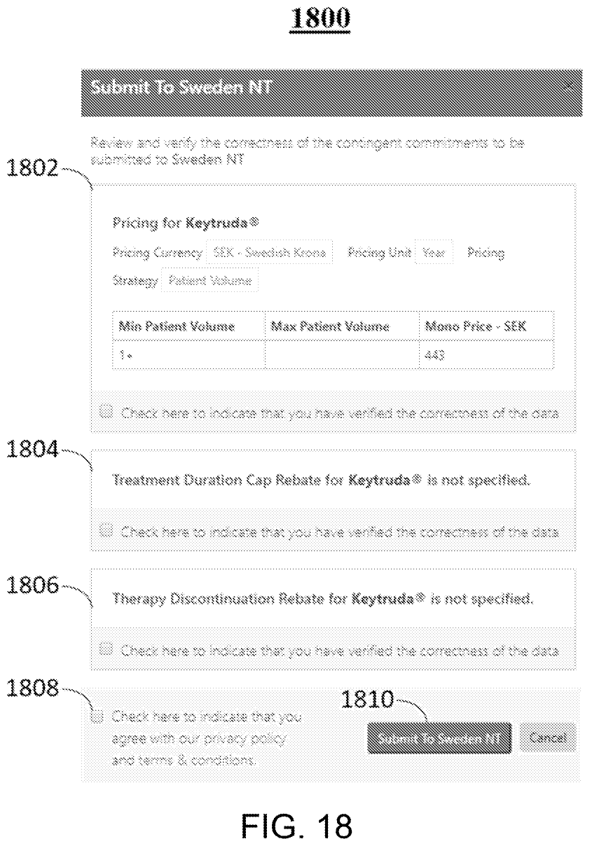

FIG. 18 depicts an exemplary interface 1800 depicting a summary of the deal proposal before it is submitted to the distributor, in accordance with one or more embodiments of the disclosure. Once the pharmaceutical company has specified all the required information, the pharmaceutical company may submit his proposal to the distributor for consideration. The pharmaceutical company may begin the submission by pressing the submit button 1614 in interface 1600 of FIG. 16. Before the pharmaceutical company submits the pricing model, a pop-up window that is, for example, the interface 1800 of FIG. 18 will be displayed to the pharmaceutical company. FIG. 18 is an interface depicting a summary of the deal proposal before it is submitted to the distributor, in accordance with one or more embodiments of the disclosure. Interface 1800 of FIG. 18 is divided into four sections, each of which summarize the inputs provided as part of the pricing model, and a privacy agreement. Section 1802 summarizes the pricing strategy, pricing unit, and the pricing currency as specified in the interface of FIG. 17. Section 1802 includes the breakdown by patient volume, if specified by the distributor interface 1700 of FIG. 17. Section 1804 includes the “Treatment Duration Cap Rebate” if specified as part of the pricing model. Section 1806 includes the “Therapy Discontinuation Rebate,” if specified as part of the pricing model. For each section 1802, 1804, and 1806, a check-box is present to ensure that the correctness of the data has been verified. Once the data presented in each section has been verified and the privacy agreement in section 1808 has been acknowledged, the pricing proposal may be submitted to the other negotiating parties through the selection of the interface element 1810 (for example, button).

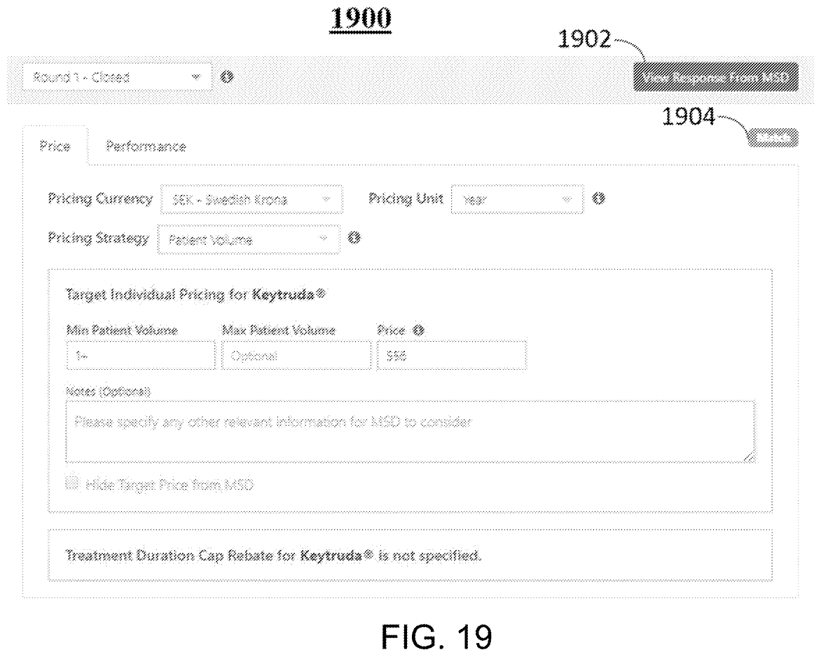

FIG. 19 depicts an exemplary interface 1900 presented to the distributor during a negotiation, in accordance with one or more embodiments of the disclosure. The interface 1900 of FIG. 19 is a modified version of the interface 1100 of FIG. 11. Once all the parties involved in a negotiation have entered their proposals, the first round of negotiation is considered closed. Interface 1900 includes an indicator 1904 that displays an evaluation of the proposals to determine whether there is an overlap between the proposals, or there is no match between the proposals. In some implementations, when there is no match between proposals, the interface 1900 of negotiation engine 508 may include a color scale (red yellow green) indicator in place of indicator 1904 with a slider of where the negotiation stands as far as likelihood of success. In some implementations, the color scale may not be a single color, but a scale (more like an analog thermometer instead of a digital one). changing different elements of the negotiation (price, volume, performance provisions, etc.) could have an impact on where the marker is on the scale and change it at different rates. For example, the thresholds may have a randomly generated fudge factor, so negotiation parties may not figure out what is happening in the background. If the distributor's price is $100 and the pharmaceutical company's price is $110, then the color scale in the interface of the pharmaceutical company of the negotiation engine 508 may be green (based on the assumption both will go down in price). Similarly, if the pharmaceutical company lists its price as $120 then the color scale is yellow, and if the pharmaceutical company's price is $130, the color scale is red.

In some implementations, if the initiator of the negotiation is the distributor, the interface 1900 of the negotiation engine 508 shows a pricing recommendation section (not shown) in a pricing commitment tab above or below the pricing provision. In some implementations, the pricing recommendation section includes one or multiple price tiers along with the graphical representation of a probability of success. In some implementations, the probability of success may be based on a contingent commitment specified by the distributor. For example, the contingent commitment may specify a limit of a matching percentage, or a percentage of other's price. In some examples, the contingent price may be based on dollar amounts. In some examples, the initiator of a negotiation may have the ability to introduce a commitment based on commitments from the opposing party of the negotiation. In some implementations, in case the negotiation engine 508 determines that the pricing specified in the previous round should not change “price should not be changed” will appear along with a probability of success widget, in the pricing commitment tab, if the distributor keeps the same price. The probability of success could be different from the previous round with the assumption that there is a certain probability that the pharmaceutical company will accept the nudging recommendation.

In some implementations, if the initiator of the negotiation is distributor the interface 0f the negotiation engine 508 associated with the distributor shows a treatment continuation recommendation section in the Duration Cap tab above or below the treatment continuation commitment. In some implementations, this would include information such as number of cycles after which the treatment continuation rebate applies, rebate percentage, percentage of the patient population expected to continue the therapy, and a probability of success widget. In some implementations, if the nudging algorithm of the negotiation engine 508 determines that the treatment continuation information specified in the previous round should not change, in the Duration Cap tab “treatment continuation commitment should not be changed” message will appear along with the probability of success widget. In some implementations, the probability of success could be different from the previous round with the assumption that there is a certain probability that the distributor will accept the nudging recommendation. In some implementations, the nudging algorithm of the negotiation engine 508 will determine that the therapy continuation commitment should be removed and incorporated into the pharmaceutical company's pricing commitment. In this case an adequate message would be displayed to the pharmaceutical company on both Pricing and treatment Duration Cap tabs along with the probability of success widget.

In some implementations, if the initiator of the negotiation is distributor, the interface of the negotiation engine 508 shows a therapy discontinuation recommendation section in the Performance Tab above or below the therapy discontinuation commitment. In some implementations, this would include information such as number of cycles after which the therapy discontinuation rebate applies, rebate percentage, percentage of the patient population expected to discontinue the therapy, probability of success widget. In some implementations, if the nudging algorithm of the negotiation engine 508 determines that the therapy discontinuation information specified in the previous round should not change, in the Performance Tab “therapy discontinuation commitment should not be changed” message will appear along with the probability of success widget. In some implementations, the probability of success could be different from the previous round with the assumption that there is a certain probability that the distributor will accept the nudging recommendation. In some implementations, it is possible that the nudging algorithm will determine that the therapy discontinuation commitment should be removed and incorporated into the pharmaceutical company's pricing commitment. In some implementations, an adequate message would be displayed to the pharmaceutical company on both pricing and performance tabs along with the probability of success widget.

In some implementations, the nudging algorithm of the negotiation engine 508 would detect the distributor should change the pricing strategy or the pricing unit in the next round. The distributor interface will display such message to the user just below the Round section of the screen.

In some implementations, the probability of success widget described at the round level displays an overall success probability of the round. In some implementations, the probability of success widget at the commitment level i.e. pricing/therapy continuation/therapy discontinuation is a percentage widget which displays a delta success factor each commitment recommendation attributes to the overall negotiation success probability.

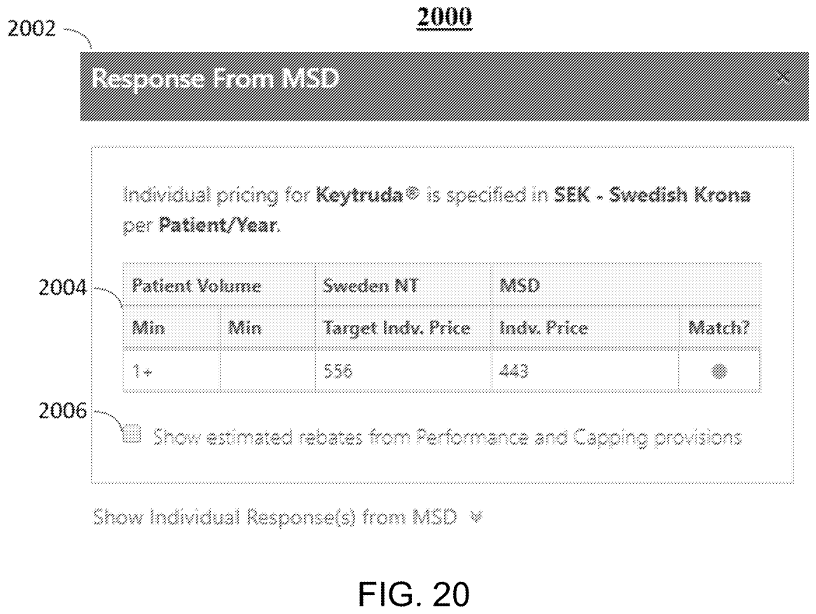

The platform will indicate/whether a match has been achieved or not (based purely on price). It is possible that a price is acceptable to all parties, but includes price provisions which depend on actual patient treatment durations and responses. More information regarding the price provided by the other involved parties is available if the distributor decided to click the button 1902. The interface for the pricing model provided by the pharmaceutical company is described in more detail in FIG. 20.

In case there is no match, the negotiation engine 508 will distinguish between deal-components where there might be a match and deals where there is no match. In some implementations, the negotiation engine will nudge both the parties to a more equitable solution.

FIG. 20 depicts an exemplary interface 2000 that displays the pharmaceutical companies pricing model to the distributor, in accordance with one or more embodiments of the disclosure. The interface 2002 may be generated when the distributor clicks on button 1902 of interface 1900 of FIG. 19 to view the response from the pharmaceutical company to the proposal of the distributor. Interface 2002 displays a table 2004 that details the basic pricing model proposed by the pharmaceutical company. Furthermore, the distributor may generate a second interface (not pictured) to view the rebates specified by the pharmaceutical company.

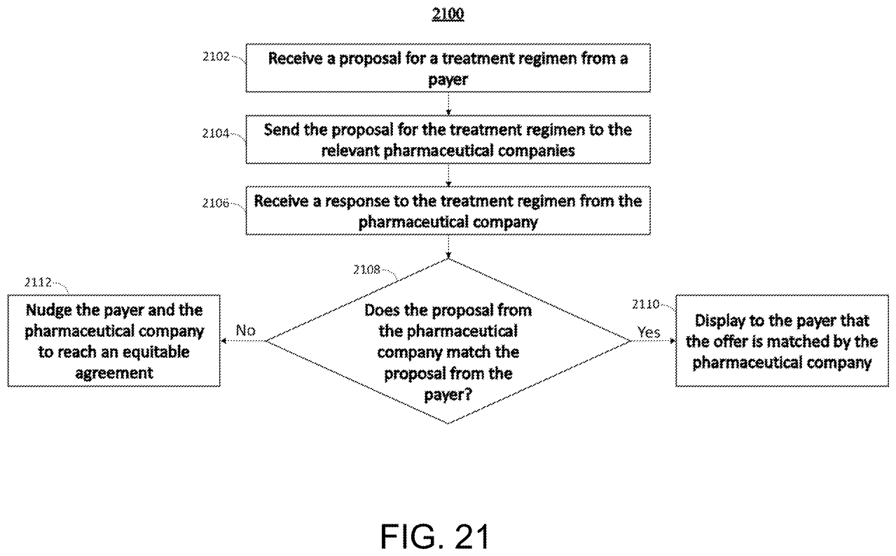

FIG. 21 depicts an illustrative flowchart of a process 2100 of a negotiation between two parties, in accordance with one or more embodiments of the disclosure. One or more of the steps, such as each of the steps, of the process 2100 may be performed by one or more computers or computing systems. As an example, the process 2100 is performed by the negotiation engine 508 shown in FIG. 5 described above. As another example, the process 2100 is performed by the third-party computing system 108 (for example, third-party system with respect to the distributor computing system 102 and the distributor computing system 104) shown in FIGS. 1A-1B described above.

At 2102, the negotiation engine 508 receives a proposal for a treatment regimen for a diagnosis from a distributor.

At 2104, the negotiation engine 508 transmits the received proposal to the relevant pharmaceutical companies that have products that are part of the treatment regimen.

At 2106, the negotiation engine 508 receives a response to the proposal for a treatment regimen for a diagnosis from the relevant pharmaceutical companies.

At decision block 2108, the negotiation engine 508 determines whether the proposal from the pharmaceutical company matches the proposal from the distributor. In response to determining that the proposal from the pharmaceutical company matches the proposal from the distributor, the negotiation engine 508 proceeds to 2110 to display to the distributor that the offer of the distributor is matched by the relevant pharmaceutical companies. In response to determining that the proposal from the pharmaceutical company matches the proposal from the distributor, the negotiation engine 508 proceeds to 2112 to a nudging mode where the negotiation engine nudges the distributor and the pharmaceutical company to reach an equitable agreement. In some implementations, the negotiation engine 508, in the nudging mode, recommends a course of action to each party that may get the deal closed without revealing anything material about the opposing party's confidential threat points. In some implementations, the recommendation provided by the negotiation engine 508 begins a new round of negotiation. In such embodiments, as the recommended course action is based on information from both parties, the recommended course of action is not reasonably unbiased to any one party to the exchange. In some implementations, the recommendations are determined based on data from past negotiations available recorded from previous negotiations. In such embodiments, the negotiation engine 508 may introduce new elements to the negotiation that the parties may not have included. For example, “similar deals have been completed in the past when they include a performance provision.”

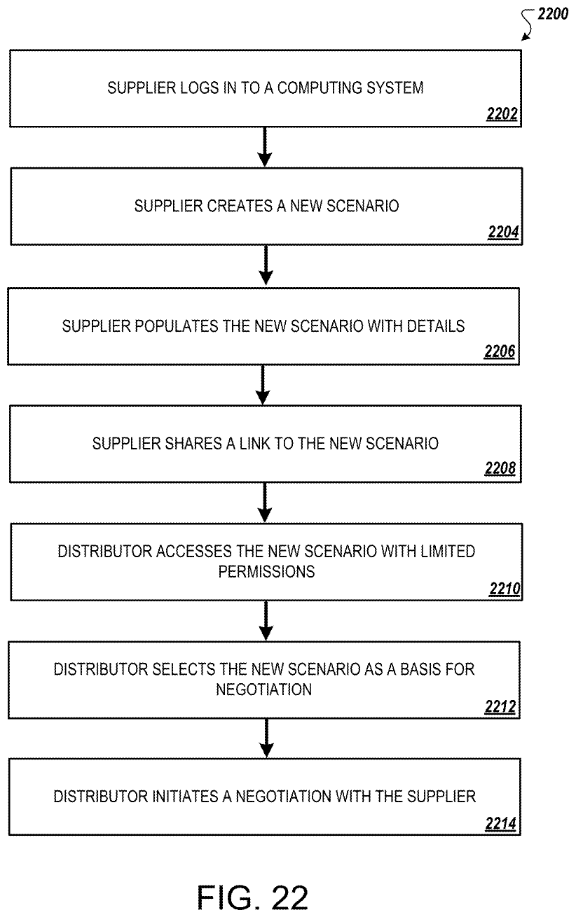

FIG. 22 is a flow chart depicting an example process 2200 for distributing a pharmaceutical therapy. One or more of the steps, such as each of the steps, of the process 2200 may be performed by one or more computers or computing systems. As an example, the process 2200 is performed by the negotiation engine 508 shown in FIG. 5 described above. As another example, the process 2200 is performed by the third-party computing system 108 (for example, third-party system with respect to the distributor computing system 102 and the supplier computing system 104) shown in FIGS. 1A-1B described above.

The process 2200 includes a supplier logging in to a computing system (2202). For example, with respect to FIGS. 1A-1B, a user of the supplier computing system 104 logs into the third-party computing system 108 to access a sandbox environment hosted on the third-party computing system. As another example, with respect to FIG. 5, a user of the supplier computing device 504A logs into a sandbox environment provided by the negotiation engine 508.

The process 2200 includes the supplier creating a new scenario (2204). For example, with respect to FIGS. 1A-1B, the third-party computing system 108 presents a user of the supplier computing system 104 an interface to create a new scenario for the distribution of a pharmaceutical therapy (for example, the interface 200 shown in FIG. 2A, the interface 300 shown in FIG. 3A, etc.). As another example, with respect to FIG. 5, the negotiation engine 508 presents a user of the supplier computing device 504A an interface to create a new scenario for the distribution of a pharmaceutical therapy.

As an example, the third-party computing system 108 shown in FIGS. 1A-1B or the negotiation engine 508 shown in FIG. 5 presents a user of a supplier computing system or device with an interface that includes a left panel area and a right panel area. As an example, the left panel area can include five columns and provide an analysis tool, and the right panel can include seven columns and provide one or more bid entry features and/or contract structure features. In response to a user entering details in the right panel area, for example, the third-party computing system 108 shown in FIGS. 1A-1B or the negotiation engine 508 shown in FIG. 5 may automatically populate and/or update information presented in the left panel area.

The process 2200 includes the supplier populating the new scenario with details (2206). As an example, these details can include defining constraints for the new scenario, assigning values to parameters for the new scenario, or a combination of the two. As another example, these details can include one or more of the following: one or more pharmaceutical drugs, a dosage of one or more pharmaceutical drugs, a condition (for example, diagnosis) to be treated, a region for distribution of a therapy, an agreement duration or duration range, a preferred start date for the agreement, a number of rounds of negotiation with one or more suppliers of the therapy, a number of patients per year or per duration of the contract, a number of therapy cycles required per patient, a price per cycle/pack/dose of therapy, a price per patient, rebate amounts, or the like. However, providing details can include assigning values to various other parameters, including pricing parameters.

In some implementations, populating the new scenario with details includes structuring the new scenario.

In some implementations, one or more details of the new scenario are automatically populated. As an example, with respect to FIGS. 1A-1B, the third-party computing system 108 may use a translator tool that populates certain details based on information input by the supplier and/or distributor. In more detail, in response to entering details about the diagnosis to be treated and a dosage of a particular pharmaceutical drug of the therapy, the translator tool can automatically populate a value for a parameter of the new scenario for the number of cycles/packs/doses of the required per patient. For example, if the supplier indicates that the condition is a Disease X and the therapy is a 100 mg of Drug A, the translator tool can automatically set a value of 3 for the number of therapy cycles. The translator tool may determine values using one or more lookup tables. Additionally or alternatively, the translator tool may leverage one or more algorithms to calculate values based on inputs provided by the supplier and/or distributor.

In some implementations, the new scenario is updated in real-time as it is populated with details. For example, a translator tool and/or analysis tool can update an interface presented to a supplier in real-time in response to the supplier providing details for the scenario. As an example, in response to supplier providing a diagnosis to be treated and an indication of the drug to be distributed, the translator tool can provide a drop-down menu of available dosages of the drug. In response to the supplier selecting a particular dosage, the translator tool can update the interface to show a number of packs per patient of the drug that are required per patient. Based on this information and in response to the supplier providing a dosage price per therapy pack and an anticipated number of patients per year, the analysis tool can update the interface to present a cost per year for a distributor.

In some implementations, after or during the process of populating the new scenario with details, the new scenario is saved. For example, with respect to FIGS. 1A-1B, the new scenario is saved on the third-party computing device 108.

In some implementations, after or during the process of populating the new scenario with details, the new scenario is compared with one or more other scenarios. For example, with respect to FIGS. 1A-1B, the third-party computing system 108 can recommend to a user of the supplier computing system 104 a list of one or more scenarios (for example, previously finalized pharmaceutical distribution models, scenarios previously generated by the supplier, etc.). If the user selects a scenario from the list, the third-party computing system 108 can update the interface to present the two scenarios side-by-side. Additionally or alternatively, the third-party computing system 108 can provide an interface that highlights or lists the differences between the two scenarios (for example, different assumptions for the scenarios, different parameter values assigned for the two scenarios, different definitions for pharmaceutical therapy distribution constraints of the two scenarios, differences in performance results for the two scenarios, etc.).

The process 2200 includes the supplier sharing a link to the new scenario (2208). As an example, with respect to FIGS. 1A-1B, in response to a request made by user of the supplier computing system 104 to share the new scenario with one or more distributors, the third-party computing system 108 generates a link (for example, URL) and transmits the link to the distributor computing system 102 and/or one or more other distributor computing systems. As another example, with respect to FIG. 5, in response to a request made by user of the supplier computing device 504A to share the new scenario with one or more distributors, the negotiation engine 508 generates a link (for example, URL) and transmits the link to one or more of the distributor computing devices 502A-502m (for example, all the distributor computing devices 502A-502m or a subset of the distributor computing devices 502A-502m).

In some implementations, the link to the new scenario is shared with a subset of available distributors. For example, the link to the new scenario may be shared only with those distributors that operate in a region specified by the new scenario based on details populated by the supplier.

The process 2200 includes the distributor accessing the new scenario with limited permissions (2210). For example, with respect to FIGS. 1A-1B, a user of the distributor computing system 102 logs into the third-party computing system 108 to access the sandbox environment or a different version of the sandbox environment hosted on the third-party computing system 108 to view the new scenario. As another example, with respect to FIG. 5, a user of the supplier computing device 504A logs into the sandbox environment provided by the negotiation engine 508 or a different version of the sandbox environment provided by the negotiation engine 508 to view the new scenario.

In some implementations, the distributor selects the new scenario from a list of multiple scenarios. For example, with respect to FIGS. 1A-1B, after a user of the distributor computing system 102 logs into the third-party computing system 108 to access the sandbox environment or a different version of the sandbox environment hosted on the third-party computing system 108, the third-party computing system 108 presents an interface with a list of multiple available scenarios (for example, scenarios that have been created but are not currently being negotiated, scenarios that have been created and are potentially being negotiated but the negotiations are not finalized, etc.). The user of the distributor computing system 102 can then select the new scenario from the list of multiple available scenarios.

In some implementations, the permissions prevent a distributor from editing the new scenario. For example, with respect to FIGS. 1A-1B, a user of the distributor computing device 102 logs into a sandbox environment hosted by the third-party computing system 108 to view an interface that represents or otherwise displays the details of the new scenario. However, the interface may be locked or static to prevent editing (for example, the interface does not include editable interface elements, the interface presents different interface elements that are typically editable such as interface fields but they are in a locked state to prevent editing, etc.).

In some implementations, the permissions permit the distributor to adjust a subset of details populated in the new scenario. For example, with respect to FIGS. 1A-1B, a user of the distributor computing device 102 logs into a sandbox environment hosted by the third-party computing system 108 to view an interface that represents or otherwise displays the details of the new scenario. The interface may be partially locked or static to permit editing of only a subset of details. As an example, a user of the distributor computing device 102 may be able to edit a field for the agreement duration or be able to enter (or select) a particular value for the duration from a range or list of permitted duration values provided by the supplier. As another example, a user of the distributor computing device 102 may be able to edit a field for the number of patients (for example, anticipated number of eligible patients per year or per contract duration).

The process 2200 includes the distributor selecting the new scenario as a basis for negotiation (2212). For example, with respect to FIGS. 1A-1B, after a user of the distributor computing system 102 views a new scenario in an interface presented by the third-party computing system 108, the user can select an interface element in the interface to indicate that the user would like to use this scenario as a basis for negotiation (for example, with the supplier or with a different supplier).

The process 2200 includes the distributor initiating a negotiation with the supplier (2214). For example, with respect to FIGS. 1A-1B, after a user of the distributor computing system 102 selects an interface element in the interface to indicate that the user would like to use this scenario as a basis for negotiation, the third-party computing system 108 provides the user an option to initialize a negotiation with the supplier. If the user selects the option, the third-party computing system 108 can transmit a notification to the supplier computing system 104 that the new scenario has been selected as a basis of negotiation by the particular distributor having the distributor computing system 102.

In some implementations, the new scenario is shared internally or externally. For example, with respect to FIGS. 1A-1B, the new scenario can be shared internally within the supplier computing system 104 (for example, internal due to the supplier initializing the new scenario). As another example, with respect to FIGS. 1A-1B, the new scenario can be shared externally with one or more distributors (for example, that the supplier believes might be interested in the new scenario). Sharing the new scenario can include sharing a link (for example, URL) for the new scenario that a user of a computing system can access the new scenario through. Sharing the new scenario can include sharing computer-executable program for the new scenario that a user of a computing system can execute on their computing system. Sharing the new scenario can include sharing a document (for example, PDF) for the new scenario that a user of a computing system can view on their computing system.

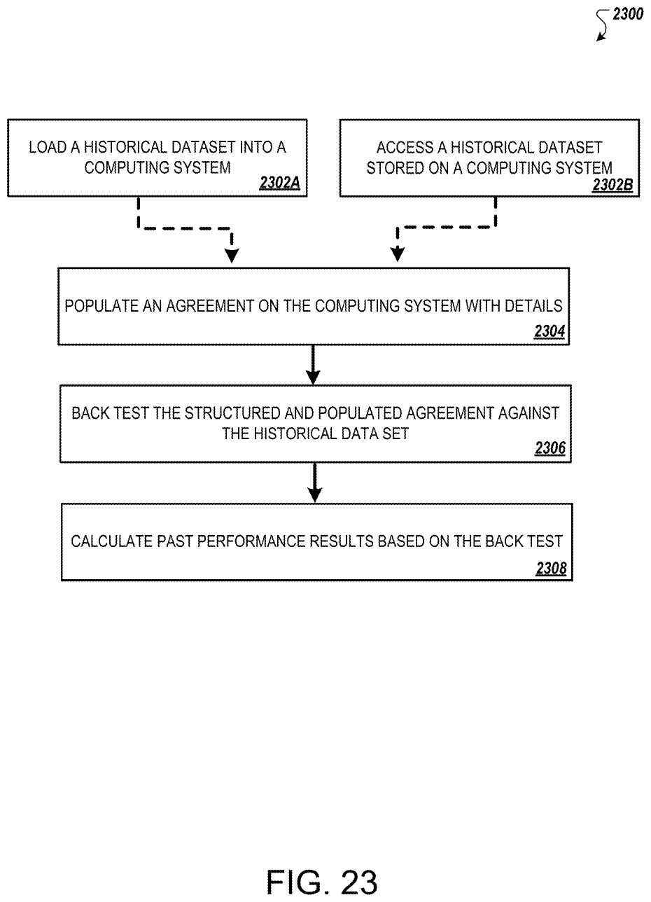

FIG. 23 is a flow chart depicting an example process 2300 for testing a model for distributing a pharmaceutical therapy. One or more of the steps, such as each of the steps, of the process 2300 may be performed by one or more computers or computing systems. As an example, the process 2300 is performed by the negotiation engine 508 shown in FIG. 5 described above. As another example, the process 2300 is performed by the third-party computing system 108 (for example, third-party system with respect to the distributor computing system 102 and the distributor computing system 104) shown in FIGS. 1A-1B described above.

The process 2300 optionally includes loading a historical dataset into a computing system (2302A). For example, with respect to FIGS. 1A-1B, loading a historical dataset can include the third-party computing system 108 obtaining a historical dataset from the one or more data sources 106 and storing the historical dataset on the third-party computing system 108 (for example, as part of the medical data 128). In more detail, the historical dataset can include research data obtained from a university or a government website related to a particular therapy or condition (for example, diagnosis). As another example, with respect to FIG. 5, loading a historical dataset can include the negotiation engine 508 obtaining a historical dataset from the third party 510A and storing the historical dataset as part of the negotiation engine 508 or in storage accessibly by the negotiation engine 508.

The process 2300 optionally includes accessing a historical dataset stored on a computing system (2302B). For example, with respect to FIGS. 1A-1B, accessing a historical dataset can include the third-party computing system 108 accessing a historical dataset from the medical data 128. In more detail, the historical dataset can include research data obtained from a university or a government website related to a particular therapy or condition (for example, diagnosis). As another example, with respect to FIG. 5, accessing a historical dataset can include the negotiation engine 508 accessing a historical dataset stored on the negotiation engine 508 or in storage accessibly by the negotiation engine 508.

As an example, the historical dataset can be a historical data set can be a dataset for persons with a particular condition (for example, diagnosis) or having been treated with a particular therapy and can meet one or more criteria. The one or more criteria can include one or more of the following: includes data from a particular region, includes data from at least a minimum number of persons or patients, includes data for a particular dosage of a pharmaceutical drug, includes data for a particular number of cycles/packs/doses of a pharmaceutical drug per person for treating a particular condition, includes data collected that is no older than a particular time from the current time, etc.

The process 2300 includes populating an agreement on the computing system with details (2304). As an example, the agreement can be an agreement for the distribution of a therapy. As another example, the agreement can be an outcomes-based agreement (for example, outcomes-based agreement for the distribution of a particular therapy).

As an example, these details can include defining pharmaceutical therapy distribution constraints for the new scenario, assigning values to parameters for the new scenario, or a combination of the two. As another example, these details can include one or more of the following: one or more pharmaceutical drugs, a dosage of one or more pharmaceutical drugs, a condition (for example, diagnosis) to be treated, a region for distribution of a therapy, an agreement duration or duration range, a preferred start date for the agreement, a number of rounds of negotiation with one or more suppliers of the therapy, a number of patients per year or per duration of the contract, a number of therapy cycles required per patient, a price per cycle/pack/dose of therapy, a price per patient, rebate amounts, or the like. However, providing details can include assigning values to various other parameters, including pricing parameters.

In some implementations, populating the agreement with details includes structuring the agreement on the computing system.

The process 2300 includes back testing the structured and populated agreement against the historical data set (2306). As an example, back testing a populated agreement includes using an analytical tool to measure potential performance of the agreement (for example, budget impact performance, patient access performance, etc.). In more detail, back testing involves testing the populated against historical data to show what would have happened if an agreement had been performed at a previous point in time (for example, the previous three years if the agreement has a duration of three years, the previous year if the agreement has a duration of one year, etc.). This information can be used to inform possible future agreements and evaluate the effectiveness of an existing agreement.

As an example, with respect to FIGS. 1A-1B, the third-party computing system 108 back tests the structured and populated agreement against the historical data set.

As an example, with respect to FIG. 5, the negotiation engine 508 back tests the structured and populated agreement against the historical data set.

The process 2300 includes calculating past performance results based on the back test (2308). For example, the past performance results can indicate that the agreement would have been successful for the supplier and distributor (for example, profitable to both), successful for the supplier but not the distributor (for example, profitable for the supplier but not profitable or beyond a threshold risk of not being profitable for the distributor), or not successful for both the supplier and distributor. The past performance results can indicate that an agreement would not be successful for a distributor if, for example, it indicates that there are less anticipated patients than a number of patients considered by the agreement (for example, which may lead to the minimum number of therapy packs/cycles/doses to be purchased being greater than the number of therapy packs/cycles/doses that would have been used per the past performance), if the cost of the agreement would be greater than a budget for the agreement, if the agreement would not be profitable, etc.

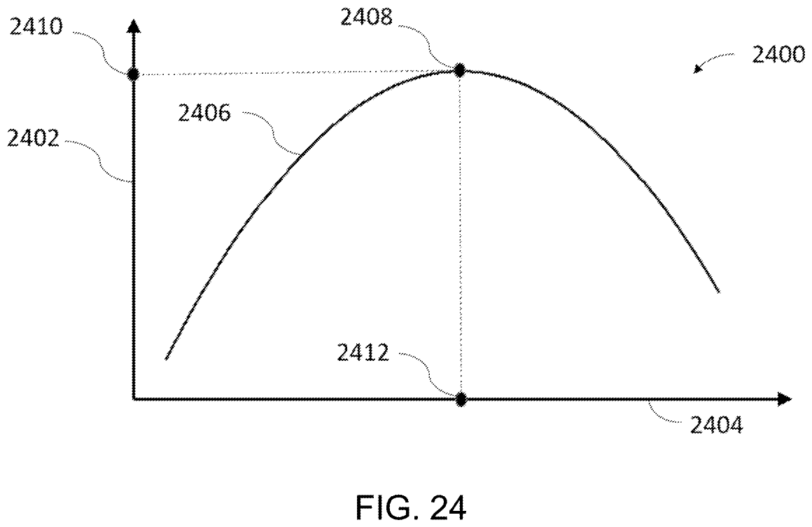

FIG. 24 depicts an optimization 2400 of a performance metric (for example the value of a quantity of a pharmaceutical therapy distributed during a specified period) with respect to the value 2404 of parameter in a pharmaceutical therapy distribution constraint (for example the value of a cap on the cost of the pharmaceutical therapy in a particular country), resulting in the performance curve 2406. The value of the performance metric increases with increasing values of the parameter until the value of the parameter reaches 2412, at which point the value of the performance metric reaches a maximum 2410, and after which the performance metric declines. In an example in which the performance metric is a quantity of a pharmaceutical therapy distributed during a specified period and the parameter is a price cap for the pharmaceutical therapy, above maximum 2408 the local supply of the pharmaceutical therapy diminishes (for example due to hoarding) causing the total quantity of pharmaceutical therapy distributed during the specified period to decline even though the price cap is lower. Therefore, the optimal point on the performance curve is point 2408 where the parameter value 2412 maximizes the performance metric (at a value 2410).

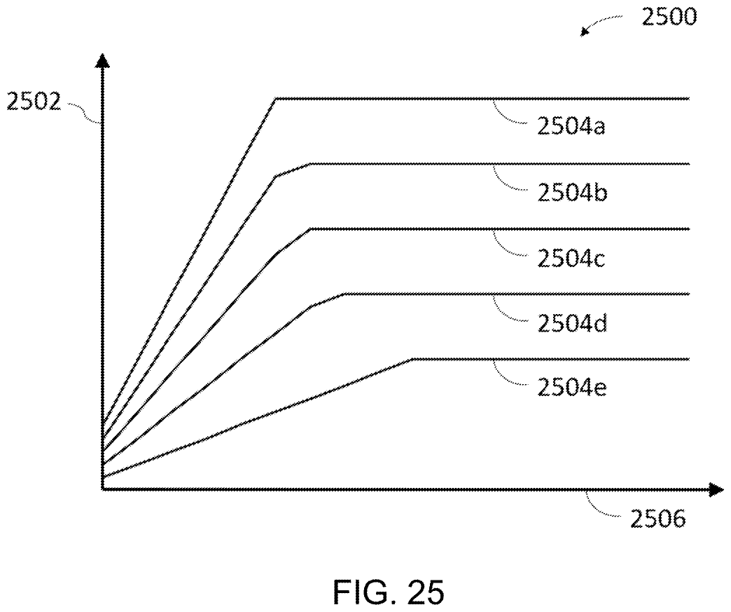

FIG. 25 depicts a series 2500 of ramp scenarios 2502a-e generated with respect to a parameter of a pharmaceutical therapy distribution constraint (the value of which is measured by axis 2504) as a function of time as measured by axis 2506. In each of ramp scenarios 2502a-e the value of the parameter increases at a different rate as a function of time up to a maximum value, after which the value of the parameter is fixed for a remaining period of time. For example, the parameter might be the number of new diagnoses of a condition per year for a condition that can be treated by a pharmaceutical therapy.

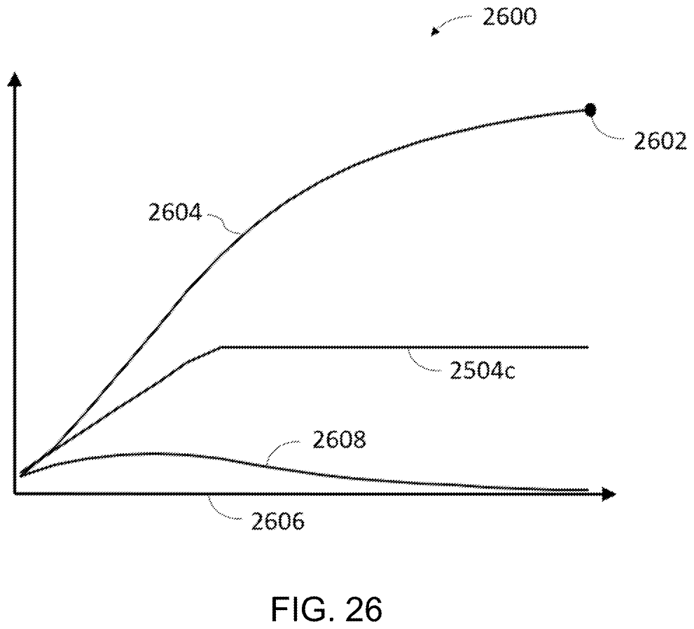

FIG. 26 depicts components 2600 of a calculation of a value 2602 of a performance metric with respect to scenario 2502c (see description of FIG. 25). For example, if the parameter is the number of new diagnoses of a condition per year for a condition that can be treated by a pharmaceutical therapy and the performance metric is the present discounted value of the distributed quantity of pharmaceutical therapy over time as measured by the time axis 2506, the present value of future distributions of the pharmaceutical therapy as they are distributed is shown by curve 2608 and the cumulative discounted value is shown by curve 2604, leading to a discounted value for future distributions of the pharmaceutical therapy up to a specified point of time in the future.

The subject matter described herein can be implemented in digital electronic circuitry, or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (for example, in a machine readable storage device), or embodied in a propagated signal, for execution by, or to control the operation of, data processing apparatus (for example, a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (for example, files that store one or more modules, sub programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, for example, an FPGA (field programmable gate array) or an ASIC (application specific integrated circuit).

Processors suitable for the execution of a computer program include, by way of example, both general and special purpose microprocessors, and any one or more processor of any kind of digital computer. Generally, a processor will receive instructions and data from a read only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, for example, magnetic, magneto optical disks, or optical disks. Information carriers suitable for embodying computer program instructions and data include all forms of nonvolatile memory, including by way of example semiconductor memory devices, (for example, EPROM, EEPROM, and flash memory devices); magnetic disks, (for example, internal hard disks or removable disks); magneto optical disks; and optical disks (for example, CD and DVD disks). The processor and the memory can be supplemented by, or incorporated in, special purpose logic circuitry.

To provide for interaction with a user, the subject matter described herein can be implemented on a computer having a display device, for example, a CRT (cathode ray tube) or LCD (liquid crystal display) monitor, for displaying information to the user and a keyboard and a pointing device, (for example, a mouse or a trackball), by which the user can provide input to the computer. Other kinds of devices can be used to provide for interaction with a user as well. For example, feedback provided to the user can be any form of sensory feedback, (for example, visual feedback, auditory feedback, or tactile feedback), and input from the user can be received in any form, including acoustic, speech, or tactile input.

The subject matter described herein can be implemented in a computing system that includes a back end component (for example, a data server), a middleware component (for example, an application server), or a front end component (for example, a client computer having a graphical user interface or a web browser through which a user can interact with an implementation of the subject matter described herein), or any combination of such back end, middleware, and front end components. The components of the system can be interconnected by any form or medium of digital data communication, for example, a communication network. Examples of communication networks include a local-area network (“LAN”) and a wide area network (“WAN”), for example, the Internet.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Particular embodiments of the invention have been described. Other embodiments are within the scope of the following claims. For example, the steps recited in the claims can be performed in a different order and still achieve desirable results. As one example, the processes depicted in the accompanying figures do not necessarily require the particular order shown, or sequential order, to achieve desirable results. In some cases, multitasking and parallel performance or processing may be advantageous.

### EMBODIMENTS

The following list provides embodiments of the invention and forms part of the description. These embodiments can be combined in any compatible combination beyond those expressly stated. The embodiments can also be combined with any compatible features described herein:
1. A computer-implemented method for distributing a pharmaceutical therapy to at least one patient, comprising:
a. configuring a computer-executable pharmaceutical distribution model that comprises a plurality of distribution constraints;
b. calculating a time-varying behavior of at least one parameter of the computer-executable pharmaceutical distribution model to obtain computational results;
c. quantifying a value of a first performance metric for the computational results;
d. adjusting the plurality of distribution constraints based at least on the computational results to form a computer-executable adjusted pharmaceutical distribution model that comprises an adjusted plurality of distribution constraints;
e. further calculating a time-varying behavior of at least one further parameter of the computer-executable adjusted pharmaceutical distribution model to obtain adjusted computational results;
f. further quantifying a value of a second performance metric for the adjusted simulation results; and
g. making the computer-executable adjusted pharmaceutical distribution model accessible to a third-party computing device.
2. The method of embodiment 1, further comprising receiving user input that is used in the configuring.
3 The method of any one of embodiments 1-2, further comprising providing user access to a sandbox environment for guiding one or more of the configuring, the adjusting, and the making.
4. The method of embodiment 3, wherein the user access is provided by one or more of a website interface, an application programming interface, and an app.
5. The method of any one of embodiments 1-4, wherein the making comprises providing a URL for onboarding a user, the URL providing restricted access to the pharmaceutical distribution model.
6. The method of any one of embodiments 1-5, wherein the computational results comprises results for a plurality of time-dependent scenarios that are a function of the parameter.
7. The method of embodiment 6, wherein the value of the first performance metric comprises an average of results determined from the plurality of time-dependent scenarios.
8. The method of any one of embodiments 6-7, wherein the plurality of time-dependent scenarios are generated at least in part using a random number generator.
9 The method of any one of embodiments 1-8, wherein the computational results comprise a solution to a mathematical program that optimizes the parameter so as to maximize or minimize the value of the first performance metric subject to the distribution constraints.
10. The method of embodiment 9, wherein mathematical program is a mixed-integer linear program.
11. The method of embodiment 9, wherein the mathematical program is a mixed-integer nonlinear program.
12. The method of any one of embodiments 1-11, wherein the computational results comprise a sensitivity analysis with respect to the parameter.
13. The method of any one of embodiments 1-12, wherein the plurality of distribution constraints do not comprise any proprietary data.
14. The method of any one of embodiments 1-13, wherein a distribution constraint of the plurality of distribution constraints comprises a statistical model derived from third-party proprietary data.
15. The method of any one of embodiments 1-14, wherein the adjusted pharmaceutical distribution model is formed at least in part by restricting a portion of the pharmaceutical distribution model.
16. The method of any one of embodiments 1-15, wherein the adjusting is further based at least on the value of the first performance metric.
17. The method of any one of embodiments 1-16, wherein the making further comprises making the value of the second performance metric accessible to the third-party computing device, and the value of the second performance metric is greater than the value of the first performance metric.
18. The method of any one of embodiments 1-17, wherein the adjusted pharmaceutical distribution model has lower dimensionality than the pharmaceutical distribution model.
19. The method of any one of embodiments 1-18, wherein the method is implemented in a platform hosted on a website.
20. The method of any one of embodiments 1-19, the method further comprising facilitating an agreement between multiple parties for the distributing a pharmaceutical therapy.
21. The method of embodiment 20, wherein facilitating the agreement between multiple parties for the distributing a pharmaceutical therapy comprises distributing a pharmaceutical therapy to patients.
22. The method of any one of embodiments 1-21, the method further comprising enabling a first party of the multiple parties and a second party of the multiple parties to identify common areas of agreement with respect to the variables and/or terms of the proposed distribution agreement.
23. The method of embodiment 22, wherein the common areas of agreement comprise win-win provisions.
24. The method of any one of embodiments 1-23, the method further comprising enabling a party of (i) the multiple parties or (ii) each of the parties to perform a computer simulation with respect to variables and/or terms of a proposed distribution agreement.
25. The method of embodiment 24, wherein enabling the party to perform the computer simulation comprises using software implemented using a programming language such as JAVA.
26. The method of any one of the embodiments 24-25, wherein enabling the party to perform the computer simulation comprises enabling the party to perform the computer simulation prior to negotiation of or during negotiation of the proposed distribution agreement.
27. The method of any one of the embodiments 24-26, wherein the computer simulation tracks targets of the party with respect to the proposed distribution agreement over a prospective time horizon.
28. The method of embodiment 27, wherein the targets comprise one or more goals such as distribution goals for the pharmaceutical therapy.
29. The method of any one of embodiments 1-28, the method further comprising enabling a party of (i) the multiple parties or (ii) each of the parties to perform sensitivity analysis with respect to variables and/or terms of a proposed distribution agreement.
30. The method of embodiment 29, wherein enabling the party to perform sensitivity analysis comprises enabling the party to perform the sensitivity analysis prior to negotiation of or during negotiation of the proposed distribution agreement.
31. The method of any one of embodiments 1-30, the method further comprising reducing the time to close the proposed distribution agreement by sharing simulation results.
32. The method of any one of embodiments 1-31, the method further comprising reducing the time to close the proposed distribution agreement by reducing redundant analysis.
33 The method of any one of embodiments 1-32, the method further comprising reducing the time to close the proposed distribution agreement by consolidating deal information on a shared or at least partially shared analytical platform.
34. The method of any one of embodiments 1-33, the method further comprising enabling a first party of the multiple parties to share the results of the simulation with a second party of the multiple parties.
35. The method of any one of embodiments 1-34, the method further comprising reducing the time to close the proposed distribution agreement by defining one or more simulation scenarios of the proposed distribution agreement as a basis for negotiation of terms of the proposed distribution agreement.
36. The method of any one of embodiments 1-35, the method further comprising enabling an intermediary between the multiple parties to share the results of the simulation with a party of the multiple parties.
37. The method of embodiment 36, wherein the intermediary is an arranger of the proposed distribution agreement or a provider of a platform that implements the method.
38. The method of any one embodiment 1-37, the method further comprising inviting and/or onboarding, by a first party of the multiple parties, a second party of the multiple parties to access variables and/or terms and or simulation results associate with the method for the proposed distribution agreement
39. The method of embodiment 38, wherein inviting and/or onboarding a second party comprises inviting and/or onboarding a second party via an email containing a URL.
40. The method of any one of embodiments 38-39, wherein the simulation results comprise simulation results for a scenario.
41. The method of any one of embodiments 38-40, wherein a URL received by the second party provides restricted computer simulation functionality compared to computer simulation functionality enjoyed by the first party.
42. The method of any one of embodiments 38-41, wherein (i) the first party or (ii) an intermediary between the first party and the second party controls the computer simulation functionality that is available to the second party.
43. The method of anyone of embodiments 38-41, wherein the second party is granted limited access to the simulation results for the scenario.
44. The method of embodiment 43, wherein the limited access is read-only access.
45. The method of any one of embodiments 1-44, the method further comprising enabling a party of the multiple parties to wargame the proposed distribution agreement,
46. The method of any one of embodiments 1-45, the method further comprising enabling a first party of the multiple parties to translate terms of the distribution agreement into related terms for a second party of the multiple parties.
47. The method of embodiment 46, wherein enabling the first party of the multiple parties to translate terms of the distribution agreement into related terms for the second party comprises translating the first party's production budget for the pharmaceutical therapy into a second party's consumption budget and/or revenue or expense stream.
48. The method of any one of embodiments 1-47, wherein the computer simulation is implemented in computer executable software that generates a string for display to a party of the multiple parties.
49. The method of embodiment 48, wherein the string is associated with a field that is displayed via a computer-interface to the party.
50. The method of embodiment 49, wherein the string includes distribution agreement information in a configurable format.
51. The method of embodiment 50, wherein the distribution agreement information in a configurable format comprises information displayed on a per-contract basis, consolidated basis.
52. The method of any one of embodiments 1-51, the method further comprising enabling creation of a non-transitory computer readable media containing deal terms for one or more pharmaceutical therapy distribution agreements.
53. The method of any one of embodiments 1-53, the method further comprising enabling creation of a non-transitory computer readable media containing simulation information for one or more pharmaceutical therapy distribution agreements.
54 The method of embodiment 53, wherein the simulation information comprises simulation results and/or predictions.
55. The method of any one of embodiments 1-54, the method further comprising enabling creation of a non-transitory computer readable media containing historical tracking information for one or more pharmaceutical therapy distribution agreements that has been implemented.
56. The method of embodiment 55, wherein the historical tracking information comprises a time series of distribution amounts for the pharmaceutical therapy.
57. The method of any one of embodiments 1-56, the method further comprising enabling creation of a non-transitory computer readable media containing a comparison of historical tracking information and simulation information for one or more pharmaceutical therapy distribution agreements that has been implemented.
58 The method of any one of embodiments 1-57, the method further comprising enabling post-negotiation analysis of a pharmaceutical distribution agreement.
59. The method of any one of embodiments 1-58, the method further comprising enabling back-testing of a proposed pharmaceutical distribution agreement.
60. The method of any one of embodiments 1-59, the method further comprising back-testing a proposed pharmaceutical distribution agreement.
61. The method of any one of embodiments 1-60, the method further comprising performing post-negotiation analysis of a pharmaceutical distribution agreement.
62. The method of any one of embodiments 1-61, wherein the pharmaceutical therapy comprises a diagnosis.
63. The method of any one of embodiments 1-62, wherein the pharmaceutical therapy comprises a code.
64. The method of embodiment 63, wherein the code is a diagnosis code, a regimen code, or a billing code.
65. The method of any one of embodiments 1-64, wherein the pharmaceutical therapy is associated with a therapy type.
66. The method of embodiment 65, wherein the therapy type is monotherapy or a combination therapy.
67. The method of any one of embodiments 1-66, wherein the pharmaceutical therapy is associated with a market.
68. The method of any one of embodiments 1-67, wherein the pharmaceutical therapy comprises a treatment to treat a condition.
69. The method of embodiment 68, wherein the treatment comprises administration of a drug.
70. The method of any one of embodiments 68-69, wherein the condition is a disease.
71. The method of any one of embodiments 1-70, wherein the pharmaceutical therapy comprises a treatment to prevent a condition.
72. The method of any one of embodiments 1-71, wherein the pharmaceutical therapy comprises a treatment regimen.
73 The method of embodiment 72, wherein the treatment regimen comprises a dosing schedule for a drug.
74. The method of embodiment 73, wherein the dosing schedule comprises a start cycle, a cycle length, an end cycle, and a dosing for all days in each of the foregoing cycles.
75. The method of any one of embodiments 73-74, wherein the dosing schedule comprises a number of administrations of a product per day and a dosage amount per administration.
76. The method of embodiment 75, wherein the product comprises a pharmaceutical drug.
77. The method of any one of embodiments 74-76, wherein the dosing schedule comprises a dosing for all days.
78. The method of any one of embodiments 1-77, wherein the pharmaceutical therapy comprises a new drug therapy.
79. The method of any one of embodiments 1-78, wherein the pharmaceutical therapy has an associated product pack.
80. The method of embodiment 79, wherein the product pack is based on an average dosage of a pharmaceutical compound to be administered to a patient within a pre-determined period of time.
81. The method of any one of embodiments 1-80, wherein the configuring comprises receiving input data from a user.
82. The method of any one of embodiments 1-81, wherein the computer-executable pharmaceutical distribution model is accessible to the user via a website interface.
83. The method of any one of embodiments 1-82, wherein the computer-executable pharmaceutical distribution model is accessible to the user via a downloadable app.
84. The method of any one of embodiments 1-83, wherein the computer-executable pharmaceutical distribution model is configured to execute remotely from the user.
85. The method of any one of embodiments 1-84, wherein the computer-executable pharmaceutical distribution model configured to execute on a computer device under the control of the user.
86. The method of any one of embodiments 1-85, wherein the constraints comprise equalities.
87. The method of any one of embodiments 1-86, wherein the plurality of distribution constraints define a supply amount as a function, at least in part, of an effectiveness of the pharmaceutical therapy.
88. The method of any one of embodiments 1-87, wherein the plurality of distribution constraints define a supply amount as a function, at least in part, of a size of a patient population.
89. The method of embodiment 88, wherein the size of the patient population is a time-dependent size of the patient population.
90. The method of any one of embodiments 1-89, wherein the plurality of distribution constraints define a supply amount as a function, at least in part, of a number of treatments per patient.
91. The method of embodiment 90, wherein the number of treatments per patient is a number of treatments per patient over an expected course of the pharmaceutical therapy, a number of treatments per patient during a given time, or a number of treatment cycles per year.
92. The method of any one of embodiments 1-91, wherein the plurality of distribution constraints define a supply amount as a function, at least in part, of patient adherence to the pharmaceutical therapy.
93. The method of embodiment 92, wherein the patient adherence is the percentage of patients in a patient population that complete a prescribed number of treatment cycles of the pharmaceutical treatment.
94 The method of any one of embodiments 1-93, wherein the plurality of distribution constraints comprise constraints on one or more funding amounts for the distribution of the pharmaceutical therapy.
95. The method of embodiment 94, wherein the constraints on one or more funding amounts comprise an upfront payment.
96. The method of embodiment 95, wherein the constraints on one or more funding amounts comprise a further payment that is contingent upon a patient response to the pharmaceutical therapy.
97. The method of embodiment 96, wherein the patient response to the pharmaceutical therapy is a positive per-patient response or a measure of an aggregate response to the pharmaceutical therapy by a plurality of patients.
98. The method of any one of embodiments 96-97, wherein the further payment is larger than the upfront payment.
99 The method of any one of embodiments 94-98, wherein constraints on one or more funding amounts comprise a rebate that is contingent upon a patient response to the pharmaceutical therapy.
100. The method of embodiment 99, wherein the patient response to the pharmaceutical therapy is a negative per-patient response or a measure of an aggregate response to the pharmaceutical therapy by a plurality of patients.
101. The method of any one of embodiments 99-100, wherein the rebate is smaller than the upfront payment.
102. The method of any one of embodiments 94-101, wherein the constraints on one or more funding amounts comprise a discount to a funding amount of the one or more funding amounts as a function of a total number of patients that receive the pharmaceutical therapy.
103. The method of embodiment 102, wherein the discount is applied on a per-patient basis.
104. The method of any one of embodiments 102-103, wherein the discount requires a specified level of patient adherence to the pharmaceutical therapy.
105. The method of any one of embodiments 102-104, wherein the discount is tiered for different patients in a tier.
106. The method of any one of embodiments 94-105, wherein the constraints on one or more funding amounts comprise a discount to a funding amount of the one or more funding amounts as a function of a previous funding provided during a prior specified time period.
107. The method of embodiment 106, wherein the discount to the funding amount comprises a discount applied to future fundings during a calendar year when previous fundings exceeds a predetermined amount within the calendar year.
108. The method of any one of embodiments 94-107, wherein the constraints on one or more funding amounts comprise a cap on funding during a specified time period.
109. The method of embodiment 108, wherein the cap on funding is a cap on funding expenditures during a calendar year on a per-patient basis.
110. The method of embodiment 108, wherein the cap on funding is a cap on total funding during a specific time period.
111. The method of embodiment 108, wherein the cap on funding is a limit on payments to payments incurred during a specified portion of a time period.
112. The method of embodiment 111, wherein the limit is a limit on payments for receipt of the pharmaceutical therapy to those payments incurred during the first 10 months of a calendar year.
113. The method of embodiment 108, wherein the cap on funding is applied on a per patient basis.
114. The method of embodiment 108, wherein the cap on funding is an aggregate cap for a group of patients.
115. The method of embodiment 114, wherein the group of patients is a population of patients in a geographic region.
116. The method of any one of embodiments 114-115, wherein the constraints on one or more funding amounts further comprises a rebate that is triggered once the one or more funding amounts reaches the aggregate cap.
117. The method of embodiment 116, wherein the rebate is effective for a specified incremental amount above the aggregate cap.
118. The method of any one of embodiments 94-117, wherein the constraints on one or more funding amounts comprise an periodic funding amount for a specified number of periods.
119. The method of embodiment 118, wherein the periodic funding amount is a specified funding amount for each of a set number of years.
120. The method of any one of embodiments 118-119, wherein the periodic funding amount for the specified number of periods funds treatment of an unlimited number of patients with the pharmaceutical therapy during the specified number of periods.
121. The method of any one of embodiments 94-120, wherein the constraints on one or more funding amounts comprise a specified number of free treatments with the pharmaceutical therapy.
122. The method of embodiment 121, wherein the specified number of free treatments is an initial number of free treatments or an additional number of free treatments following funding of an initial number of treatments.
123. The method of any one of embodiments 121-122, wherein the number of free treatments is determined from a number of funded treatments.
124. The method of any one of embodiments 94-123, wherein the constraints on one or more funding amounts comprise a deferral of funding for a set of treatments with the pharmaceutical therapy until the pharmaceutical therapy receives regulatory approval.
125. The method of embodiment 124, wherein the funding is at a discounted rate relative to a price of the pharmaceutical therapy that is established based on approval of the pharmaceutical therapy.
126. The method of any one of embodiments 1-125, wherein the plurality of distribution constraints comprises inequalities.
127. The method of embodiment 126, wherein the plurality of distribution constraints comprise a bounding value for a supply amount of the pharmaceutical therapy.
128. The method of embodiment 127, wherein the bounding value is a maximum value or a minimum value for a supply amount of the pharmaceutical therapy.
129. The method of any one of embodiments 127-128, wherein the bounding value for the supply amount is computed from an incremental change to a supply amount of the pharmaceutical therapy relative to an initially specified supply mount of the pharmaceutical therapy.
130. The method of embodiment 129, wherein the incremental change is a minimum increase, a minimum reduction, a maximum increase, or a maximum reduction.
131. The method of any one of embodiments 127-130, wherein the bounding value is a function, at least in part, of an effectiveness of the pharmaceutical therapy.
132. The method of embodiment 131, wherein the effectiveness of the pharmaceutical therapy is a a known effectiveness, an average effectiveness, a predicted effectiveness, a simulated effectiveness, or an optimized effectiveness; or wherein the bounding value is computed from an incremental change to the supply amount that is triggered by a threshold level of deviation of the effectiveness from an initial expectation of the effectiveness of the pharmaceutical therapy.
133. The method of embodiment 132, wherein the incremental change is a minimum increase, a minimum reduction, a maximum increase, or a maximum reduction.
134. The method of any one of embodiments 127-133, wherein the bounding value is a function, at least in part, of a realized size of a patient population for the pharmaceutical therapy.
135. The method of embodiment 134, wherein the bounding value is computed from an incremental change to the supply amount that is triggered by a threshold level of deviation of the realized size from an initial expectation of the size of the patient population.
136. The method of embodiment 135, wherein the incremental change is a minimum increase, a minimum reduction, a maximum increase, or a maximum reduction.
137. The method of any one of embodiments 127-136, wherein the bounding value is a function, at least in part, of a number of treatments per patient.
138. The method of embodiment 137, wherein the bounding value is computed from an incremental change to the supply amount that is triggered by a threshold level of deviation of the realized number of treatments per patient from an initial expectation of the number of treatments per patient.
139. The method of embodiment 138, wherein the incremental change is a minimum increase, a minimum reduction, a maximum increase, or a maximum reduction.
140. The method of any one of embodiments 127-139, wherein the bounding value is a function, at least in part, of a number of discontinuation recommendations for the pharmaceutical therapy.
141. The method of embodiment 140, wherein the bounding value is computed from an incremental change to the supply amount that is triggered by a threshold level of deviation of the number of discontinuation recommendations from an initial expectation of the effectiveness of the number of discontinuation recommendations.
142. The method of embodiment 141, wherein the incremental change is a minimum increase, a minimum reduction, a maximum increase, or a maximum reduction.
143. The method of any one of embodiments 127-141, wherein the bounding value is a function, at least in part, of an available supply of the pharmaceutical therapy.
144. The method of embodiment 143, wherein the bounding value is computed from an incremental change to the supply amount that is triggered by a threshold level of deviation of the available supply from an initial expectation of the available supply.
145. The method of embodiment 144, wherein the incremental change is a minimum increase, a minimum reduction, a maximum increase, or a maximum reduction.
146. The method of any one of embodiments 127-145, wherein the bounding value is a function, at least in part, of a regulatory status of the pharmaceutical therapy.
147. The method of embodiment 146, wherein the bounding value is computed from an incremental change to the regulatory status of the pharmaceutical therapy that is triggered by a threshold level of deviation of the regulatory status from an initial expectation of the regulatory status
148. The method of embodiment 147, wherein the incremental change is a minimum increase, a minimum reduction, a maximum increase, or a maximum reduction.
149. The method of any one of embodiments 127-148, wherein the bounding value is a function, at least in part, of a geographical demand for the pharmaceutical therapy.
150. The method of embodiment 149, wherein the geographical demand is a demand for the pharmaceutical therapy by a region, country, state, or county.
151. The method of any one of embodiments 149-150, wherein the bounding value is computed from an incremental change to the geographical demand of the pharmaceutical therapy that is triggered by a threshold level of deviation of the geographical demand from an initial expectation of the geographical demand.
152. The method of embodiment 151, wherein the incremental change is a minimum increase, a minimum reduction, a maximum increase, or a maximum reduction.
153. The method of any one of embodiments 127-152, wherein the bounding value is a function, at least in part, of a legal constraint on the pharmaceutical therapy.
154. The method of embodiment 153, wherein the legal constraint is an export ban, import ban, or tariff.
155. The method of any one of embodiments 153-154, wherein the bounding value is computed from an incremental change to the legal constraint that is triggered by a threshold level of deviation of the legal constraint from an initial expectation of the legal constraint or lack thereof.
156. The method of embodiment 155, wherein the incremental change is a minimum increase, a minimum reduction, a maximum increase, or a maximum reduction.
157. The method of any one of embodiments 127-156, wherein the bounding value is a function, at least in part, of a governmental factor.
158. The method of embodiment 157, wherein the governmental factor is a government subsidy.
159. The method of any one of embodiments 157-158, wherein the bounding value is computed based on a change to a governmental factor
160. The method of embodiment 159, wherein the governmental factor is (i) an allowance or withdrawal of a government subsidy for the pharmaceutical therapy or (ii) a government reimbursement rate for the pharmaceutical therapy.
161. The method of any one of embodiments 127-160, wherein the bounding value is a function, at least in part, of a reimbursement rate for the pharmaceutical therapy.
162. The method of embodiment 161, wherein the bounding value is computed from a change to the reimbursement rate of the pharmaceutical therapy.
163. The method of embodiment 162, wherein the incremental change is a minimum increase, a minimum reduction, a maximum increase, or a maximum reduction.
164. The method of any one of embodiments 1-164, wherein a constraint of the plural distribution constraints comprises a discount that depends on the total number of the one or more patients to which the pharmaceutical therapy is distributed.
165. The method of any one of embodiments 1-164, wherein the configuring comprises setting values for a plurality of parameters associated with the distribution constraints.
166. The method of embodiment 165, wherein the plurality of parameters comprises coefficients of variables in the distribution constraints.
167. The method of any one of the embodiments 165-166, wherein the plurality of parameters comprise binary variables that multiplies at least one distribution constraint of the plurality of distribution constraints.
168. The method of any one of the embodiments 165-167, wherein a parameter of the plurality of parameters corresponds to a variable that is associated with the plurality of distribution constraints.
169. The method of embodiment 168, wherein the variable is a continuous variable.
170. The method of any one of the embodiments 168-169, wherein the variable is a discrete variable.
171. The method of any one of the embodiments 168-170, wherein the plurality of distribution constraints comprise distribution constraint that defines a bound on the variable.
172. The method of embodiment 171, wherein the bound on the variable is an upper bound and/or a lower bound.
173. The method of any one of the embodiments 1-172, wherein the distribution constraints comprise a plurality of archetypal constraints.
174. The method of embodiment 1-173, wherein a first archetypal constraint of the plurality of archetypal constraints is mutually exclusive to a second archetypal constraint of the plurality of archetypal constraints.
175. The method of any one of the embodiments 1-174, wherein the configuring comprises generating source code for the computer-executable pharmaceutical distribution model.
176. The method of any one of the embodiments 1-175, wherein the computer-executable pharmaceutical distribution model is implemented on a server.
177. The method of any one of the embodiments 1-176, wherein the computer-executable model is configured to be executed by an analytical engine.
178. The method of any one of the embodiments 1-177, wherein the configuring is guided at least in part by a distributor of the pharmaceutical therapy operating a computing device.
179. The method of any one of the embodiments 1-178, wherein the configuring is guided at least in part by a producer of the pharmaceutical therapy operating a computing device.
180. The method of any one of the embodiments 1-179, wherein the configuring is guided at least in part by an intermediary operating on behalf of a group of patients operating a computing device.
181. The method of any one of the embodiments 1-180, wherein the configuring is guided at least in part by a patient operating a computing device.
182. The method of any one of the embodiments 1-181, wherein the configuring is guided at least in part by an intermediary between a first distributor of the pharmaceutical therapy and a second distributor of the pharmaceutical therapy.
183. The method of embodiment 182, wherein the first distributor of the pharmaceutical therapy is a supplier of at least one component of the pharmaceutical treatment.
184. The method of embodiment 183, wherein the supplier is manufacture of the pharmaceutical therapy.
185. The method of any one of embodiments 183-184, wherein the at least one component comprises a pharmaceutical drug or an active ingredient in a pharmaceutical drug.
186. The method of any one of embodiments 1-185, wherein the configuring comprises setting a value corresponding to a parameter of a plurality of parameters that are associated with the plurality of distribution constraints.
187. The method of embodiment 186, wherein the value is generated using a computer-implemented statistical model.
188. The method of any one of embodiments 186-187, wherein the value is one of a set of values corresponding to the parameter at different instances in time.
189. The method of any one of embodiments 186-188, wherein the value of one of a set of values corresponding to multiple scenarios for the parameter.
190. The method of any one of embodiments 186-189, wherein the plurality of parameters comprises coefficients of variables in the plurality of distribution constraints.
191. The method of any one of embodiments 186-190, wherein the plurality of parameters comprises variables in the plurality of distribution constraints.
192. The method of any one of embodiments 186-191, wherein the plurality of parameters comprise a binary variable that multiplies a distribution constraint of the plurality of distribution constraints.
193. The method of any one of embodiments 1-192, wherein the method further comprises generating a plurality of computer-executable pharmaceutical distribution models.
194. The method of embodiment 193, wherein the method further comprises computing a ranking of a first model of the plurality of computer-executable pharmaceutical distribution models relative to a second model of the plurality of computer-executable pharmaceutical distribution models.
195. The method of embodiment 194, wherein the ranking is based on relevance.
196. The method of embodiment 195, wherein relevance is based at least in part on a specified drug dosage.
197. The method of any one of embodiments 195-196, wherein relevance is based at least in part on a duration of the pharmaceutical therapy.
198. The method of any one of embodiments 195-197, wherein relevance is based at least in part on a specified region, country, state, county, or other geographic subdivision.
199. The method of any one of the embodiment 194-198, wherein the ranking is displayed on a user interface.
200. The method of any one of embodiments 1-199, wherein calculating the time-varying behavior comprises performing calculations to enforce a distribution constraint of the plurality of distribution constraints.
201. The method of embodiment 200, wherein the calculations to enforce the distribution constraint comprise determining a value for a variable that allows the distribution constraint to be enforced.
202. The method of any one of embodiments 1-201, wherein calculating the time-varying behavior comprises performing calculations to determine a time series of values for a variable present in a distribution constraint of the plurality of distribution constraints.
203. The method of any one of embodiments 1-202, wherein the calculating the time-varying behavior comprises a dynamic simulation.
204. The method of any one of embodiments 1-203, wherein the calculating the time-varying behavior comprises calculating the time-varying behavior of the at least one parameter for a scenario.
205. The method of any one of embodiments 1-204, wherein the calculating the time-varying behavior comprises calculating the time-varying behavior of the at least one parameter for a plurality of scenarios.
206. The method of any one of embodiments 1-205, wherein the calculating the time-varying behavior comprises a Monte Carlo simulation.
207. The method of any one of embodiments 1-206, wherein the calculating the time-varying behavior is performed during an optimization process.
208. The method of embodiment 207, wherein the optimization process comprises at least partially solving a linear program.
209. The method of any one of embodiments 207-208, wherein the optimization process comprises at least partially solving a nonlinear program.
210. The method of any one of embodiments 207-209, wherein the optimization process comprises at least partially solving an integer program.
211. The method of any one of embodiments 207-210, wherein the optimization process comprises at least partially solving an integer-linear program.
212. The method of any one of embodiments 207-211, wherein the optimization process comprises at least partially solving a mixed integer-nonlinear program.
213. The method of any one of embodiments 207-212, wherein the optimization process comprises simulated annealing.
214. The method of any one of embodiments 207-213, wherein the optimization process at least partially optimizes a parameter of the plurality of parameters.
215. The method of embodiment 214, wherein the parameter is a variable associated with the plurality of distribution constraints.
216. The method of any one of the embodiments 214-215, wherein the parameter is a coefficient of a variable that is associated with the plurality of distribution constraints.
217. The method of any one of the embodiments 214-216, wherein the parameter is a binary parameter that multiples another parameter that is associated with the plurality of distribution constraints.
218. The method of any one of the embodiments 1-217, wherein the time-varying behavior comprises a change in a distribution quantity of the pharmaceutical therapy.
219. The method of embodiment 218, wherein the change is an instantaneous change or a cumulative change.
220. The method of any one of the embodiments 1-219, wherein the time-varying behavior comprises a change in a patent adherence to the pharmaceutical therapy.
221. The method of any one of the embodiments 1-220, wherein the time-varying behavior comprises a change in a residual of a distribution constraint of the plurality of distribution constraints.
222. The method of any one of the embodiments 1-221, wherein the time-varying behavior comprises a change in a supply of the pharmaceutical treatment.
223. The method of any one of the embodiments 1-222, wherein the time-varying behavior comprises a change in a number of discontinuation recommendations for the pharmaceutical therapy.
224. The method of any one of the embodiments 1-223, wherein the time-varying behavior comprises a change in an available supply for the pharmaceutical therapy.
225. The method of any one of the embodiments 1-224, wherein the time-varying behavior comprises a change in a production rate for the pharmaceutical therapy.
226. The method of any one of the embodiments 1-225, wherein the time-varying behavior comprises a change in a regulatory status for the pharmaceutical therapy.
227. The method of embodiment 226, wherein the regulatory status is regulatory approval, regulatory pending, or regulatory rejection.
228. The method of any one of the embodiments 1-227, wherein the time-varying behavior comprises a change in a legal status for the pharmaceutical therapy.
229. The method of any one of the embodiments 1-228, wherein the time-varying behavior comprises a change in a geographic factor for the pharmaceutical therapy.
230. The method of embodiment 229, wherein the geographic factor is a geographic supply factor or a geographic demand factor.
231. The method of any one of the embodiments 1-230, wherein the time-varying behavior comprises a change in a governmental factor for the pharmaceutical therapy.
232. The method of embodiment 231, wherein the governmental factor is a government subsidy.
233. The method of any one of the embodiments 1-232, wherein the time-varying behavior comprises a change in a reimbursement rate (for example an insurance reimbursement rate for the pharmaceutical therapy.
234. The method of any one of the embodiments 1-233, wherein the computational results comprise a residual of a distribution constraint of the plurality of distribution constraints.
235. The method of any one of the embodiments 1-234, wherein the computational results comprise a selection of constraints from the plurality of distribution constraints.
236. The method of any one of the embodiments 1-235, wherein the computational results comprise residual values for a constraint of the plurality of distribution constraints.
237. The method of any one of the embodiments 1-236, wherein the computational results comprise a time series of values for a variable associated with the plurality of distribution constraints.
238. The method of any one of the embodiments 1-237, wherein the pharmaceutical distribution model comprises fixed coefficients.
239. The method of embodiment 238, wherein the fixed coefficients are obtained from a third-party data source.
240. The method of any one of the embodiments 238-239, wherein the method further comprises computing some or all of the fixed coefficients.
241. The method of any one of the embodiments 1-240, wherein the first performance metric comprises a number of patients treated using the pharmaceutical therapy during one or more specified time periods.
242. The method of any one of the embodiments 1-241, wherein the first performance metric comprises a size of a patient population that is eligible to receive the pharmaceutical therapy during one or more specified time periods or points in time.
243. The method of any one of the embodiments 1-242, wherein the first performance metric comprises a metric of patient adherence to the pharmaceutical therapy during one or more specified time periods or points in time.
244. The method of any one of the embodiments 1-243, wherein the first performance metric comprises a measure of funding associated with the distribution of the pharmaceutical therapy.
245. The method of any one of the embodiments 1-244, wherein the first performance metric comprises a measure of return associated with the distribution of the pharmaceutical therapy.
246. The method of embodiment 245, wherein the measure of return is total return, a margin, or a yield.
247. The method of any one of the embodiments 1-246, wherein the first performance metric comprises a measure of financial impact on patients associated with use of the pharmaceutical therapy.
248. The method of any one of the embodiments 1-247, wherein the first performance metric comprises a measure of the number of discontinuation recommendations for the pharmaceutical therapy.
249. The method of any one of the embodiments 1-248, wherein the first performance metric comprises a price for the pharmaceutical therapy.
250. The method of embodiment 249, wherein the price is a price-per-patient, a price-per-pack, or a price ratio of prices in different countries, states, counties, or other regions.
251. The method of any one of embodiments 249-250, the price is a future price, an average price over a specified time period, or a price in a specified geography.
252. The method of any one of embodiments 1-251, wherein the first performance metric comprises a measure of geographic breadth of distribution of the pharmaceutical therapy.
253. The method of any one of embodiments 1-252, wherein the quantifying comprises calculating an average of measurements of the first performance metric for a plurality of scenarios.
254. The method of embodiment 253, wherein the average is a simple average or a weighted average.
255. The method of any one of embodiments 1-253, wherein the quantifying comprises calculating a statistical measure of the first performance metric.
256. The method of embodiment 255, wherein the statistical measure of the first performance metric is a statistical measure of the first performance metric based on a plurality of scenarios.
257. The method of any one of embodiments 1-256, wherein the quantifying comprises calculating a weighted average of measurements of the first performance metric for a plurality of scenarios.
258. The method of embodiment 257, wherein the measurements of the first performance metric for the plurality of scenarios are weighted by eliminating a maximum measurement of the first performance metric and/or a minimum measurement of the first performance metric.
259. The method of any one of embodiments 1-258, wherein the adjusting is further based at least on the computational results.
260. The method of any one of embodiments 1-259, wherein the adjusting comprises replacing a parameter in the plurality of distribution constraints with a fixed value.
261. The method of embodiment 260, wherein the parameter is a coefficient or a variable.
262. The method of any one of embodiments 260-261, herein the fixed value is determined from an optimization with respect to the parameter.
263. The method of any one of embodiments 1-262, wherein the adjusting comprises removing one or more of the distribution constraints from the plurality of distribution constraints.
264. The method of any one of embodiments 1-263, wherein the adjusting comprises adding a new distribution constraint to the plurality of distribution constraints.
265. The method of any one of embodiments 1-264, wherein the at least one further parameter corresponds to the at least one parameter.
266. The method of any one of embodiments 1-265, wherein the at least one further parameter does not completely correspond to the at least one parameter.
267. The method of any one of embodiments 1-266, wherein the second performance metric corresponds to the first performance metric.
268. The method of embodiment 267, wherein the second performance metric is the same as the first performance metric.
269. The method of any one of embodiments 1-268, wherein the second performance metric does not correspond to the first performance metric.
270. The method of any one of embodiments 1-269, the method further comprising making the adjusted computational results available to the third-party computing device.
271. The method of any one of embodiments 1-269, the method further comprising making the value of the second performance metric available to the third-party computing device.
272. A computer-implemented method for backtesting a pharmaceutical therapy to at least one patient, comprising:
a. configuring a computer-executable pharmaceutical distribution model that comprises a plurality of distribution constraints;
b. assigning at least one defined scenario for (i) a parameter or (ii) a plurality of parameters of the computer-executable pharmaceutical distribution model;
c. simulating the computer-executable pharmaceutical distribution model based at least in part on the defined scenario to obtain computational results;
d. quantifying a value of a performance metric for the computational results;
e. defining a data set for the parameter;
f. further calculating the time-varying behavior of the at least one parameter based on the historical data set to obtain backtesting computational results;
g. further quantifying a value of the performance metric for the backtesting computational results; and
h. adjusting a distribution constraint of the plurality of distribution constraints based on a difference between the computational results and the backtesting computational results or making the backtesting computational results accessible to a remote (for example third party) computing device.
273. The method of embodiment 272, wherein the at least one parameter comprises a time-varying parameter.
274. The method of any one of embodiments 272-273, wherein the at least one parameter comprises a static parameter.
275. The method of any one of embodiments 272-274, wherein the data set is a historical data set.
276. The method of embodiment 275, wherein the historical data set is part of historical data results for performance of a previously-implemented pharmaceutical distribution mode.
277. The method of any one of embodiments 275-276, wherein the previously-implemented pharmaceutical distribution model comprises a previously-implemented distribution constraint that shares a common archetype with a distribution constraint of the plurality of distribution constraints.
278. The method of any one of embodiments 275-277, wherein the previously-implemented pharmaceutical distribution model is selected at least in part based on similarity (for example overlap of archetypes) between one or more previously-implemented distribution constraints and one or more distribution constraints of the plurality of distribution constraints.
279. The method of any one of embodiments 275-278, wherein the previously-implemented pharmaceutical distribution model is selected at least in part based on similarity (for example overlap of archetypes) between a previously-implemented performance performance metric applied to the previously-implemented pharmaceutical distribution model and the performance metric.
280. The method of any one of embodiments 272-279, wherein the data set is a synthetic data set.
281. The method of embodiment 280, wherein the data set is part of simulated computational results for performance of a synthetic pharmaceutical distribution model.
282. The method of embodiment 281, wherein the synthetic pharmaceutical distribution model comprises a synthetic distribution constraint that shares a common archetype with a distribution constraint of the plurality of distribution constraints.
283. The method of any one of the embodiments 281-282, wherein the synthetic pharmaceutical distribution model is selected at least in part based on similarity between one or more synthetic distribution constraints and one or more distribution constraints of the plurality of distribution constraints.
284. The method of embodiment 283, wherein the similarity is an overlap of archetypes.
285. The method of any one of the embodiments 281-284, wherein the synthetic pharmaceutical distribution model is selected at least in part based on similarity between a synthetic performance metric applied to the synthetic pharmaceutical distribution model and the performance metric.
286. The method of embodiment 285, wherein the similarity is an overlap of archetypes.
287. The method of any one of embodiments 272-286, wherein the adjusting comprises adding a distribution constraint to the plurality of distribution constraints.
288. The method of any one of embodiments 272-287, wherein the adjusting comprises removing a distribution constraint from the plurality of distribution constraints.
289. The method of any one of embodiments 272-287, wherein the adjusting comprises replacing a distribution constraint in the plurality of distribution constraints.
290. A computer-implemented method for distributing a pharmaceutical therapy to at least one eligible patient, comprising:
a. generating a mathematical program that comprises a first objective function and multiple distribution constraints;
b. solving the mathematical program to generate a computer-executable pharmaceutical distribution model that at least partially optimizes the first objective function;
c. performing a dynamic simulation of the computer-executable pharmaceutical distribution model based on a set of simulation assumptions to obtain simulation results;
d. calculating a value of a second objective function for the simulation results; and
e. making the computer-executable pharmaceutical distribution model, the set of simulation assumptions, the simulation results, and the second objective function accessible to a third-party computing device.
291. The method of embodiment 291, the method further comprising the features of any one of the embodiments 1-271.
292. The method of embodiment 291, the method further comprising the features of any one of the embodiments 272-289.
293. A product for distributing a pharmaceutical therapy to at least one patient, the product comprising a non-transitory computer-readable storage medium having computer-readable program code executable by a computing device to perform computer modeling and communication operations, the computer modeling and communication operations comprising:
a. configuring a computer-executable pharmaceutical distribution model that comprises a plurality of distribution constraints;
b. calculating a time-varying behavior of at least one parameter of the computer-executable pharmaceutical distribution model to obtain computational results;
c. quantifying a value of a first performance metric for the computational results;
d. adjusting the plurality of distribution constraints based at least on the computational results to form a computer-executable adjusted pharmaceutical distribution model that comprises an adjusted plurality of distribution constraints;
e. further calculating a time-varying behavior of at least one further parameter of the computer-executable adjusted pharmaceutical distribution model to obtain adjusted computational results;
f. further quantifying a value of a second performance metric for the adjusted simulation results; and
g. making the computer-executable adjusted pharmaceutical distribution model accessible to a third-party computing device.
294. The product of embodiment 293, the product further comprising the features of any one of the embodiments 1-271.
295. The product of embodiment 293, the product further comprising the features of any one of the embodiments 272-289.
296. A system for distributing a pharmaceutical therapy to at least one patient, the system comprising an analytic engine and one or more computing systems configured to perform operations comprising:
a. configuring a computer-executable pharmaceutical distribution model that comprises a plurality of distribution constraints;
b. calculating a time-varying behavior of at least one parameter of the computer-executable pharmaceutical distribution model to obtain computational results;
c. quantifying a value of a first performance metric for the computational results;
d. adjusting the plurality of distribution constraints based at least on the computational results to form a computer-executable adjusted pharmaceutical distribution model that comprises an adjusted plurality of distribution constraints;
e. further calculating a time-varying behavior of at least one further parameter of the computer-executable adjusted pharmaceutical distribution model to obtain adjusted computational results;
f. further quantifying a value of a second performance metric for the adjusted simulation results; and
g. making the computer-executable adjusted pharmaceutical distribution model accessible to a third-party computing device.
297. The system of embodiment 296, the system further performing operations of any one of the embodiments 1-271.
298. The system of embodiment 296, system further performing operations of any one of the embodiments 272-289.

In some implementations, a product, such as the product of embodiment 293, is combinable with the features of any one of the embodiments 1-271 or any one of the embodiments 272-289.

In some implementations, a system, such as the system of embodiment 296 is combinable with the features of any one of the embodiments 1-271 or any one of the embodiments 272-289.

1. A computer-implemented method for distributing a pharmaceutical therapy to at least one patient, comprising:
configuring a computer-executable pharmaceutical distribution model that comprises a plurality of distribution constraints;
calculating a time-varying behavior of at least one parameter of the computer-executable pharmaceutical distribution model to obtain computational results;
quantifying a value of a first performance metric for the computational results;
adjusting the plurality of distribution constraints based at least on the computational results to form a computer-executable adjusted pharmaceutical distribution model that comprises an adjusted plurality of distribution constraints;
further calculating a time-varying behavior of at least one further parameter of the computer-executable adjusted pharmaceutical distribution model to obtain adjusted computational results;
further quantifying a value of a second performance metric for the adjusted simulation results; and
making the computer-executable adjusted pharmaceutical distribution model accessible to a third-party computing device.

2. The method of claim 1, further comprising receiving user input that is used in the configuring.

3. The method of claim 2, further comprising providing user access to a sandbox environment for guiding one or more of the configuring, the adjusting, and the making.

4. The method of claim 3, wherein the user access is provided by one or more of a website interface, an application programming interface, and an app.

5. The method of claim 1, wherein the making comprises providing a URL for onboarding a user, the URL providing access to the adjusted pharmaceutical distribution model.

6. The method of claim 1, wherein the computational results comprises results for a plurality of time-dependent scenarios that are a function of the parameter.

7. The method of claim 6, wherein the value of the first performance metric comprises an average of results determined from the plurality of time-dependent scenarios.

8. The method of claim 6, wherein the plurality of time-dependent scenarios are generated at least in part using a random number generator.

9. The method of claim 6, wherein the computational results comprise a solution to a mathematical program that optimizes the at least one parameter so as to maximize or minimize the value of the first performance metric subject to the distribution constraints.

10. The method of claim 9, wherein mathematical program is a mixed-integer linear program.

11. The method of claim 9, wherein the mathematical program is a mixed-integer nonlinear program.

12. The method of claim 1, wherein the computational results comprise a sensitivity analysis with respect to the at least one parameter.

13. The method of claim 1, wherein the plurality of distribution constraints comprise statistical values derived from proprietary data.

14. The method of claim 13, wherein a distribution constraint of the plurality of distribution constraints comprises a statistical model derived from third-party proprietary data.

15. The method of claim 1, wherein the adjusted pharmaceutical distribution model is formed at least in part by restricting a portion of the pharmaceutical distribution model.

16. The method of claim 1, wherein the adjusting is further based at least on the value of the first performance metric.

17. The method of claim 1, wherein the making further comprises making the value of the second performance metric accessible to the third-party computing device, and the value of the second performance metric is greater than the value of the first performance metric.

18. The method of claim 1, wherein the adjusted pharmaceutical distribution model defines a restricted mode of the pharmaceutical distribution model.

19. A product for distributing a pharmaceutical therapy to at least one patient, the product comprising a non-transitory computer-readable storage medium having computer-readable program code executable by a computing device to perform computer modeling and communication operations, the computer modeling and communication operations comprising:
configuring a computer-executable pharmaceutical distribution model that comprises a plurality of distribution constraints;
calculating a time-varying behavior of at least one parameter of the computer-executable pharmaceutical distribution model to obtain computational results;
quantifying a value of a first performance metric for the computational results;
adjusting the plurality of distribution constraints based at least on the computational results to form a computer-executable adjusted pharmaceutical distribution model that comprises an adjusted plurality of distribution constraints;
further calculating a time-varying behavior of at least one further parameter of the computer-executable adjusted pharmaceutical distribution model to obtain adjusted computational results;
further quantifying a value of a second performance metric for the adjusted simulation results; and
making the computer-executable adjusted pharmaceutical distribution model accessible to a third-party computing device.

20. A system for distributing a pharmaceutical therapy to at least one patient, the system comprising an analytic engine and one or more computing systems configured to perform operations comprising:
configuring a computer-executable pharmaceutical distribution model that comprises a plurality of distribution constraints;
calculating a time-varying behavior of at least one parameter of the computer-executable pharmaceutical distribution model to obtain computational results;
quantifying a value of a first performance metric for the computational results;
adjusting the plurality of distribution constraints based at least on the computational results to form a computer-executable adjusted pharmaceutical distribution model that comprises an adjusted plurality of distribution constraints;
further calculating a time-varying behavior of at least one further parameter of the computer-executable adjusted pharmaceutical distribution model to obtain adjusted computational results;
further quantifying a value of a second performance metric for the adjusted simulation results; and
making the computer-executable adjusted pharmaceutical distribution model accessible to a third-party computing device.
